(19) United States
(12) Patent Application Publication
(10) Pub. No.: US 2025/0304905 A1
(43) Pub. Date: Oct. 2, 2025

(54) POINT OF CARE SYSTEM FOR AUTOMATICALLY PROCESSING CELLS

(71) Applicant: STEMCELL TECHNOLOGIES CANADA INC., Vancouver (CA)

(72) Inventors: Maisam DADGAR, Watertown, MA (US); Tarek ABDELJAWAD, Watertown, MA (US); Howard BERNSTEIN, Watertown, MA (US)

(21) Appl. No.: 19/097,211

(22) Filed: Apr. 1, 2025

Related U.S. Application Data
(63) Continuation of application No. 17/327,511, filed on May 21, 2021, now abandoned.
(60) Provisional application No. 63/029,230, filed on May 22, 2020.

(51) Int. Cl. C12M 1/00; C12M 1/26; C12M 1/42; C12M 3/06

(52) U.S. Cl. CPC C12M 47/04; C12M 23/16; C12M 29/00; C12M 33/14; C12M 35/04; C12M 41/14

(57) ABSTRACT

The present disclosure generally relates to a systems and methods for processing cells, and kits for use with such methods and processes. In some aspects, the system, methods, and processes can be used to produce cell therapeutics.

### CROSS-REFERENCE TO RELATED APPLICATIONS

This application is a continuation of U.S. patent application Ser. No. 17/327,511, which was filed on May 21, 2021, which claims priority to and benefit of U.S. Provisional Application No. 63/029,230 filed May 22, 2020, the entire content of each of which is hereby incorporated by reference herein in its entirety, for all purposes.

### FIELD

The present disclosure generally relates to systems and methods for processing cells, and to kits for use with such systems and methods for processing cells.

### BACKGROUND

Cell therapeutics, such as cell-based therapeutics, are designed to boost the immune response and are becoming an increasingly common treatment option for a number of diseases, such as, for example, cancer. In particular, cell therapies, such as cell-based therapies, for cancer treatment have a number of advantages over traditional treatment options, such as chemotherapy and radiation, and as such, cell therapies are becoming more prevalent in their use. These advantages can include shorter treatment times as compared to chemotherapy as in some instances only a single infusion may be needed to achieve a desired therapeutic result. An additional advantage can be a more rapid recovery time as compared to recovery from chemotherapy. A further advantage of cell therapeutics as compared to chemotherapy is that cell therapeutics can be a targeted treatment of specific molecular targets associated with cancer, as opposed to standard chemotherapies that act on all rapidly dividing normal and cancerous cells.

However, despite the benefits of currently approved cell therapeutics, such as cell-based therapeutics, the limitations in the manufacturing process for these cell therapeutics is a significant bottleneck in the field. As discussed further infra, other manufacturing processes can take on the order of days to weeks to produce the desired cell therapeutic. Moreover, these other manufacturing processes generally involve the use of a number of different instruments, each requiring its own setup and cleaning, greatly adding to the time to produce a final product. Moreover, as other methods for producing cell therapeutics require a number of different instruments and a sterile environment, a large amount of physical space that has the necessary sterile conditions, e.g., multiple clean rooms, are required to perform the manufacturing process, thereby adding significant cost to the manufacturing process.

As such, there is a need in the field for systems and methods for processing cells, such as to produce cell therapeutics, which systems and methods represent improvements over the current systems and methods.

### BRIEF SUMMARY OF THE INVENTION

The present disclosure generally relates to a system for processing cells, the system comprising: a suspension preparation subsystem comprising: a delivery media inlet; a cell isolation device configured to isolate cells; a cell suspension device configured to suspend isolated cells in delivery media thereby creating a cell suspension; a cell-deformation subsystem in fluid communication with the suspension preparation subsystem, wherein the cell-deformation subsystem comprises: a cell suspension inlet; one or more cell-deforming constrictions configured to cause perturbations in cell membranes of the cells to allow entry of a payload into the cell, thereby creating a cell suspension comprising engineered cells; a dilution subsystem in fluid communication with the cell-deformation subsystem, wherein the dilution subsystem comprises: a cell suspension inlet; a buffer inlet; a container configured to receive the cell suspension comprising engineered cells and to receive a fluid or a dry reagent that mixes with the cell suspension comprising engineered cells to create a diluted cell suspension; an incubation subsystem in fluid communication with the dilution subsystem, wherein the incubation subsystem comprises: a diluted cell suspension inlet; a container configured to receive the diluted cell suspension; and a plate configured to adjust the temperature of the diluted cell suspension in the container to create an incubated cell suspension; a cell-washing subsystem in fluid communication with the incubation subsystem, wherein the cell-washing subsystem comprises: an incubated cell suspension inlet; a preservation media inlet; a container configured to receive the incubated cell suspension and to receive preservation media that mixes with the incubated cell suspension in the container thereby suspending the cells in preservation media; and a container-filling subsystem in fluid communication with the incubation subsystem, wherein the container-filling subsystem comprises: an inlet configured to receive cells suspended in preservation media; one or more containers configured to receive cells suspended in preservation media; and one or more pumps configured to pump the cells suspended in preservation media into the one or more containers; wherein at least one of the subsystems is releasably coupled to a frame of the system.

In some aspects, the system is sterile and configured to be used in a non-sterile location. In some aspects, the system produces processed cells in about 5 hours to about 7 hours. In some aspects, the cells comprise red blood cells (RBC). In some aspects, the cells comprise RBC-derived vesicles, e.g. activating antigen carriers (AACs), tolerizing antigen carriers (TAC). In some aspects, the cells comprise peripheral blood mononuclear cells (PBMCs). In some aspects, the cells comprise activating antigen carriers (APCs). In some aspects, the cells comprise T cells, B cells, dendritic cells, monocytes, macrophages, eosinophils, basophils, natural killer (NK) cells, natural killer T (NKT) cells, mast cells or neutrophils. In some aspects, the cell suspension inlet and buffer inlet of the dilution subsystem are the same inlet. In some aspects, the buffer inlet is used to deliver fluid to the dilution subsystem. In some aspects, the buffer inlet is used to deliver dry reagent to the dilution subsystem. In some aspects, the payload comprises one or more reprogramming factors. In some aspects, the payload comprises one or more nucleic acids. In some aspects, the payload comprises one or more differentiation factors. In some aspects, the payload comprises one or more neuron reprogramming factors. In some aspects, one or more of the subsystems comprise one or more components configured to releasably couple to the system. In some aspects, the suspension preparation subsystem further comprises a wash media inlet. In some aspects, the suspension preparation subsystem further comprises a dilution media inlet. In some aspects, the suspension preparation subsystem further comprises an elutriation system configured to perform the cell isolation operation on the cells. In some aspects, the suspension preparation subsystem further comprises a leukoreduction filter system configured to perform the cell isolation operation on the cells. In some aspects, the suspension preparation subsystem further comprises a tangential flow filtration system configured to perform a buffer exchange operation. In some aspects, the suspension preparation subsystem further comprises at least one outlet configured to be coupled to at least one container for receiving cells suspended in delivery media. In some aspects, the container is a bag comprising at least one inlet and at least one outlet. In some aspects, the suspension preparation subsystem comprises a scale system configured to weigh a container. In some aspects, the scale system comprises a tension load cell. In some aspects, the scale system comprises a compression load cell. In some aspects, the scale system comprises a straight bar load cell. In some aspects, the suspension preparation subsystem further comprises an agitation system comprising a platform configured to contact a container and to rock the container up and down. In some aspects, the suspension preparation subsystem further comprises an agitation system comprising a plate configured to contact a container and to move in and out while contacting the container. In some aspects, the suspension preparation subsystem further comprises one or more pumps configured to move fluid within the subsystem or between fluidically connected subsystems. In some aspects, the suspension preparation subsystem is capable of removing plasma. In some aspects, the suspension preparation subsystem is capable of removing serum.

In some aspects, the cell-deformation subsystem further comprises a pressurization system configured to generate pressure to force the cell suspension through the one or more cell-deforming constrictions. In some aspects, the cell-deformation subsystem further comprises a preparation vessel configured to cause the cell suspension to flow through the one or more cell-deforming constrictions. In some aspects, the preparation vessel is a rigid reservoir subassembly. In some aspects, the cell-deformation subsystem further comprises a temperature control system comprising a heated plate configured to control a temperature of the cell suspension. In some aspects, the cell-deformation subsystem further comprises at least one outlet configured to be coupled to at least one container for receiving a cell suspension or a cell suspension comprising engineered cells. In some aspects, the container is a bag comprising at least one inlet and at least one outlet. In some aspects, the cell-deformation subsystem further comprises an agitation system comprising a platform in contact with the at least one container configured to rock the container up and down. In some aspects, the agitation system comprising a platform in contact with the at least one container is configured to rock the container up and down to agitate the cell suspension to promote homogeneity of the cell suspension or the cell suspension comprising engineered cells. In some aspects, the cell-deformation subsystem further comprises an agitation system comprising a plate configured to contact the container and to move in and out while contacting the container. In some aspects, wherein the dilution subsystem comprises a scale system for weighing the container. In some aspects, the scale system for weighing the container comprises a tension load cell. In some aspects, the scale system for weighing the container comprises a compression load cell. In some aspects, the scale system for weighing the container comprises a straight bar load cell. In some aspects, the dilution subsystem comprises a scale system for measuring an amount of buffer added to the cells of the cell suspension comprising engineered cells. In some aspects, the scale system for measuring the amount of buffer comprises a tension load cell. In some aspects, the scale system for measuring the amount of buffer comprises a compression load cell. In some aspects, the scale system for measuring the amount of buffer comprises a straight bar load cell. In some aspects, the dilution subsystem comprises at least one outlet configured to be coupled to the container configured to receive the cell suspension comprising engineered cells. In some aspects, the container is a bag comprising at least one inlet and at least one outlet. In some aspects, the dilution subsystem further comprises an agitation system comprising a platform in contact with the container configured to rock the container up and down. In some aspects, the first agitation system comprising a platform in contact with the container is configured to rock the container up and down to agitate the cell suspension to promote homogeneity of the cell suspension comprising engineered cells or the diluted cell suspension. In some aspects, the dilution subsystem further comprises a first agitation system comprising a plate configured to contact a container and to move in and out while contacting the container. In some aspects, the dilution subsystem further comprises an illumination system configured to illuminate the cell suspension comprising engineered cells and the diluted cell suspension. In some aspects, the dilution subsystem further comprises a second agitation system comprising a platform in contact with the container configured to rock the container up and down. In some aspects, the second agitation system is configured to rock the container up and down to agitate the cell suspension comprising engineered cells or the diluted cell suspension to promote homogeneity of the cell suspension comprising engineered cells or the diluted cell suspension. In some aspects, the dilution subsystem further comprises a second agitation system comprising a plate configured to contact the container and to move in and out while contacting the container. In some aspects, the dilution subsystem comprises at least one pump configured to move fluid between fluidically connected subsystems.

In some aspects, the incubation subsystem comprises a temperature control device configured to adjust a temperature of the diluted cell suspension. In some aspects, the temperature control device is a heated plate. In some aspects, the incubation subsystem comprises a scale system configured to measure an amount of the buffer added to the cells of the diluted cell suspension. In some aspects, the scale system comprises a tension load cell. In some aspects, the scale system comprises a compression load cell. In some aspects, the scale system comprises a straight bar load cell. In some aspects, the incubation subsystem comprises a first agitation system comprising a platform in contact with the container configured to rock the container up and down. In some aspects, the first agitation system comprising a platform in contact with the container is configured to rock the container up and down to agitate the diluted cell suspension to promote homogeneity of the diluted cell suspension or the incubated cell suspension. In some aspects, the incubation subsystem comprises a second agitation system comprising a platform in contact with the container configured to rock the container up and down. In some aspects, the first agitation system comprising a platform in contact with the container is configured to rock the container up and down to agitate the cell suspension to promote homogeneity of the diluted cell suspension or the incubated cell suspension. In some aspects, the incubation subsystem comprises at least one pump configured to move fluid between fluidically connected subsystems. In some aspects, the cell-washing subsystem comprises a tangential flow filtration system configured to perform a buffer exchange operation. In some aspects, the cell-washing subsystem comprises a scale system configured to measure an amount of the buffer added to the cells during the buffer exchange operation. In some aspects, the scale system is a tension load cell. In some aspects, the scale system is a compression load cell. In some aspects, the scale system is a straight bar load cell. In some aspects, the cell-washing subsystem comprises at least one outlet configured to be coupled to the container. In some aspects, the container is a bag comprising at least one inlet and at least one outlet. In some aspects, the cell-washing subsystem comprises an agitation system comprising a platform in contact with the container configured to rock the container up and down. In some aspects, the agitation system comprising a platform in contact with the container is configured to rock the container up and down to agitate the incubated cell suspension or the cells suspended in preservation media to promote homogeneity of the diluted cell suspension or the cells suspended in preservation media. In some aspects, the cell-washing subsystem comprises an illumination system configured to illuminate the diluted cell suspension or the cells suspended in the preservation media. In some aspects, the preservation media is a cryoprotectant media. In some aspects, the container-filling subsystem comprises a scale system configured to measure an amount of the cells suspended in the preservation media added to the one or more containers. In some aspects, the scale system comprises a tension load cell. In some aspects, the scale system comprises a compression load cell. In some aspects, the scale system comprises a straight bar load cell. In some aspects, the container-filling subsystem comprises an agitation system comprising a platform in contact with the container configured to rock the container up and down. In some aspects, the agitation system comprising a platform in contact with the container is configured to rock the container up and down to agitate the cells suspended in preservation media to promote homogeneity of the cells suspended in the preservation media in the one or more containers. In some aspects, the container-filling subsystem comprises an illumination system configured to illuminate the cells suspended in the preservation media in the one or more containers. In some aspects, the container-filling subsystem comprises one or more outlets configured to be coupled to the one or more containers. In some aspects, the one or more containers of the cell-washing subsystem comprises one or more bags comprising at least one inlet and at least one outlet. In some aspects, the container-filling subsystem comprises at least one pump configured to move fluid within the subsystem or between fluidically connected subsystems. In some aspects, the system comprises one or more pumps configured to pump fluid between two or more of the subsystems. In some aspects, the system is used in a non-sterile environment. In some aspects, the system is an automatic system for processing cells. In some aspects, the system processes cells for cell therapeutics, such as cell-based therapeutics. In some aspects, the suspension preparation subsystem further comprises one or more of: a cell aggregate filter, a leukoreduction filter, tubing, a tube fitting, a connector, a clamp, a sampling bulb, a carboy, and an air filter. In some aspects, the cell-deformation subsystem further comprises one or more of: a rigid sample vessel, a cell-aggregate filter, a rigid preparation vessel, one or more microfluidic chip cartridges, one or more microfluidic chips, tubing, a tube fitting, a connector, a clamp, an air filter, and a barrel filter. In some aspects, the dilution subsystem further comprises one or more of: a cell aggregate filter, tubing, a tube fitting, a connector, a clamp, a sampling bulb, and a carboy. In some aspects, the incubation subsystem further comprises one or more of: a cell aggregate filter, tubing, a tube fitting, a connector, and a clamp. In some aspects, the cell-washing subsystem further comprises one or more of: a cell aggregate filter, tubing, a tube fitting, a connector, a clamp, a sampling bulb, an air filter, and a carboy. In some aspects, the container-filling subsystem further comprises one or more of: a cell aggregate filter, tubing, a tube fitting, a connector, a clamp, and a sampling bulb.

Moreover, the present disclosure generally relates to a method for processing cells, wherein the method is performed by a system comprising one or more of a suspension preparation subsystem, a cell-deformation subsystem, a dilution subsystem, an incubation subsystem, a cell-washing subsystem, and a container-filling subsystem, the method comprising: i. at the suspension preparation subsystem: (1) receiving cells from a container; (2) performing a cell isolation operation on the cells thereby producing isolated cells; (3) receiving a delivery media via a delivery media inlet; and (4) producing a cell suspension by suspending the isolated cells to in the delivery media, thereby producing a cell suspension; ii. at the cell-deformation subsystem: (1) receiving flow of the cell suspension from the suspension preparation subsystem; (2) flowing the cell suspension through one or more cell-deforming constrictions configured to cause perturbations in cell membranes of the cells to allow entry of a payload into the cell, thereby producing a cell suspension comprising engineered cells; iii. at the dilution subsystem: (1) receiving flow of the cell suspension comprising engineered cells from the cell-deformation subsystem; (2) receiving a fluid or dry reagent via a buffer inlet; and (3) producing a diluted cell suspension by mixing the cell suspension comprising engineered cells with the fluid or dry reagent; iv. at the incubation subsystem: (1) receiving flow of the diluted cell suspension from the dilution subsystem; and (2) adjusting a temperature of the diluted cell suspension, thereby producing an incubated cell suspension; v. at the cell-washing subsystem: (1) receiving flow of the incubated cell suspension from the incubation subsystem; (2) performing a buffer exchange operation on the cells to suspend the cells in a preservation media; and vi. at the container filling subsystem: (1) receiving flow of the cells suspended in preservation media from the cell-washing subsystem; and (2) introducing the cells suspended in preservation media into one or more containers.

In some aspects, the cells comprise red blood cells (RBC). In some aspects, the cells comprise RBC-derived vesicles, e.g. activating antigen carriers (AACs), tolerizing antigen carriers (TAC). In some aspects, the cells comprise peripheral blood mononuclear cells (PBMCs). In some aspects, the cells comprise activating antigen carriers (APCs). In some aspects, the cells comprise T cells, B cells, dendritic cells, monocytes, macrophages, eosinophils, basophils, natural killer (NK) cells, natural killer T (NKT) cells, mast cells or neutrophils. In some aspects, the method removes plasma. In some aspects, the method removes serum. In some aspects, the method is performed in about 5 to about 7 hours. In some aspects, the method produces cell therapeutics, e.g., cell-based therapeutics. In some aspects, the payload comprises one or more reprogramming factors. In some aspects, the payload comprises one or more nucleic acids. In some aspects, the payload comprises one or more differentiation factors. In some aspects, the payload comprises one or more neuron reprogramming factors. In some aspects, the method is an automatic method for processing cells. In some aspects, the method is performed by a sterile system in a non-sterile environment.

Moreover, the present disclosure generally relates to a kit for use in a system for processing cells, the kit comprising one or more of: i. a first kit comprising releasably couplable components configured to be releasably couplable to a frame of a suspension preparation subsystem of the system, wherein the first set of releasably couplable components comprises: (1) a cell isolation device; and/or (2) a cell suspension device; ii. a second kit comprising releasably couplable components configured to be releasably couplable to a frame of a cell-deformation subsystem of the system, wherein the second set of releasably couplable components comprises one or more microfluidic chips comprising one or more cell-deforming constrictions through which cells may be forced to cause perturbation of membranes of the cells; iii. a third kit comprising releasably couplable components configured to be releasably couplable to a frame of a dilution subsystem of the system, wherein the third set of releasably couplable components comprises at least one cell aggregate filter; iv. a fourth kit comprising releasably couplable components configured to be releasably couplable to a frame of an incubation subsystem of the system, wherein the fourth set of releasably couplable components comprises at least one cell aggregate filter; v. a fifth kit comprising releasably couplable components configured to be releasably couplable to a frame of a cell-washing subsystem of the system, wherein the fifth set of releasably couplable components comprises a second tangential flow filtration membrane assembly; and vi. a sixth kit comprising releasably couplable components configured to be releasably couplable to a frame of a container-filling subsystem of the system, wherein the sixth set of releasably couplable components comprises at least one cell aggregate filter.

In some aspects, one or more of the components of one or more of the kits are configured to be fluidly connected to one or more components of the corresponding subsystem of the system. In some aspects, the cell isolation device comprises at least one elutriation device. In some aspects, the cell isolation device comprises at least one leukoreduction filter. In some aspects, the first kit comprises a cell isolation device. In some aspects, the cell suspension device is a tangential flow filtration membrane assembly. In some aspects, i. one or more of the releasably couplable components of the second kit of releasably couplable components is configured to be fluidly connected to one or more of the releasably couplable components of the first kit of releasably couplable components; ii. one or more of the releasably couplable components of the third kit of releasably couplable components is configured to be fluidly connected to one or more of the releasably couplable components of the second set of releasably couplable components; iii. one or more of the releasably couplable components of the fourth kit of releasably couplable components is configured to be fluidly connected to one or more of the releasably couplable components of the third kit of releasably couplable components; iv. one or more of the releasably couplable components of the fifth kit of releasably couplable components is configured to be fluidly connected to one or more of the releasably couplable components of the fourth kit of releasably couplable components; and/or v. one or more of the releasably couplable components of the sixth kit of releasably couplable components is configured to be fluidly connected to one or more of the releasably couplable components of the fifth kit of releasably couplable components. In some aspects, the first kit of releasably couplable components comprises a cell aggregate filter. In some aspects, the first kit of releasably couplable components comprises a leukoreduction filter. In some aspects, the first kit of releasably couplable components comprises a container. In some aspects, the first kit of releasably couplable components comprises a tangential flow filtration filter assembly. In some aspects, the first kit of releasably couplable components comprises one or more of: tubing, a tube fitting, a connector, a clamp, a sampling bulb, a carboy, an air filter, and a tangential flow filtration filter assembly. In some aspects, the second kit of releasably couplable components comprises a rigid sample vessel. In some aspects, the second kit of releasably couplable components comprises a cell-aggregate filter. In some aspects, the second kit of releasably couplable components comprises a preparation vessel. In some aspects, the second kit of releasably couplable components comprises one or more microfluidic chips. In some aspects, the second kit of releasably couplable components comprises one or more microfluidic chip cartridges. In some aspects, the second kit of releasably couplable components comprises one or more of: tubing, a tube fitting, a connector, a clamp, a container, a bag, an air filter, and a barrel filter. In some aspects, the third kit of releasably couplable components comprises a container. In some aspects, the third kit of releasably couplable components comprises a cell aggregate filter. In some aspects, the third kit of releasably couplable components comprises one or more of: tubing, a tube fitting, a connector, a clamp, a sampling bulb, and a carboy. In some aspects, the fourth kit of releasably couplable components comprises a cell aggregate filter. In some aspects, the fourth kit of releasably couplable components comprises one or more containers. In some aspects, the fourth kit of releasably couplable components comprises one or more of: tubing, a tube fitting, a connector, and a clamp. In some aspects, the fifth kit of releasably couplable components comprises a cell aggregate filter. In some aspects, the fifth kit of releasably couplable components comprises a container. In some aspects, the fifth kit of releasably couplable components comprises a tangential flow filtration filter assembly. In some aspects, the fifth kit of releasably couplable components comprises one or more of: tubing, a tube fitting, a connector, a clamp, a sampling bulb, an air filter, and a carboy. In some aspects, the sixth kit of releasably couplable components comprises a container. In some aspects, the container is a cryopreservation bag. In some aspects, the sixth kit of releasably couplable components comprises a cell aggregate filter. In some aspects, the sixth kit of releasably couplable components comprises one or more of: tubing, a tube fitting, a connector, a clamp, and a sampling bulb. In some aspects, the kit comprises the first, second, third, fourth, fifth, and sixth kits. In some aspects, the kit is packaged in an accordion tray package. In some aspects, the kit is packaged as a rollable sheet. In some aspects, each kit is packaged separately. In some aspects, at least two kits are packaged together. I some aspects, the kit is sterile.

Moreover, the present disclosure generally relates to a system for processing cells, the system comprising: a suspension preparation subsystem comprising: a cell isolation device; a tangential flow filtration membrane assembly; a cell aggregate filter; a leukoreduction filter; and one or more containers; a cell-deformation subsystem in fluid communication with the suspension preparation subsystem, wherein the cell-deformation subsystem comprises: one or more microfluidic chips; a rigid sample vessel, a cell-aggregate filter; a dilution subsystem in fluid communication with the cell-deformation subsystem, wherein the dilution subsystem comprises: a container, a cell aggregate filter; an incubation subsystem in fluid communication with the dilution subsystem, wherein the incubation subsystem comprises: a cell aggregate filter; a container; a cell-washing subsystem in fluid communication with the incubation subsystem, wherein the cell-washing subsystem comprises: a tangential flow filtration membrane assembly; a cell aggregate filter; a container; and a container-filling subsystem in fluid communication with the incubation subsystem, wherein the container-filling subsystem comprises: a container, a cell aggregate filter; wherein subsystems i.-vi. are releasably coupled to a frame of the system.

Furthermore, the present disclosure generally relates to a system for processing cells, the system comprising: a suspension preparation subsystem comprising: a delivery media inlet; a cell isolation device configured to isolate cells; a cell suspension device configured to suspend isolated cells in delivery media thereby creating a cell suspension; a payload entry subsystem in fluid communication with the suspension preparation subsystem, wherein the payload entry subsystem comprises: a cell suspension inlet; a payload entry component to cause perturbations in cell membranes of the cells to allow entry of a payload into the cell; a dilution subsystem in fluid communication with the cell-deformation subsystem, wherein the dilution subsystem comprises: a cell suspension inlet; a buffer inlet; a container configured to receive the cell suspension comprising engineered cells and to receive buffer that mixes with the cell suspension comprising engineered cells to create a diluted cell suspension; an incubation subsystem in fluid communication with the dilution subsystem, wherein the incubation subsystem comprises: a diluted cell suspension inlet; a container configured to receive the diluted cell suspension; and a plate configured to adjust the temperature of the diluted cell suspension in the container to create an incubated cell suspension; a cell-washing subsystem in fluid communication with the incubation subsystem, wherein the cell-washing subsystem comprises: an incubated cell suspension inlet; a preservation media inlet; a container configured to receive the incubated cell suspension and to receive preservation media that mixes with the incubated cell suspension in the container thereby suspending the cells in preservation media; a container-filling subsystem in fluid communication with the incubation subsystem, wherein the container-filling subsystem comprises: an inlet configured to receive cells suspended in preservation media; one or more containers configured to receive cells suspended in preservation media; and one or more pumps configured to pump the cells suspended in preservation media into the one or more containers; wherein subsystems i.-vi. are releasably couplable to a frame. In some aspects, the payload entry component comprises an electroporation device. In some aspects, the payload entry component comprises one or more cell-deforming constrictions through which the cell suspension flows, which cell-deforming constrictions are configured to cause perturbations in cell membranes of the cells to allow entry of a payload into the cell.

Moreover, the present disclosure generally relates to a system for processing cells, the system comprising: a suspension preparation subsystem; a cell-deformation subsystem in fluid communication with the suspension preparation subsystem; a dilution subsystem in fluid communication with the cell-deformation subsystem; an incubation subsystem in fluid communication with the dilution subsystem; a cell-washing subsystem in fluid communication with the incubation subsystem; a container-filling subsystem in fluid communication with the incubation subsystem; wherein at least one of the subsystems is releasably coupled to a frame of the system.

Furthermore, the present disclosure generally relates to a system for processing cells, the system comprising: a suspension preparation subsystem; a cell-deformation subsystem in fluid communication with the suspension preparation subsystem; a temperature control subsystem in fluid communication with the cell-deformation subsystem; a cell-washing subsystem in fluid communication with the temperature control subsystem; a container-filling subsystem in fluid communication with the incubation subsystem; wherein at least one of the subsystems is releasably coupled to a frame of the system.

Moreover, the present disclosure generally relates to a system for processing cells, the system comprising: a suspension preparation subsystem comprising: a delivery media inlet; a cell suspension device configured to suspend cells in delivery media thereby creating a cell suspension; a cell-deformation subsystem in fluid communication with the suspension preparation subsystem, wherein the cell-deformation subsystem comprises: a cell suspension inlet; one or more cell-deforming constrictions configured to cause perturbations in cell membranes of the cells to allow entry of a payload into the cell, thereby creating a cell suspension comprising engineered cells; a dilution subsystem in fluid communication with the cell-deformation subsystem, wherein the dilution subsystem comprises: a cell suspension inlet; a buffer inlet; a container configured to receive the cell suspension comprising engineered cells and to receive a fluid or a dry reagent that mixes with the cell suspension comprising engineered cells to create a diluted cell suspension; an incubation subsystem in fluid communication with the dilution subsystem, wherein the incubation subsystem comprises: a diluted cell suspension inlet; a container configured to receive the diluted cell suspension; and a plate configured to adjust the temperature of the diluted cell suspension in the container to create an incubated cell suspension; a cell-washing subsystem in fluid communication with the incubation subsystem, wherein the cell-washing subsystem comprises: an incubated cell suspension inlet; a preservation media inlet; a container configured to receive the incubated cell suspension and to receive preservation media that mixes with the incubated cell suspension in the container thereby suspending the cells in preservation media; and a container-filling subsystem in fluid communication with the incubation subsystem, wherein the container-filling subsystem comprises: an inlet configured to receive cells suspended in preservation media; one or more containers configured to receive cells suspended in preservation media; and one or more pumps configured to pump the cells suspended in preservation media into the one or more containers; wherein at least one of the subsystems is releasably coupled to a frame of the system.

Furthermore, the present disclosure generally relates to a system for processing cells, the system comprising: a suspension preparation subsystem comprising: a delivery media inlet; a cell isolation device configured to isolate cells; a cell suspension device configured to suspend isolated cells in delivery media thereby creating a cell suspension; a cell-deformation subsystem in fluid communication with the suspension preparation subsystem, wherein the cell-deformation subsystem comprises: a cell suspension inlet; one or more cell-deforming constrictions configured to cause perturbations in cell membranes of the cells to allow entry of a payload into the cell, thereby creating a cell suspension comprising engineered cells; a temperature control subsystem in fluid communication with the cell-deformation subsystem, wherein the temperature control subsystem comprises: a cell suspension inlet; a buffer inlet; a container configured to receive the cell suspension comprising engineered cells and to receive a fluid or a dry reagent that mixes with the cell suspension comprising engineered cells to create a diluted cell suspension; a plate configured to adjust the temperature of the diluted cell suspension in the container to create an incubated cell suspension; a cell-washing subsystem in fluid communication with the temperature control subsystem, wherein the cell-washing subsystem comprises: an incubated cell suspension inlet; a preservation media inlet; a container configured to receive the incubated cell suspension and to receive preservation media that mixes with the incubated cell suspension in the container thereby suspending the cells in preservation media; and a container-filling subsystem in fluid communication with the incubation subsystem, wherein the container-filling subsystem comprises: an inlet configured to receive cells suspended in preservation media; one or more containers configured to receive cells suspended in preservation media; and one or more pumps configured to pump the cells suspended in preservation media into the one or more containers; wherein at least one of the subsystems is releasably coupled to a frame of the system. In some aspects, the system is an automatic system for processing cells.

Moreover, the present disclosure generally relates to a system for automatically processing cells, the system comprising: a suspension preparation subsystem comprising: a delivery media inlet; a cell isolation device configured to isolate cells; a cell suspension device configured to suspend isolated cells in delivery media thereby creating a cell suspension; a cell-deformation subsystem in fluid communication with the suspension preparation subsystem, wherein the cell-deformation subsystem comprises: a cell suspension inlet; one or more cell-deforming constrictions configured to cause perturbations in cell membranes of the cells to allow entry of a payload into the cell, thereby creating a cell suspension comprising engineered cells; a dilution subsystem in fluid communication with the cell-deformation subsystem, wherein the dilution subsystem comprises: a cell suspension inlet; a buffer inlet; a container configured to receive the cell suspension comprising engineered cells and to receive a fluid or a dry reagent that mixes with the cell suspension comprising engineered cells to create a diluted cell suspension; an incubation subsystem in fluid communication with the dilution subsystem, wherein the incubation subsystem comprises: a diluted cell suspension inlet; a container configured to receive the diluted cell suspension; and a plate configured to adjust the temperature of the diluted cell suspension in the container to create an incubated cell suspension; a cell-washing subsystem in fluid communication with the incubation subsystem, wherein the cell-washing subsystem comprises: an incubated cell suspension inlet; a preservation media inlet; a container configured to receive the incubated cell suspension and to receive preservation media that mixes with the incubated cell suspension in the container thereby suspending the cells in preservation media; and a container-filling subsystem in fluid communication with the incubation subsystem, wherein the container-filling subsystem comprises: an inlet configured to receive cells suspended in preservation media; one or more containers configured to receive cells suspended in preservation media; and one or more pumps configured to pump the cells suspended in preservation media into the one or more containers; wherein at least one of the subsystems is releasably coupled to a frame of the system, and further wherein the system is sterile and configured to be used in a non-sterile location.

Furthermore, the present disclosure generally relates to a method for processing cells, wherein the method is performed by a system comprising one or more of a suspension preparation subsystem, a cell-deformation subsystem, a dilution subsystem, an incubation subsystem, a cell-washing subsystem, and a container-filling subsystem, the method comprising: i. at the suspension preparation subsystem: (1) receiving cells; (2) performing a cell isolation operation on the cells thereby producing isolated cells; (3) receiving a delivery media; and (4) producing a cell suspension by suspending the isolated cells to in the delivery media, thereby producing a cell suspension; ii. at the cell-deformation subsystem: (1) receiving flow of the cell suspension from the suspension preparation subsystem; (2) flowing the cell suspension through one or more cell-deforming constrictions configured to cause perturbations in cell membranes of the cells to allow entry of a payload into the cell, thereby producing a cell suspension comprising engineered cells; iii. at the dilution subsystem: (1) receiving flow of the cell suspension comprising engineered cells from the cell-deformation subsystem; (2) receiving a fluid or dry reagent; and (3) producing a diluted cell suspension by mixing the cell suspension with the fluid or dry reagent; iv. at the incubation subsystem: (1) receiving flow of the diluted cell suspension from the dilution subsystem; and (2) adjusting a temperature of the diluted cell suspension, thereby producing an incubated cell suspension; v. at the cell-washing subsystem: (1) receiving flow of the incubated cell suspension from the incubation subsystem; (2) performing a buffer exchange operation on the cells to suspend the cells in a preservation media; and vi. at the container filling subsystem: (1) receiving flow of the cells suspended in preservation media from the cell-washing subsystem; and (2) introducing the cells suspended in preservation media into one or more containers.

Furthermore, the present disclosure generally relates to a method for processing cells, wherein the method is performed by a system comprising one or more of a suspension preparation subsystem, a cell-deformation subsystem, a temperature control subsystem, the method comprising: i. at the suspension preparation subsystem: (1) receiving cells from a container; (2) performing a cell isolation operation on the cells thereby producing isolated cells; (3) receiving a delivery media via a delivery media inlet; and (4) producing a cell suspension by suspending the isolated cells to in the delivery media, thereby producing a cell suspension; ii. at the cell-deformation subsystem: (1) receiving flow of the cell suspension from the suspension preparation subsystem; (2) flowing the cell suspension through one or more cell-deforming constrictions configured to cause perturbations in cell membranes of the cells to allow entry of a payload into the cell, thereby producing a cell suspension comprising engineered cells; iii. at the temperature control subsystem: (1) receiving flow of the cell suspension comprising engineered cells from the cell-deformation subsystem; (2) receiving a fluid or dry reagent via an inlet; (3) producing a diluted cell suspension by mixing the cell suspension with the fluid or dry reagent; and (4) adjusting a temperature of the diluted cell suspension, thereby producing an incubated cell suspension; iv. at the cell-washing subsystem: (1) receiving flow of the incubated cell suspension from the temperature control subsystem; (2) performing a buffer exchange operation on the cells to suspend the cells in a preservation media; and v. at the container filling subsystem: (1) receiving flow of the cells suspended in preservation media from the cell-washing subsystem; and (2) introducing the cells suspended in preservation media into one or more containers. In some aspects, the method is an automatic method for processing cells. In some aspects, the method is performed by a sterile system in a non-sterile environment.

Moreover, the present disclosure generally relates to a method for automatically processing cells, wherein the method is performed by a sterile system in a nonsterile environment, and further wherein the system comprises one or more of a suspension preparation subsystem, a cell-deformation subsystem, a dilution subsystem, an incubation subsystem, a cell-washing subsystem, and a container-filling subsystem, the method comprising: i. at the suspension preparation subsystem: (1) receiving cells from a container; (2) performing a cell isolation operation on the cells thereby producing isolated cells; (3) receiving a delivery media via a delivery media inlet; and (4) producing a cell suspension by suspending the isolated cells to in the delivery media, thereby producing a cell suspension; ii. at the cell-deformation subsystem: (1) receiving flow of the cell suspension from the suspension preparation subsystem; (2) flowing the cell suspension through one or more cell-deforming constrictions configured to cause perturbations in cell membranes of the cells to allow entry of a payload into the cell, thereby producing a cell suspension comprising engineered cells; iii. at the dilution subsystem: (1) receiving flow of the cell suspension comprising engineered cells from the cell-deformation subsystem; (2) receiving a fluid or dry reagent via a buffer inlet; and (3) producing a diluted cell suspension by mixing the cell suspension comprising engineered cells with the fluid or dry reagent; iv. at the incubation subsystem: (1) receiving flow of the diluted cell suspension from the dilution subsystem; and (2) adjusting a temperature of the diluted cell suspension, thereby producing an incubated cell suspension; v. at the cell-washing subsystem: (1) receiving flow of the incubated cell suspension from the incubation subsystem; (2) performing a buffer exchange operation on the cells to suspend the cells in a preservation media; and vi. at the container filling subsystem: (1) receiving flow of the cells suspended in preservation media from the cell-washing subsystem; and (2) introducing the cells suspended in preservation media into one or more containers.

Furthermore, the present disclosure generally relates to a method for processing cells, wherein the method is performed by a system comprising one or more of a suspension preparation subsystem, a cell-deformation subsystem, a dilution subsystem, an incubation subsystem, a cell-washing subsystem, and a container-filling subsystem, the method comprising: i. at the suspension preparation subsystem: (1) receiving cells from a container; (2) receiving a delivery media via a delivery media inlet; and (3) producing a cell suspension by suspending the cells to in the delivery media, thereby producing a cell suspension; ii. at the cell-deformation subsystem: (1) receiving flow of the cell suspension from the suspension preparation subsystem; (2) flowing the cell suspension through one or more cell-deforming constrictions configured to cause perturbations in cell membranes of the cells to allow entry of a payload into the cell, thereby producing a cell suspension comprising engineered cells; iii. at the dilution subsystem: (1) receiving flow of the cell suspension comprising engineered cells from the cell-deformation subsystem; (2) receiving a fluid or dry reagent via a buffer inlet; and (3) producing a diluted cell suspension by mixing the cell suspension comprising engineered cells with the fluid or dry reagent; iv. at the incubation subsystem: (1) receiving flow of the diluted cell suspension from the dilution subsystem; and (2) adjusting a temperature of the diluted cell suspension, thereby producing an incubated cell suspension; v. at the cell-washing subsystem: (1) receiving flow of the incubated cell suspension from the incubation subsystem; (2) performing a buffer exchange operation on the cells to suspend the cells in a preservation media; and vi. at the container filling subsystem: (1) receiving flow of the cells suspended in preservation media from the cell-washing subsystem; and (2) introducing the cells suspended in preservation media into one or more containers.

Moreover, the present disclosure generally relates to a kit for use in a system for processing cells, the kit comprising one or more of: i. a first kit comprising releasably couplable components configured to be releasably couplable to a frame of a suspension preparation subsystem of the system, wherein the first set of releasably couplable components comprises: (1) a cell isolation device; and/or (2) a cell suspension device; ii. a second kit comprising releasably couplable components configured to be releasably couplable to a frame of a cell-deformation subsystem of the system, wherein the second set of releasably couplable components comprises one or more microfluidic chips comprising one or more cell-deforming constrictions through which cells may be forced to cause perturbation of membranes of the cells; iii. a third kit comprising releasably couplable components configured to be releasably couplable to a frame of a temperature control subsystem of the system, wherein the third set of releasably couplable components comprises at least one cell aggregate filter; iv. a fourth kit comprising releasably couplable components configured to be releasably couplable to a frame of a cell-washing subsystem of the system, wherein the fifth set of releasably couplable components comprises a second tangential flow filtration membrane assembly; and v. a fifth kit comprising releasably couplable components configured to be releasably couplable to a frame of a container-filling subsystem of the system, wherein the sixth set of releasably couplable components comprises at least one cell aggregate filter.

### BRIEF DESCRIPTION OF THE DRAWINGS

FIG. 1 presents flow charts comparing a peripheral blood mononuclear cell (“PBMC”) manufacturing process that does not comprise use of a point of care system (Current PBMC Mfg. Process) and a PBMC manufacturing process that comprises use of a point of care system (POC Mfg. Process) in an embodiment.

FIG. 2 presents a schematic representation of PBMC processing that does not comprise use of a point of care system for processing cells in an embodiment.

FIG. 3 presents a schematic representation of PBMC manufacturing that does not comprise use of a point of care system in an embodiment.

FIG. 4 presents a table comparing unit operation times for a method of cell processing that does not comprise use of a point of care system compared to a method of cell processing comprising use of a point of care system in an embodiment.

FIG. 5A presents a schematic representation of the reservoirs used by a cell processing system that does not comprise a point of care system in an embodiment.

FIG. 5B presents a schematic representation of the reservoirs of a point of care cell processing system in an embodiment.

FIG. 6 presents schematic representations of two views of a point of care system in an embodiment.

FIG. 7 presents a schematic representation of a point of care system in an embodiment.

FIG. 8 presents a schematic representation of a point of care system in an embodiment.

FIG. 9 presents a schematic representation of a disposable kit for use with a point of care system in an embodiment.

FIG. 10 presents a schematic representation of a point of care system in an embodiment.

FIG. 11 presents a schematic representation of a disposable waste tank kit in an embodiment.

FIG. 12 presents a schematic representation of a suspension preparation subsystem (Zone 1) disposable kit for cell processing in an embodiment.

FIG. 13 presents a schematic representation of a suspension preparation subsystem (Zone 1) disposable kit for cell processing in an embodiment.

FIG. 14 presents a schematic representation of a cell-deformation subsystem (Zone 2) disposable kit in an embodiment.

FIG. 15 presents a schematic representation of a dilution subsystem (Zone 3) disposable kit in an embodiment.

FIG. 16 presents a schematic representation of an incubation subsystem (Zone 4) disposable kit in an embodiment.

FIG. 17 presents a schematic representation of a cell-washing subsystem (Zone 5) disposable kit in an embodiment.

FIG. 18 presents a schematic representation of a container-filling subsystem (Zone 6) disposable kit in an embodiment.

FIG. 19 presents an image of a heated plate for use with a point of care system in an embodiment.

FIG. 20 presents a schematic representation of a bag in an embodiment.

FIG. 21 presents an image of a bag filling and weigh station for use with a point of care system in an embodiment.

FIG. 22 presents a schematic representation of an accordion tray installation of disposable kits for a point of care system in an embodiment.

FIG. 23 presents a schematic representation of a shower curtain installation of disposable kits for a point of care system in an embodiment.

FIG. 24A present a schematic representation of a direct thermoelectric cooler (TEC) liquid cooling system for use with a point of care system in an embodiment.

FIG. 24B present a schematic representation of a direct TEC liquid cooling system for use with a point of care system in an embodiment.

FIG. 25 presents a schematic representation of an agitation system for use with a point of care system in an embodiment.

FIG. 26 presents a schematic representation of a point of care system PBMC process flow in an embodiment.

FIG. 27 presents schematic representations of a suspension preparation subsystem (Zone 1) process flow for PBMC in an embodiment.

FIG. 28 presents schematic representations of a suspension preparation subsystem (Zone 1) process flow for RBC in an embodiment.

FIG. 29 presents schematic representations of cell-deformation (Zone 2) and dilution subsystem (Zone 3) process flows for PBMC in an embodiment.

FIG. 30 presents schematic representations of cell-deformation (Zone 2) and dilution subsystem (Zone 3) process flows for RBC in an embodiment.

FIG. 31 presents schematic representations of incubation subsystem (Zone 4), cell-washing subsystem (Zone 5), and container-filling subsystem (Zone 6) process flows for PBMC and RBC in an embodiment.

FIG. 32 presents a schematic representation of a pressure integrity test for use with a point of care system in an embodiment.

FIG. 33 presents a schematic representation of buffer priming sectors of a point of care system in an embodiment.

FIG. 34 presents a schematic representation of a cell deformation process in an embodiment.

FIG. 35 presents a schematic representation of a point of care system RBC process flow in an embodiment.

### DETAILED DESCRIPTION OF THE INVENTION

### 1. Systems for Processing Cells

a. Background

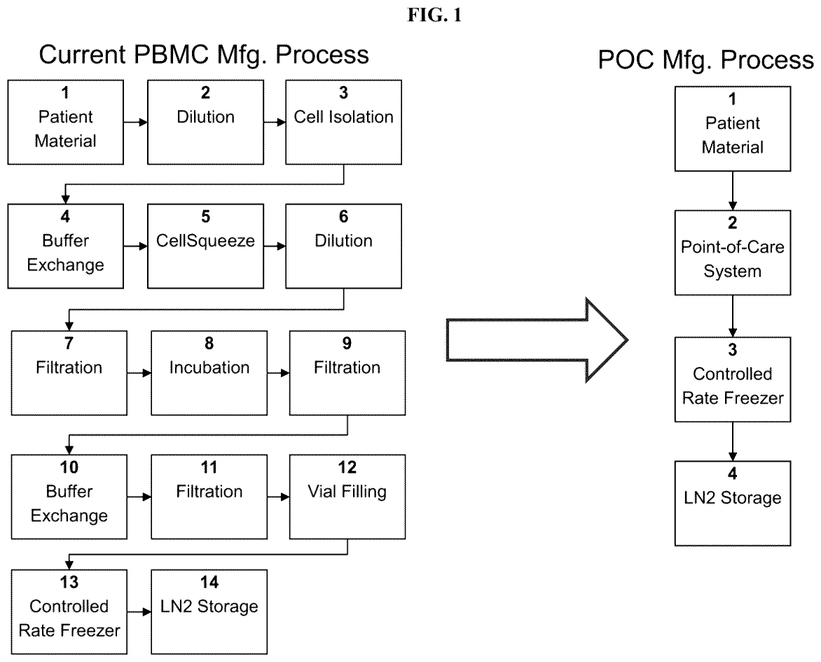

As discussed supra, other systems and methods for cell processing cells can take on the order of days to weeks to produce the desired cell therapeutic. Moreover, other manufacturing processes generally involve the use of a number of different instruments, each requiring its own setup and cleaning, greatly adding to the time to produce a final product. Furthermore, as other methods for producing cell therapies require a number of different instruments and a sterile environment, a large amount of physical space, i.e., multiple clean rooms, are required to perform the manufacturing process. For instance, FIG. 1 presents a flow diagram of a cell processing manufacturing process, in which PBMCs are processed, that does not comprise use of a point of care system. The PBMC process that does not comprise use of a point of care system comprises 14 separate steps (see FIG. 1, Current PBMC Mfg. Process, 1-14), each of which requires specialized equipment and materials. By comparison and as discussed further herein, a point of care system for processing cells as described herein can be used as a part of a point of care manufacturing process, which process comprises four steps (see FIG. 1, POC Mfg. Process, 1-4), further which steps are performed using a single point of care system as described herein.

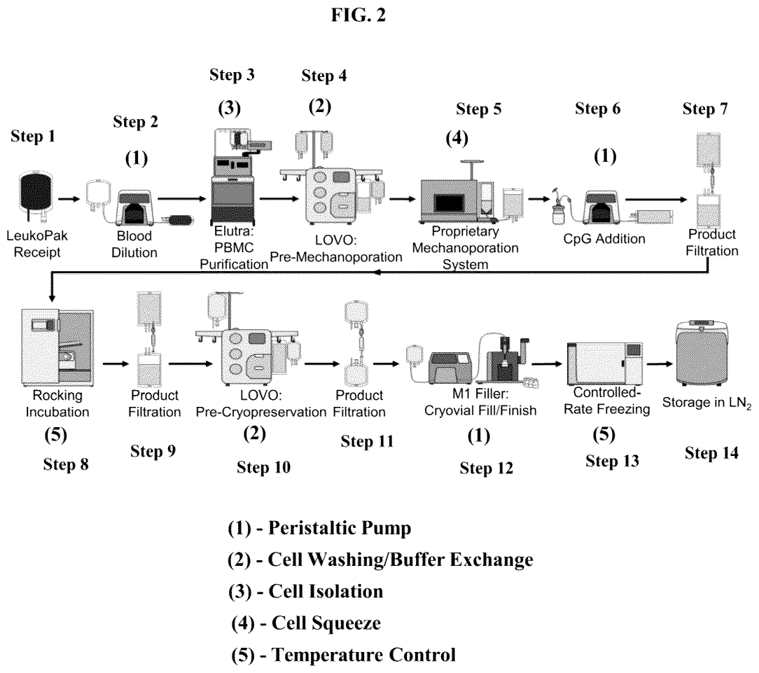

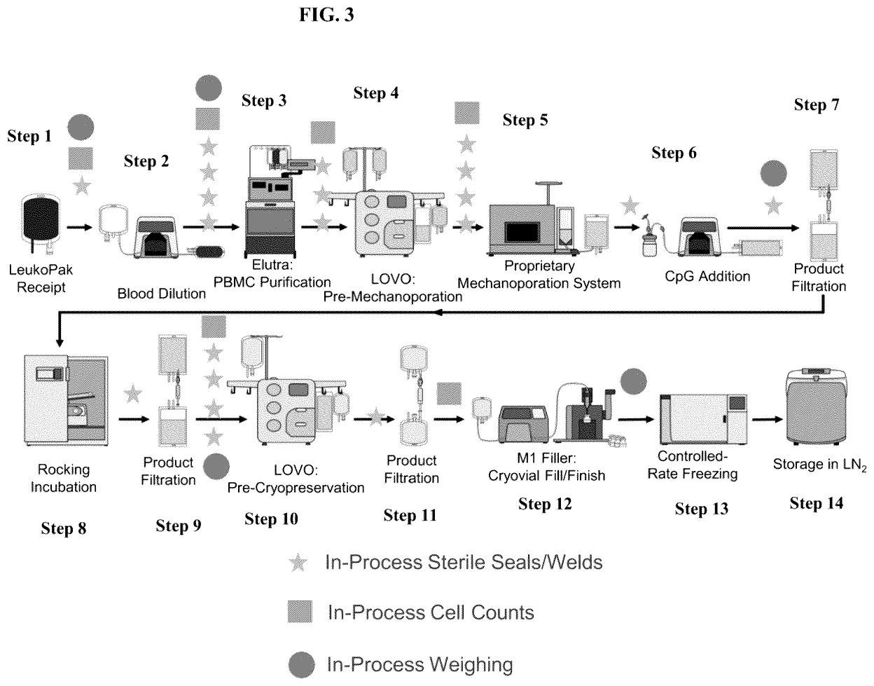

As discussed supra, overall process efficiency is limited by presently available off-the-shelf technology available, and the number of pieces of off-the-shelf technology used to process cells. For instance, referring to FIG. 2-FIG. 3, a method for processing cells that does not comprise use of a point of care system is presented, which process comprises uses of off-the-shelf components. As presented in FIG. 2, numerous pieces of equipment are used to perform the operations of the process, including (1) peristaltic pumps to pump fluid for a given step; (2) a device such as a LOVO device to perform cell washing and buffer exchange operations; (3) a cell isolation device, such as an elutriation device, to perform cell isolation; (4) microfluidic chips and cartridges to perform a cell deformation process; and (5) devices for temperature control, such as rocking incubators and freezers. For instance, as presented in FIG. 2, the following components are used during each of 14 steps of the system and method for processing cells that does not comprise use of a point of care system: at step 1, a container comprising sample, such as a LeukoPak receipt; at step 2, a device for blood dilution; at step 3, a PBMC purification device, such as an elutriation device; at step 4, a LOVO device for pre-mechanoporation; at step 5, a cell-deformation system, such as a mechanoporation system; at step 6, a device for CpG addition; at step 7, a device for product filtration; at step 8, a device for rocking incubation; at step 9, a second product filtration device; a step 10, a LOVO device for pre-cryopreservation operations; at step 11, a third product filtration device; at step 12, a vial or container filling device, such as a cryovial filler; at step 13, a controlled rate freezer; and at step 14, storage, such as storage in a cryopreservation tank.

Furthermore, as presented in FIG. 3, the system for processing cells that does not comprise use of a point of care system contains a number of hidden operations during the 14-step process, including: in-process sterile seals/welds, represented by the stars of FIG. 3; in-process cell counts, represented by the squares of FIG. 3; and in-process weighing, represented by the circles of FIG. 3. For instance, cell counts and weights are required as input parameters for certain pieces of equipment, and each cell count can take up to 30 minutes to perform. Moreover, each step in the 14-step process presented in FIG. 2 and FIG. 3 must be documented, signed, and witnessed, a significant time investment.

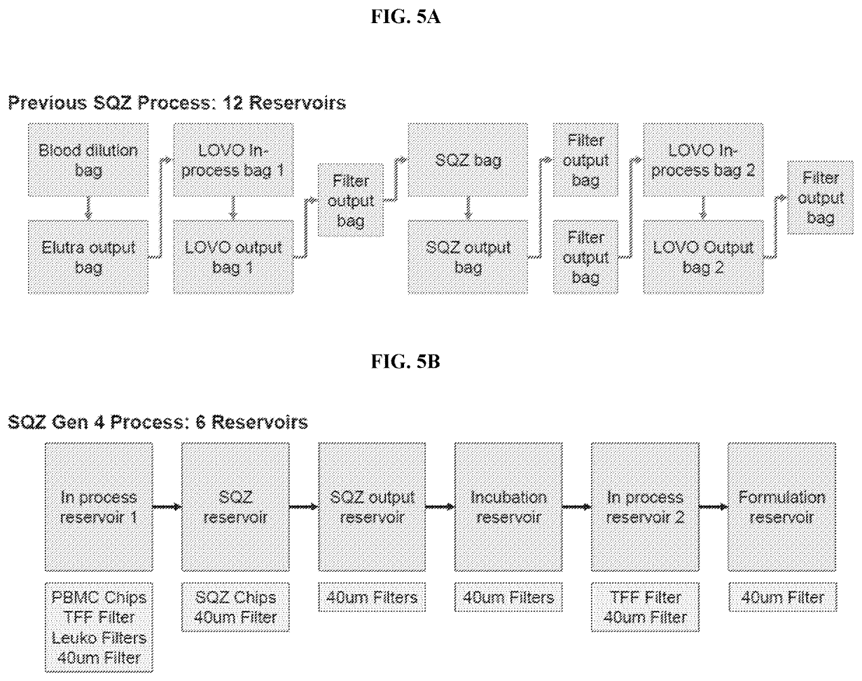

Moreover, a number of the instruments require use of a specialized single use kits that can only be assembled during each manufacturing run. Furthermore, as presented in FIG. 5A, processing cells using the process of FIG. 2 or FIG. 3 can require 12, or more, reservoirs, as compared to just 6 or fewer reservoirs for processing cells using a system for cell processing as described herein (see FIG. 5B).

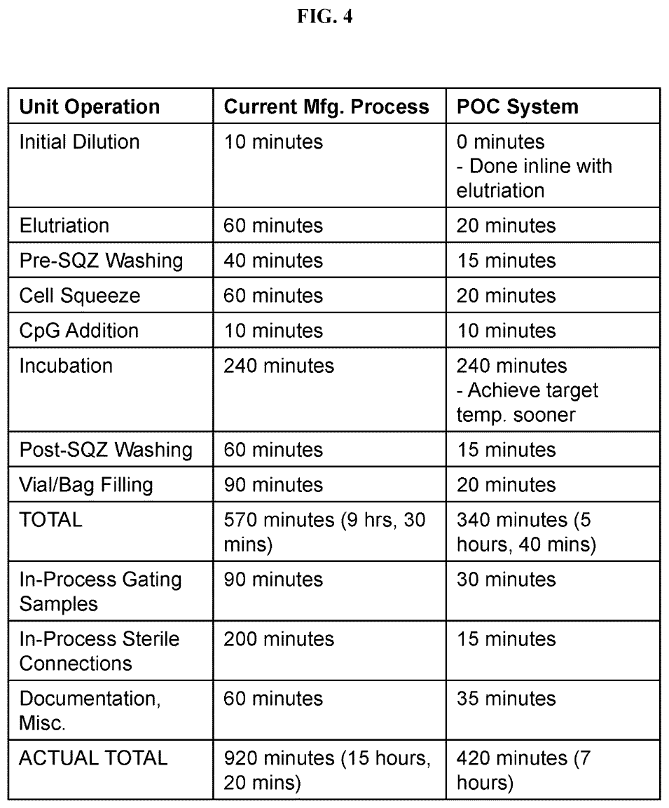

Furthermore, processing cells using the process of FIG. 2 or FIG. 3 can take on the order for 15-18 hours in manufacturing time, if not longer. For instance, FIG. 4 presents a table detailing an average amount of time to complete each step in the cell manufacturing process that does not comprise use of a point of care system, such as presented in FIG. 2 and FIG. 3. For comparison, the amount of time for each corresponding step when using a system for processing cells as described herein is also presented in FIG. 4. As presented in FIG. 4, use of a point of care system for processing cells as described herein can be used to process cells in less time as compared to a process for manufacturing cells that does not comprise use of a point of care system. For instance, the manufacturing process that does not comprise use of a point of care system can be approximately 15 hours and 20 minutes, whereas a manufacturing cells using a system for processing cells as described herein can be approximately 7 hours, a significant time-savings as compared to the current process for manufacturing cells.

b. Point of Care System

As such, the present disclosure is generally related to systems and methods for processing cells, and kits for use with such systems and methods. In some aspects, the system for processing cells can comprise: a suspension preparation subsystem (Zone 1) comprising: a delivery media inlet; a cell isolation device configured to isolate cells, sometimes referred to as a cell separation device; a cell suspension device configured to suspend isolated cells in delivery media thereby creating a cell suspension. In some aspects, the delivery media inlet of Zone 1 can be used to introduce any type of fluid into the system, such as delivery media. In some aspects, a delivery media can be any buffer or fluid compatible with the cells which are to be suspended in the delivery media. In some aspects, Zone 1 can omit the cell isolation device. In some aspects, Zone 1 can comprise one or more additional inlets, such as additional inlets for introducing sample into Zone 1; inlets for introducing buffer into Zone 1; inlets for introducing wash media, such as RPMI or PBS, into Zone 1; inlets for introducing dilution media into Zone 1; and/or inlets for introducing antigen into Zone 1. In some aspects, inlets that are in fluidic communication with Zone 1 can also be in fluidic communication with Zone 2. In some aspects, fluid introduced into the system through any of the inlets of Zone 1 can be temperature-controlled, such that the fluid is introduced into the system at a desired temperature. In some aspects, cells that are introduced into Zone 1 can be isolated from non-desired cell types and aggregates, such as by use of the cell isolation device of Zone 1. In some aspects, cells that are introduced into Zone 1 can be buffer exchanged into delivery media, such as by use of the cell suspension device of Zone 1. In some aspects, the cell suspension device comprises one or more tangential flow filtration (TFF) assemblies. In some aspects, the cell suspension device comprises an one or more ultrasonic standing wave with cross flow chips. In some aspects, the cell suspension device comprises one or more gel filtration chromatography devices. In some aspects, the cell suspension device comprises one or more centrifugal washing devices. In some aspects, the cell suspension device comprises one or more deterministic lateral displacement (DLD) chips. In some aspects, the cell suspension device comprises one or more centrifuge and flexible diaphragm devices. In some aspects, the cell isolation device comprises one or more leukoreduction filters. In some aspects, the cell isolation device comprises one or more spiral inertial separation (SIS) devices. In some aspects, the cell isolation device comprises one or more microfluidic chips. In some aspects, the cell isolation device comprises one or more deterministic lateral displacement (DLD) chips. In some aspects, the cell isolation device comprises one or more elutriation devices. In some aspects, the cell isolation device comprises one or more hydrodynamic microfluidic separation chips. In some aspects, the cell isolation device comprises one or more immunomagnetic cell isolation devices. In some aspects, the cell isolation device comprises one or more acoustic cell processing devices. In some aspects, the cell isolation device comprises one or more fluorescence activated cell sorting (FACS) devices. In some aspects, the cell isolation device comprises one or more microfluidic centrifuge combination chips. In some aspects, the cell isolation device comprises one or more TFF filter assemblies. In some aspects, the cell isolation device comprises one or more dielectrophoresis (DEP) chips. In some aspects, the cell isolation device comprises one or more microfiltration chips. In some aspects, the cell isolation device comprises one or more buoyancy activated cell sorting devices. In some aspects, the cell isolation device comprises one or more sedimentation devices.

In some aspects, the system for processing cells can comprise: a cell-deformation subsystem (Zone 2) in fluid communication with the suspension preparation subsystem, wherein the cell-deformation subsystem comprises: a cell suspension inlet; one or more cell-deforming constrictions configured to cause perturbations in cell membranes of the cells to allow entry of a payload into the cell, thereby creating a cell suspension comprising engineered cells; a preparation vessel configured to cause the cell suspension to flow through the one or more cell-deforming constrictions. In some aspects, the preparation vessel can be a rigid reservoir subassembly. In some aspects, the payload comprises a cargo to be delivered to the cell. For instance, in some aspects, the payload comprises one or more reprogramming factors. In some aspects, the reprogramming factor can comprise a differentiation factor, i.e., any agent that is capable of inducing the differentiation of a cell into a different type of cell. In some aspects, the payload comprises one or more neuron reprogramming factors. In some aspects, the payload comprises a polypeptide, a lipid, a carbohydrate, a small molecule, a metal-containing compound, an antibody, a transcription factor, a nanoparticle, a liposome, a fluorescently tagged molecule, or combinations thereof. In some aspects, the payload comprises one or more nucleic acids. In some aspects, the nucleic acid comprises a DNA, RNA, or both. In certain aspects, DNA comprises a recombinant DNA, a cDNA, a genomic DNA, or combinations thereof. In certain aspects, RNA comprises a siRNA, a mRNA, a miRNA, a lncRNA, a tRNA, a shRNA, a self-amplifying mRNA, or combinations thereof. In some aspects, the preparation vessel can be a rigid reservoir subassembly. In some aspects, Zone 2 can comprise one or more inlets, such one or more inlets for introducing buffer into Zone 2. In some aspects, cell suspension from Zone 1 can enter Zone 2 via the cell suspension inlet. In some aspects, Zone 2 can comprise a container, into which the cell suspension can flow and be stored. In some aspects, the container is temperature-controlled, such as cooled using TECs as described herein. In some aspects, cell suspension comprised in the container can be agitated, such as by using an agitation system described herein. Such agitation can prevent cell settling. In some aspects, the cell deformation process can be effected using an electroporation device, rather than one or more cell-deforming constrictions configured to cause perturbations in cell membranes of the cells to allow entry of a payload into the cell. The electroporation device can be used to allow entry of a payload into a cell, thereby creating a cell suspension comprising engineered cells.

In some aspects, the system for processing cells can comprise: a dilution subsystem (Zone 3) in fluid communication with the cell-deformation subsystem, wherein the dilution subsystem comprises: a cell suspension inlet; an inlet such as for introducing fluids or dry reagents; a container configured to receive the cell suspension comprising engineered cells and to receive buffer that mixes with the cell suspension comprising engineered cells to create a diluted cell suspension. In some aspects, Zone 3 can comprise one or more additional inlets, such one or more additional inlets for introducing fluids, such as buffers, e.g., CpG buffer, into Zone 3. In some aspects, Zone 3 comprises a container, into which the cell suspension comprising engineered cells can flow and be stored. In some aspects, the cell suspension comprising engineered cells in the container of Zone 3 can be diluted with buffer. In some aspects, Zone 3 further comprises one or more scale systems that can be used to measure the amount of buffer added to the cell suspension comprising engineered cells. Such scale systems can be tension load cells compression load cells, or straight bar load cells. In some instances, the scale systems can be in contact with a container, such as the container of Zone 3 comprising the cell suspension comprising engineered cells, and can be used to measure the weight of the container. In some aspects, Zone 3 can further comprise an agitation system, such as described herein. The agitation system can be used, for example, to continuously mix the cell suspension comprising engineered cells and the buffer in the container of Zone 3. In some aspects, Zone 3 further comprises one or more aggregate filters, such as 40 μm aggregate filters, to remove cellular debris and/or aggregates.

In some aspects, the system for processing cells can comprise: an incubation subsystem (Zone 4) in fluid communication with the dilution subsystem, wherein the incubation subsystem comprises: a diluted cell suspension inlet; a container configured to receive the diluted cell suspension; and a plate configured to adjust the temperature of the diluted cell suspension in the container to create an incubated cell suspension. In some aspects, Zone 4 comprises a container, into which the diluted cell suspension can flow and be stored. In some aspects, container of Zone 4 is in contact with a plate, which can be a temperature-controlled plate. In some instances, the temperature-controlled plate can be used to control the temperature of the diluted cell suspension. In some aspects, Zone 4 can further comprise an agitation system, such as described herein. The agitation system can be used, for example, to continuously mix the diluted cell suspension in the container of Zone 4. In some aspects, Zone 4 further comprises one or more aggregate filters, such as 40 μm aggregate filters, to remove cellular debris and/or aggregates

In some aspects, the system for processing cells can comprise: a cell-washing subsystem (Zone 5) in fluid communication with the incubation subsystem, wherein the cell-washing subsystem comprises: an incubated cell suspension inlet; a preservation media inlet; a container configured to receive the incubated cell suspension and to receive preservation media that mixes with the incubated cell suspension in the container thereby suspending the cells in preservation media. In some aspects, Zone 5 comprises one or more additional inlets, such as one or more inlets for introducing buffer, e.g., dimethyl sulfoxide (“DMSO”) buffer, into Zone 5. In some instance, Zone 5 further comprises one or more scale systems that can be used to measure the amount of buffer added to the incubated cell suspension. Such scale systems can be tension load cells, compression load cells, or straight bar load cells. In some instances, the scale systems can be in contact with a container, such as the container of Zone 5 comprising the incubated cell suspension, and can be used to measure the weight of the container. In some aspects, Zone 5 can further comprise one or more cell suspension devices, such as one or more TFF assemblies. Such cell suspension devices can be used for buffer exchange operations, such that the incubated cell suspension is buffer changed into a cryoprotecting preservation media. In some aspects, the cell suspension device comprises one or more tangential flow filtration (TFF) assemblies. In some aspects, the cell suspension device comprises an one or more ultrasonic standing wave with cross flow chips. In some aspects, the cell suspension device comprises one or more gel filtration chromatography devices. In some aspects, the cell suspension device comprises one or more centrifugal washing devices. In some aspects, the cell suspension device comprises one or more deterministic lateral displacement (DLD) chips. In some aspects, the cell suspension device comprises one or more centrifuge and flexible diaphragm devices. In some aspects, Zone 4 can further comprise an agitation system, such as described herein. The agitation system can be used, for example, to continuously mix the contents of the container of Zone 5.

In some instances, the system for processing cells can comprise a container-filling subsystem (Zone 6) in fluid communication with the incubation subsystem, wherein the container-filling subsystem comprises: an inlet configured to receive cells suspended in preservation media; one or more containers configured to receive cells suspended in preservation media; and one or more pumps configured to pump the cells suspended in preservation media into the one or more containers. In some aspects, Zone 6 comprises an additional container into which cells suspended preservation media flow and are stored prior to filling the one or more containers. In some aspects, Zone 6 further comprises one or more scale systems, such as described herein. The scale systems can be in contact with each of the one or more containers and can be used to measure the weight of each of the one or more containers. In some aspects, Zone 6 further comprises an aggregate filter, such as a 40 μm aggregate filter, which can be used to remove cellular debris and/or aggregates prior to filling the one or more containers. In some aspects, Zone 6 can further comprise an agitation system, such as described herein. The agitation system can be used, for example, to continuously mix the contents of the additional container of Zone 6. In some aspects, each of the containers of Zone 6 can be in contact with a respective temperature-controlled plate so as to control the temperature of the contents of a given container.

In some aspects, the system is sterile and configured to be used in a non-sterile location. Such a feature is advantageous and presents a significant advantage over manufacturing processes that do not comprise use of a point of care system. For instance, manufacturing processes for cell processing that do not comprise use of a point of care system generally occur in at least one clean room facility, which represents a significant cost to set up and to maintain as well as requiring a significant amount of space in which to perform the cell processing. Contrastingly, the system for processing cells as described herein can in some aspects be a sterile system that can be used in a non-sterile environment, i.e., not a clean room environment. Such as system could be used in many locations that do not have the capability of providing a clean room for cell processing. As such, a system for processing cells as described herein can be used, for instance, at a hospital or at another point of care location.

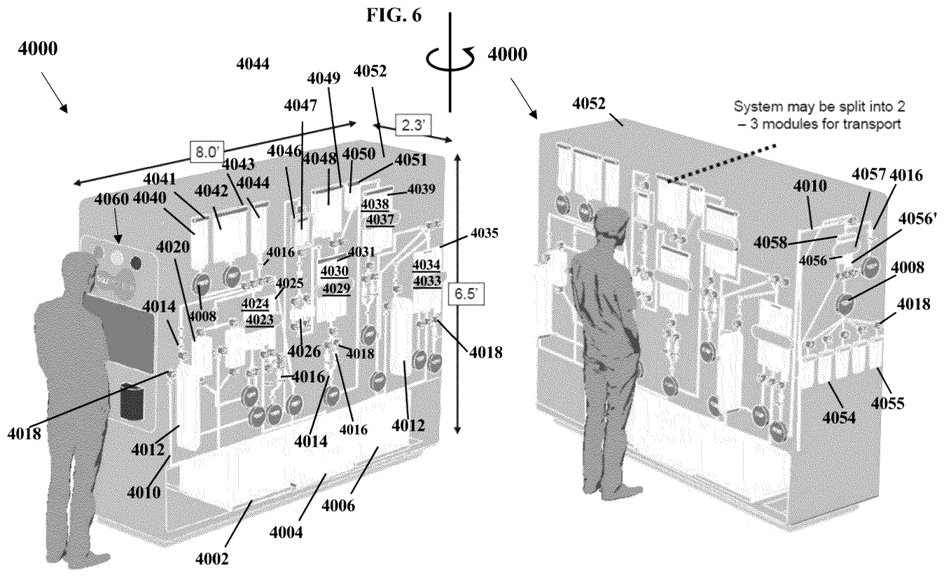

Referring to FIG. 6, in some aspects, a point of care system comprises point of care system 4000. In some instances, frame 4052 can comprise frame 6000 of FIG. 8. For instance, frame 4052 can comprise plates on which containers rest, such as temperature-controlled plates; agitation systems for mixing the contents of containers throughout the system, as described further infra; scale systems, such as tension load cells, compression load cells, or straight bar load cells, for measuring the weight of containers throughout the system; fastening elements, such as hooks, which can be used to releasably couple containers or various other components to the frame; filter clips, which can be used to releasably couple components to the frame; valves to control fluid flow throughout the system; pumps, such as peristaltic pumps, to pump fluid within subsystems and between subsystems; one or more casters for moving the frame and/or assembled system; a graphical user interface (GUI) which can be used by an operator to control the system; and backlights for illuminating containers throughout the system. In some aspects, fastening elements throughout the system can be any type of connector. For example, the fastening elements can be a hook, a hook-and-loop fastener, a temporary adhesive, a tie, or pins. In some aspects, the temperature-controlled plates can be used to control the temperature of the contents of a respective container in contact with a given plate. In some instances, frame 4052 of the system is designed to be split into at least 2 modules, such as represented by the dashed line of the rotated view of system 4000. Such splitting of the frame of the system can provide for ease of transport of the point of care system. In some aspects, the overall size of the assembled frame 4052 of the system is about 8.0 feet long, about 6.5 feet high, and about 2.3 feet deep. In some aspects, point of care system 4000 comprises container 4040, which can comprise a fluid for introduction into the system, e.g., antigen in buffer, in some instances. Container 4040 can be releasably coupled to the system, such as by coupling container 4040 to fastening element 4041, which can be a hook and which can also function as a part of a mechanism for weighing container 4040, e.g., fastening element 4041 can be a part of scale system. In some aspects, container 4040 is fluidically connected to Zone 1 by tubing, such as tubing 4010, and the fluid contained within container 4040 is pumped from the bag through the tubing by a pump, such as a pump 4008. Tubing 4010 can be PVC tubing, such as di (2-ethylhexyl) phthalate (DEHP) free PVC tubing, and fluid can flow through tubing 4010. Point of care system 4000 can further comprise container 4042, which can comprise a buffer. Container 4042 can be releasably coupled to the system, such as by coupling container 4042 to fastening element 4043, which fastening element can also function as a part of a scale system for weighing container 4042. In some aspects, container 4042 is fluidically connected to Zone 1 by tubing and the fluid contained within container 4042 is pumped from the container through the tubing by a pump. Point of care system 4000 can further comprise container 4044, which can comprise sample for processing, such as an input blood sample. Container 4044 can be releasably coupled to the system, such as by coupling container 4044 to a fastening element 4045, which fastening element 4045 can be a hook and can also function as a part of a scale system for weighing container 4044. In some aspects, container 4044 is fluidically connected to Zone 1 by tubing, such as tubing 4010, and the fluid contained within container 4044 flows by gravity through an aggregate filter, such as aggregate filter 4016, prior to entering Zone 1. In some instances, aggregate filter 4016 can be a 40 μm aggregate filter, which can filter particles of sizes greater than 40 μm, such as cellular aggregates or debris. In some instances, the filter diameter can be altered for a given process. For instance, the filter size can be about 10 μm, about 20 μm, about 30 μm, about 40 μm, or about 50 μm. In some aspects, the filter material of aggregate filter 4016 is selected for a given process. In some instances, the filter material of aggregate filter 4016 can be mixed cellulose esters, cellulose acetate, coated cellulose acetate, hydrophilic polytetrafluoroethylene (PTFE), hydrophobic PTFE, nylon, or polycarbonate.

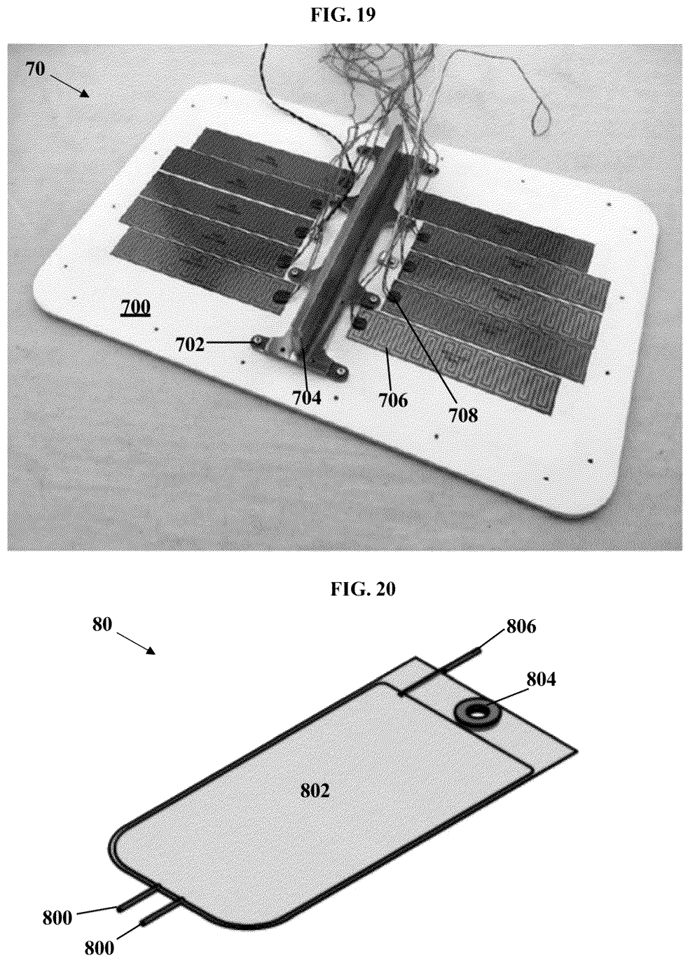

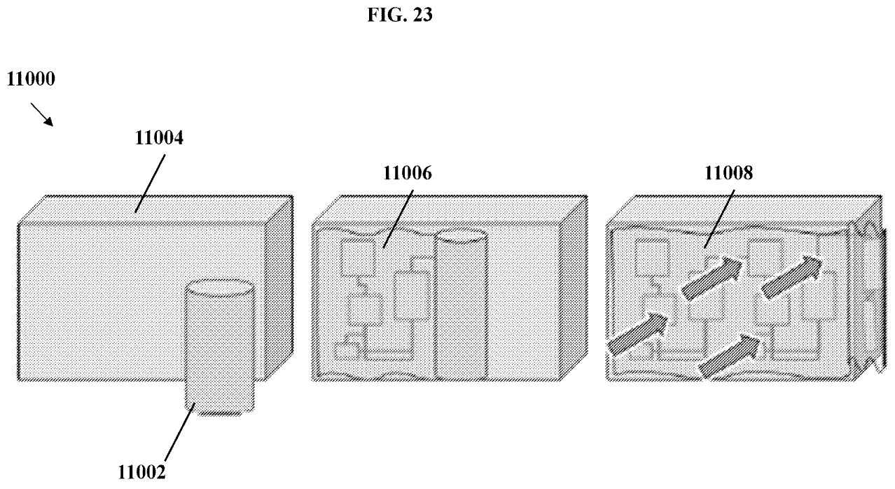

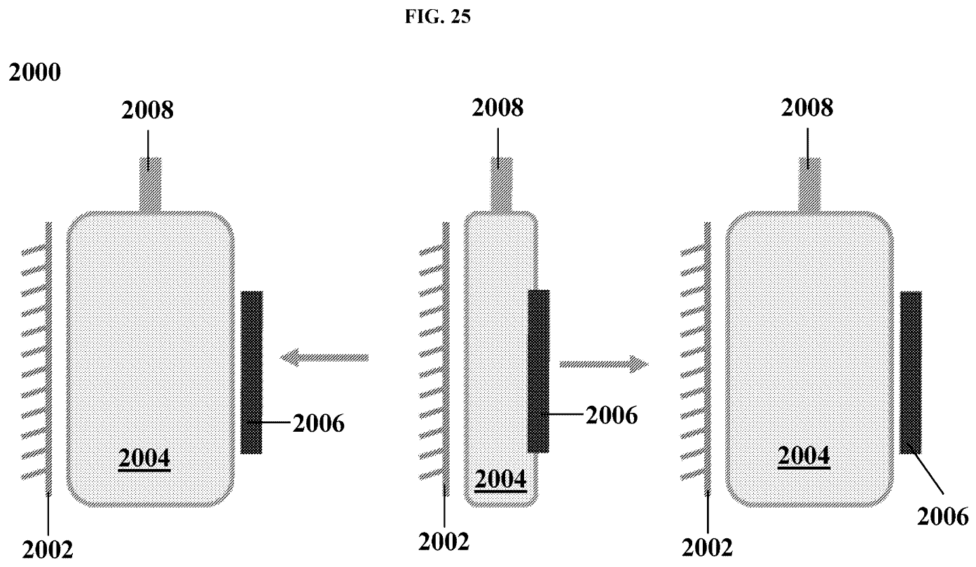

In some aspects, sample flowing from container 4044 through aggregate filter 4016 flows into container 4024. In some aspects, container 4024 is in contact with a plate, such as a plate 4025. A plate such as plate 4025 can be a temperature-controlled plate, such as to heat or cool the sample in the container. Mechanisms for heating and cooling plates are presented in FIG. 19 and FIGS. 23A-23B, respectively, and are discussed further infra. In some aspects, an agitation system 4023 is in contact with container 4024. In some aspects, agitation system 4023 can be used to homogenize the sample in container 4024. Agitation system 4023 can be agitation system 2000 of FIG. 25, a rocking plate, or a shaking plate. In some aspects, container 4024 is fluidically connected via tubing to a cell suspension device 4012, such as a tangential flow filtration (TFF) filter assembly, which TFF filter assembly is releasably coupled to the frame of the system. The cell suspension device 4012, such as TFF filter assembly, is fluidically connected to valves, such as valve 4018, to regulate flow into and out of the cell suspension device 4012, such as a TFF filter assembly. The cell suspension device 4012, such as a TFF filter assembly, can be used to suspend cells in a desired fluid, such as delivery media. Furthermore, prior to entering the cell suspension device 4012, fluid passes through one or more air filters, such as air filter 4014, to remove air, such as in the form of air bubbles, from the fluid. In some aspects, cell suspension device 4012 is in fluid communication with one or more cell isolation devices, such as cell isolation device 4020. In some aspects, cell isolation devices 4020 comprise one or more microfluidic chips. In some aspects, cell isolation devices 4020 comprise one or more SIS devices. In some aspects, cell isolation devices 4020 comprise one or more leukoreduction filters. In some aspects, cell isolation devices 5020 comprise one or more elutriation devices. In some aspects, cell isolation devices 4020 are in fluidic communication with container 4024 by tubing. In some aspects, Zone 1 can omit the cell isolation devices 4020.

In some aspects, container 4024 is fluidically connected by tubing to container 4047 of Zone 2. Fluid can be pumped from container 4024 by a pump, and the fluid can pass through an aggregate filter to remove additional cellular debris or aggregate that may have formed during Zone 1 processing, such as by pumping fluids throughout Zone 1 or during a cell isolation process, for example, prior to entering container 4047. In some aspects, container 4047 is releasably coupled to the frame by a fastening element, such as by a hook, a hook-and-loop fastener, temporary adhesive, tie, or pins. In some aspects, container 4047 is in contact with a plate, such as plate 4046. Plate 4046 can be temperature-controlled, such as heated, cooled, or kept at a relatively constant temperature, to control the temperature of the sample in container 4047. Container 4047 is in fluid communication with an aggregate filter by tubing, through which sample can pass prior to entering cell deformation devices 4026. In some aspects, cell deformation devices 4026 are in fluidic communication with container 4030 of Zone 3 through tubing 4010. In some aspects, container 4030 is releasably coupled to the system by a fastening element, such as by a hook. In some aspects, container 4030 can be in contact with plate 4031, which plate can be temperature-controlled, such as heated, cooled, or kept at a relatively constant temperature. In some aspects, the temperature-controlled plate can be a plate as presented in FIG. 19. In some aspects, the temperature-controlled plate is used to control the temperature of the contents of the container. In some aspects, container 4030 is in contact with agitation system 4029. In some aspects, container 4048 is in fluidic communication with container 4030 by tubing, which container 4048 can comprise buffer. Container 4048 can be releasably coupled to the frame 4042, such as by a fastening element 4049.

In some aspects, container 4038 is in fluidic communication with container 4050 by tubing, which container can comprise buffer. Container 4050 can be releasably coupled to the frame 4042, such as by fastening element 4051. In some aspects, container 4030 is in fluid communication with aggregate filters 4016. Aggregate filters 4016 can be used to filter cellular debris and aggregates that may have accumulated during cell processing using the system. In some aspects, fluid can flow from container 4030 through the aggregate filters and can then be pumped to container 4038 of Zone 4, which is in fluidic communication with the aggregate filters and container 4030 via tubing.

In some aspects, container 4038 is releasably coupled to frame 4052, such as by fastening element 4039, which can be in some instances a hook. In some instances, fastening element 4039 can also be used a part of a scale system to weigh container 4038. In some aspects, the scale system is a tension load cell, a compression load cell, or a straight bar load cell. In some aspects, container 4038 is in contact with agitation system 4037. Agitation system 4037 can be, for example, agitation system 2000 of FIG. 25, a rocking plate, or a shaking plate. In some aspects, container 4038 is in fluidic communication with container 4050 by tubing. In some instances, container 4038 is in fluidic communication with aggregate filters, such as 40 μm aggregate filters that can be used to remove cellular debris and/or cellular aggregates. In some aspects, fluid can flow from container 4038 through the cell aggregate filters and subsequently flow through tubing into container 4034 of Zone 5.

In some aspects, container 4034 is in fluidic communication with a second cell suspension device 4012, such as a second TFF filter assembly. In some aspects, the cell suspension device 4012, such as the second TFF filter assembly, can be used to suspend the cells in a cryopreservation media. In some aspects, the second cell suspension device 4012 is releasably coupled to the frame of the system. In some aspects, container 4034 is in fluidic communication with container 4056 of Zone 6 by tubing. In some aspects, container 4034 is in contact with agitation system 4033. Agitation system 4033 can be used to mix the contents of the container. Container 4034 can further rest on plate 4035 in some instances, which plate can be a temperature-controlled plate.

In some aspects, container 4056 is in contact with agitation system 4057. In some aspects, container 4056 is releasably coupled to the frame of the system by a fastening element, such as by a hook. In some aspects, container 4056 is in contact with a plate, such as a temperature-controlled plate. In some aspects, container 4056 is in contact with backlight 4056′. Container 4056 is further in fluidic communication with container 4054, which can serve as containers for the processed cells. In some aspects, containers 4054 can rest on a scale system 4055 to measure the weight of the bags containing the processed cells. In some aspects, point of care system 4000 further comprises waste containers for buffer, 4002, for general system waste, 4004, and for DMSO buffer, 4006. Each of these containers is in fluidic communication with the subsystems, e.g., Zones 1-6, of the system via tubing. In some aspects, each of the waste containers is in contact with a scale system, wherein the scale system can comprise a tension load cell or a compression load cell.

In some aspects, one or more of Zones 1-6 can be omitted from the system. For instance, Zone 4 can be omitted from the system, and Zone 3 can be used to perform the functions otherwise performed by Zone 4. For example, plate 4031 of Zone 3 can be a temperature-controlled plate, such that container 4030 can be incubated by using plate 4031. In some aspects, Zone 5 can be omitted from the system, and Zone 1 an be used to perform the functions otherwise performed by Zone 5. For instance, the cell suspension device of Zone 1, such as a TFF filter assembly can be used to suspend the cells in cryopreservation media, as can be performed by the components of Zone 5. In some aspects, the tubing of each Zone is fabricated independently of a second, different Zone, such that a first Zone can be connected to a second, different zone via connecting the tubing of the first and second Zone. Such manufacturing allows for the Zones to be assembled in different orientations and using different numbers of Zones, as may be desired for a given process.

In some aspects, the point of care system can comprise a temperature control subsystem. The temperature control subsystem can be used to perform the functions otherwise performed by the dilution subsystem (Zone 3) and incubation subsystem (Zone 4). For instance, the temperature control subsystem can comprise a container for receiving cell suspension comprising engineered cells from the cell deformation subsystem (Zone 2) and one or more inlets for introducing fluids and/or dry reagents to container comprising the cell suspension. The fluids and/or dry reagents can be mixed with the cell suspension, such as by use of an agitation system, to create a diluted cell suspension. The temperature control system can further comprise a plate configured to adjust the temperature of the diluted cell suspension in the container to create an incubated cell suspension. In some aspects, the temperature control system can be fluidically connected to a cell deformation subsystem (Zone 2) and also fluidically connected to cell-washing subsystem (Zone 5).

In some aspects, Zone 3 and Zone 4 can be merged. For instance, a filtration recirculation loop could be used when merging Zone 3 and Zone 4. In some aspects, a first container of Zone 2, which is in contact with a temperature-controlled plate, can be merged with Zone 1. For instance, temperature-controlled plate can be added to Zone 1 to contact a container, and the first container of Zone 2 can be eliminated. In some aspects, the cell suspension device of Zone 1, such as a TFF filter assembly, could be used to perform a cell suspension operation performed by a cell suspension device of Zone 5. As such, the cell suspension device of Zone 5 could be eliminated. For instance, the cell suspension device of Zone 1 could be rinsed prior to reuse for a cell-washing operation. A branch from Zone 5 to Zone 1 could be added, such as by tubing, to connect Zone 5 and Zone 1 directly. In some instances, the cell suspension device of Zone 1 can comprise more than one different filters. In some instances, Zone 5 can be eliminated from the system when the cell suspension device of Zone 1 is used for a cell-washing operation. In some aspects, Zone 4 could be merged with Zone 5. For instances, a heating element and a recirculating filter loop could be added to Zone 5 so as to perform incubation and cell-washing within Zone 5.

In some aspects, point of care system 4000 can further comprise a graphical user interface (GUI) system 4060. A user can interface with the GUI to control the point of care system, such as to plan and execute cell processing runs using the point of care system.

In some aspects, an enclosure can be created over the working area of a point of care system. For instance, disposable kits can be installed on the frame of a point of care system, and subsequently a cover, such as a sash, could be used to close the front of the point of care system. Such closure of the system can help in maintaining sterile conditions in some instances. In some aspects, a point of care system can further comprise an environmental monitoring system.

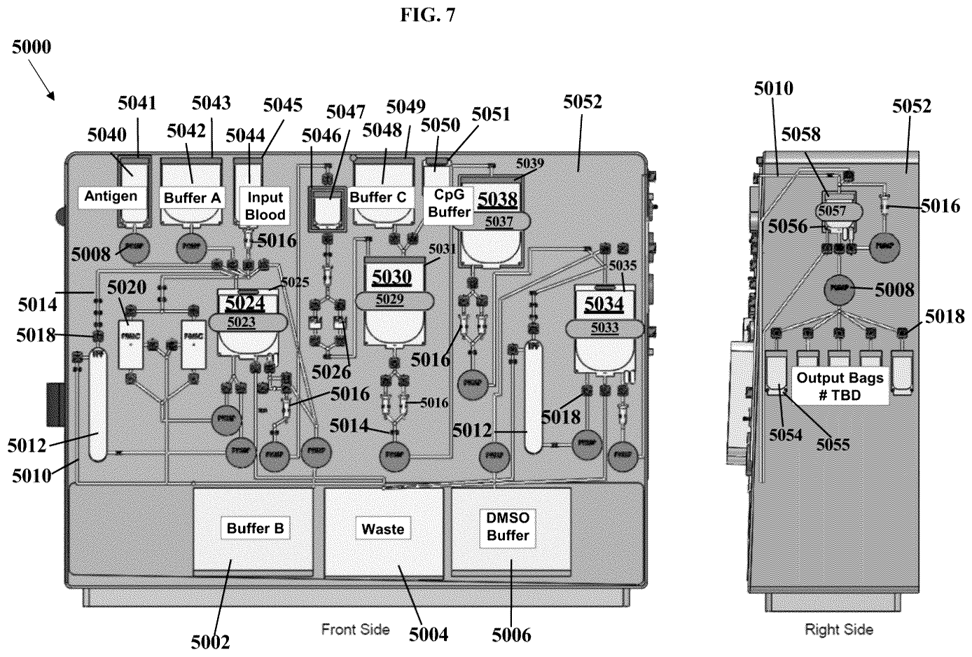

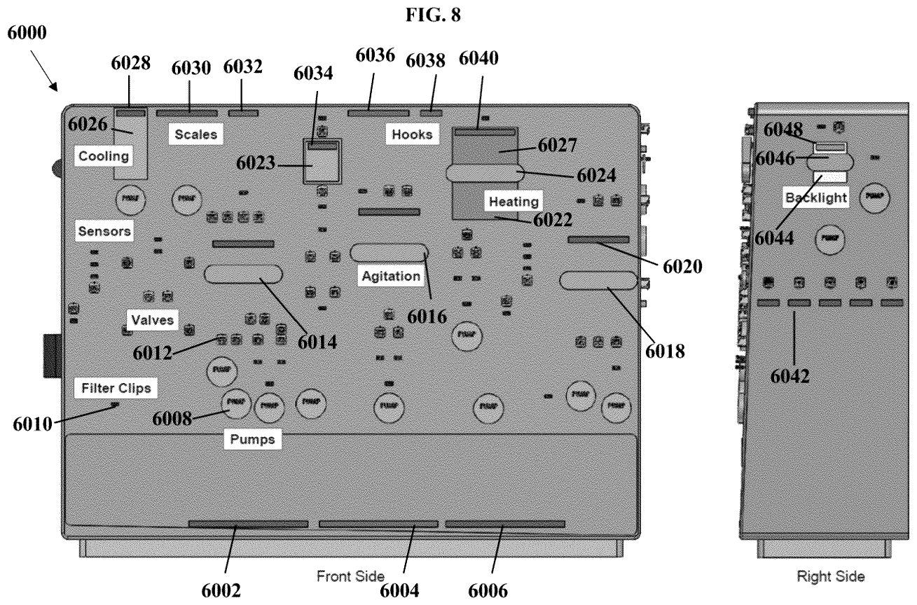

In some aspects, the point of care system comprises point of care system 5000 as presented in FIG. 7. In some aspects, frame 5052 of point of care system 5000 can comprise plates on which containers rest, such as temperature-controlled plates; agitation systems for mixing the contents of containers throughout the system, as described further infra; scale systems, such as tension load cells, compression load cells, or straight bar load cells, for measuring the weight of containers throughout the system; fastening elements such as hooks, which can be used to releasably couple containers or various other components to the frame; filter clips, which can be used to releasably couple components to the frame; valves to control fluid flow throughout the system; pumps, such as peristaltic pumps, to pump fluid within subsystems and between subsystems; one or more casters for moving the frame and/or assembled system; a graphical user interface (GUI) which can be used by an operator to control the system; and backlights for illuminating containers throughout the system. In some aspects, fastening elements throughout the system can be any type of connector. For example, the fastening elements can be a hook, a hook-and-loop fastener, a temporary adhesive, a tie, or pins. In some aspects, the temperature-controlled plates can be used to control the temperature of the contents of a respective container in contact with a given plate. In some aspects, frame 5052 of point of care system 5000 can comprise frame 6000 of FIG. 8.

In some aspects, point of care system 5000 comprises container 5040, which can comprise antigen in some instances. Container 5040 can be releasably coupled to the system, such as by coupling container 5040 to fastening element 5041, which fastening element can be a hook and can also function as a part of a scale system for weighing container 5040. In some aspects, container 4040 is connected to Zone 1 by tubing, such as tubing 5010, and the fluid contained within container 5040 is pumped from the bag through the tubing by a pump, such as pump 5008. Tubing 5010 can be PVC tubing, and fluid can flow through tubing 5010.

Point of care system 5000 can further comprise container 5042, which can comprise a buffer. Container 5042 can be releasably coupled to the system, such as by coupling container 5042 to fastening element 5043, which fastening element can be a hook and can also function as a part of a mechanism for weighing container 5042. In some aspects, container 5042 is connected to Zone 1 by tubing, such as tubing 5010, and the fluid contained within container 5042 is pumped from the container through the tubing by a pump, such as a pump 5008.

Point of care system 5000 can further comprise container 5044, which can comprise sample for processing, such as an input blood sample. Container 5044 can be releasably coupled to the system, such as by coupling container 5044 to a fastening element 5045, which fastening element 5045 can be a hook and can also function as a part of scale system for weighing container 5044. In some aspects, container 5044 is connected to Zone 1 by tubing, such as tubing 5010, and the fluid contained within container 5044 flows by gravity through an aggregate filter 5016, prior to entering Zone 1. In some instances, aggregate filter 5016 can be a 40 μm aggregate filter, which can filter particles of sizes greater than 40 μm, such as cellular aggregates or debris. In some instances, the filter diameter can be altered for a given process. For instance, the filter size can be about 10 μm, about 20 μm, about 30 μm, about 40 μm, or about 50 μm. In some aspects, the filter material of aggregate filter 5016 is selected for a given process. In some instances, the filter material of aggregate filter 5016 can be mixed cellulose esters, cellulose acetate, coated cellulose acetate, hydrophilic polytetrafluoroethylene (PTFE), hydrophobic PTFE, nylon, or polycarbonate. In some aspects, sample flowing from container 5044 through aggregate filter 5016 flows into container 5024. In some aspects, container 5024 is in contact with a plate, such as a plate 5025. A plate such as plate 5025 can be temperature-controlled, such as to heat or to cool the sample in the container. In some aspects, an agitation system 5023 is in contact with container 5024. Agitation system 5023 can be agitation system 2000 of FIG. 25, a rocking plate, or a shaking plate. In some aspects, agitation system 5023 can be used to homogenize the sample in container 5024. In some container 5024 is fluidically connected via tubing to a cell suspension device 5012, such as a tangential flow filtration (TFF) filter assembly, which TFF filter assembly is releasably coupled to the frame of the system. Cell suspension device 5012 is fluidically connected to valves, such as valve 5018, to regulate flow into and out of the cell suspension device. The cell suspension device, such as a TFF filter assembly, can be used to suspend cells in a desired fluid, such as delivery media. Furthermore, prior to entering the cell suspension device 5012, fluid passes through one or more filters, such as air filter 5014 to remove air, such as in the form of air bubbles, from the fluid. In some aspects, the cell suspension device 5012 is in fluid communication with one or more cell isolation devices, such as cell isolation device 5020. In some aspects, cell isolation devices 5020 comprise one or more SIS devices. In some aspects, the cell isolation devices 5020 are SIS devices. In some aspects, the cell isolation devices are used to separate PBMC cells from other cell types. In some aspects, cell isolation devices 5020 comprise one or more leukoreduction filters. In some aspects, cell isolation devices 5020 comprise one or more elutriation devices. In some aspects, cell isolation devices 5020 are in fluidic communication with container 5024 by tubing. In some aspects, Zone 1 can omit the cell isolation devices 5020.

In some aspects, containers 5024 are fluidically connected by tubing to container 5047 of Zone 2 for a cell deformation process to occur. Fluid can be pumped from container 5024 by a pump, and the fluid can pass through an aggregate filter, prior to entering container 5047. In some aspects, container 5047 is releasably coupled to the frame by a fastening element, such as by a hook. In some aspects, container 5047 is in contact with plate 5046. Plate 5046 can be temperature-controlled, such as heated, cooled, or kept at a relatively constant temperature, to control the temperature of the sample in container 5047. In some aspects, the temperature-controlled plate can be a plate as presented in FIG. 19. In some aspects, the temperature-controlled plate is used to control the temperature of the contents of the container. Container 5047 is in fluid communication with an aggregate filter by tubing, through which sample can pass prior to entering cell deformation devices 5026. In some aspects, cell deformation devices 5026 are in fluidic communication with container 5030 of Zone 3 through tubing 5010. In some aspects, container 5030 is releasably coupled to the system by a fastening element, such as by a hook, a hook-and-loop fastener, a temporary adhesive, a tie, or pins. In some aspects, container 5030 can rest on a plate, such as plate 5031, which can be temperature-controlled. Moreover, container 5030 can be in contact with a plate, which plate can be temperature-controlled, such as heated, cooled, or kept at a relatively constant temperature. In some aspects, container 5030 is in contact with agitation system 5029. In some aspects, container 5048 is in fluid communication with container 5030 by tubing, which container 5048 can comprise buffer. Container 5048 can be releasably coupled to the frame 5042, such as by a fastening element 5049.

In some aspects, container 5038 is in fluidic communication with container 5050 by tubing, which container can comprise buffer. Container 5050 can be releasably coupled to the frame 5042, such as by fastening element 5051. In some aspects, container 5030 is in fluid communication with aggregate filters 5016. In some aspects, fluid can flow from container 5030 through the aggregate filters and can then be pumped to container 5038 of Zone 4, which is in fluidic communication with the aggregate filters and container 5030 via tubing.

In some aspects, container 5038 is releasably coupled to frame 5052, such as by fastening element 5039. In some instances, fastening element 5039 can be a hook and can also be used a part of a scale system to weigh container 5038. In some aspects, container 5038 is in contact with agitation system 5037. In some aspects, container 5038 is in fluidic communication with container 5050 by tubing. In some instances, container 5038 is in fluidic communication with aggregate filters 5016. Aggregate filters 5016 can be used to filter cellular debris and aggregates that may have accumulated during cell processing using the system. In some aspects, fluid can flow from container 5038 through the cell aggregate filters and subsequently flow through tubing into container 5034 of Zone 5.

In some aspects, container 5034 is in fluidic communication with a second cell isolate device 5012, such as a second TFF filter assembly, which is releasably coupled to the frame of the system. In some aspects, container 5034 is in fluidic communication with container 5056 of Zone 6 by tubing. In some aspects, container 5034 is in contact with agitation system 5033. Container 5034 can further contact plate 5035 in some instances, which plate can be a temperature-controlled plate.

In some aspects, container 5056 is in contact with agitation system 5057. In some aspects, container 5056 is releasably coupled to the frame of the system by a fastening element, such as by a hook. In some aspects, container 5056 is in contact with a plate, such as a temperature-controlled plate. In some aspects, container 5056 is in contact with backlight 4056. Container 5056 is further in fluidic communication with container 5054, which can serve as containers for the processed cells. In some aspects, containers 5054 can be in contact with scale system 5055 to measure the weight of the bags containing the processed cells.

In some aspects, point of care system 5000 further comprises waste containers for buffer, 5002, for general system waste, 5004, and for DMSO buffer, 5006. Each of these containers is in fluidic communication with subsystems, i.e., Zones 1-6, of the system via tubing. In some aspects, each of the waste containers is in contact with a scale system, wherein the scale system can comprise a tension load cell or a compression load cell.

In some aspects, one or more of Zones 1-6 can be omitted from the system. For instance, Zone 4 can be omitted from the system, and Zone 3 can be used to perform the functions otherwise performed by Zone 4. For example, plate 5031 of Zone 3 can be a temperature-controlled plate, such that container 5030 can be incubated by using plate 5031. In some aspects, Zone 5 can be omitted from the system, and Zone 1 an be used to perform the functions otherwise performed by Zone 5. For instance, the cell suspension device of Zone 1, such as a TFF filter assembly can be used to suspend the cells in cryopreservation media, as can be performed by the components of Zone 5. In some aspects, the tubing of each Zone is fabricated independently of a second, different Zone, such that a first Zone can be connected to a second, different zone via connecting the tubing of the first and second Zone. Such manufacturing allows for the Zones to be assembled in different orientations and using different numbers of Zones, as may be desired for a given process.

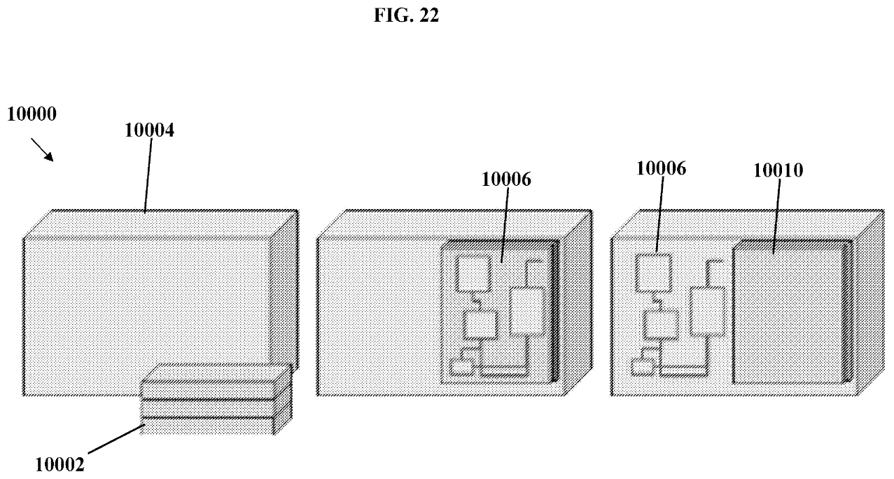

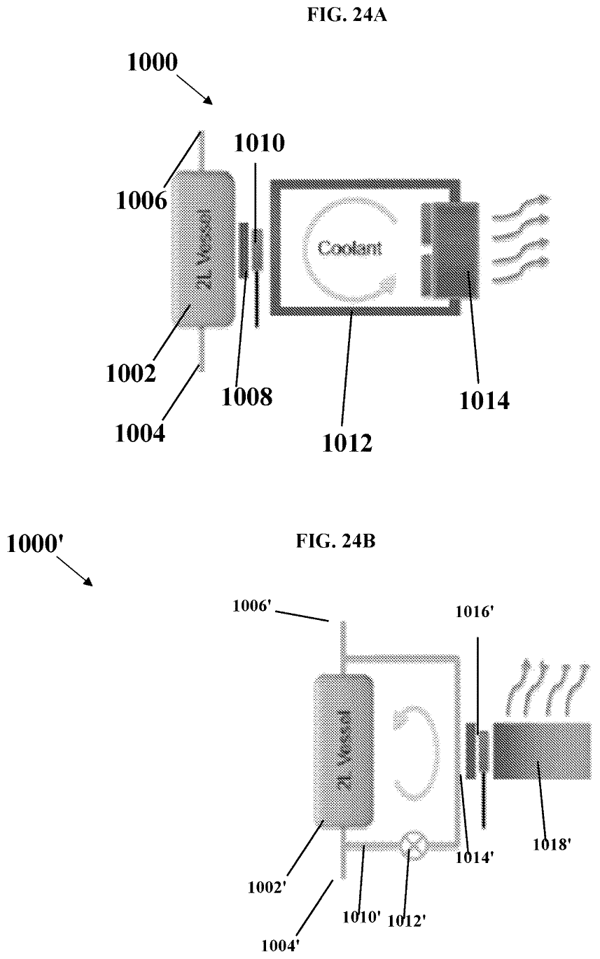

In some aspects, frame of the system for processing cells can comprise various different components built in to the frame of the system, such as those presented in FIG. 8. As presented in FIG. 8, the frame of the system can comprise in some instances heating stations 6022 and 6027, which heating stations can be heated plates (see FIG. 22, for instance). System 6000 can further comprise cooling stations 6023 and 6026, which can be in-line TEC liquid cooling systems (see FIG. 24A-FIG. 24B, for instance). In some aspects, system 6000 can comprise agitation systems 6014, 6016, 6018, 6024, and 6046, such as the agitation system presented in FIG. 25. In some aspects, system 6000 can comprise waste container holders, such as 6002, 6004, and 6006. In some aspects, each of waste container holders 6002, 6004, and 6006 can comprise a scale system for weighing a given waste container. In some aspects, system 6000 comprises filter clips, which can be used to releasably couple components to the frame 6000. In some aspects, frame 6000 comprises pumps 6008 to pump fluid through the system. In some aspects, frame 6000 comprises valves 6012 to control fluid flow throughout the system. In some aspects, frame 6000 comprises fastening elements 6020, 6028, 6030, 6032, 6034, 6036, 6038, 6040, 6042, and 6048, which can in some instances be hooks. Such fastening elements can be used to releasably couple containers to frame 6000. Moreover, such fastening elements, such as hooks, can be used as a part of a scale system to measure the weight of containers releasably coupled to a given fastening element. In some aspects, frame 6000 comprises backlight 6044, which can be used to illuminate sample in a container, which container can be releasably coupled to fastening element 6048. In some aspects, fastening element throughout the system can be any type of connector. For example, the fastening element can be a hook, a hook-and-loop fastener, a temporary adhesive, a tie, or pins.

In some aspects, the point of care system can comprise one or more TEC cooling loops, such as presented in FIG. 24A and FIG. 24B. Referring now to FIG. 24A, TEC cooling loop 1000 can comprise container 1002, which comprises inlet 1004 and 1006. In some aspects, container 1002 is in contact with plate 1008, which is in contact with plate 1010. In some aspects, plate 1010 is in contact with cooling loop tubing 1012, which is connected to cooling station 1014 via tubing 1012. Referring now to FIG. 24B, TEC cooling loop 1000′ can comprise container 1002′, which comprises inlet 1006′ and 1004′, which can be connected to tubing 1010′. In some aspects, tubing 1010′ is further connected to 1012′, and tubing 1010′ is in contact with plate 1014′. Plate 1014′ is in contact with plate 1016′, which is in contact with cooling unit 1018′. In some aspects, a temperature-controlled plate can be in contact with a TEC cooling loop.

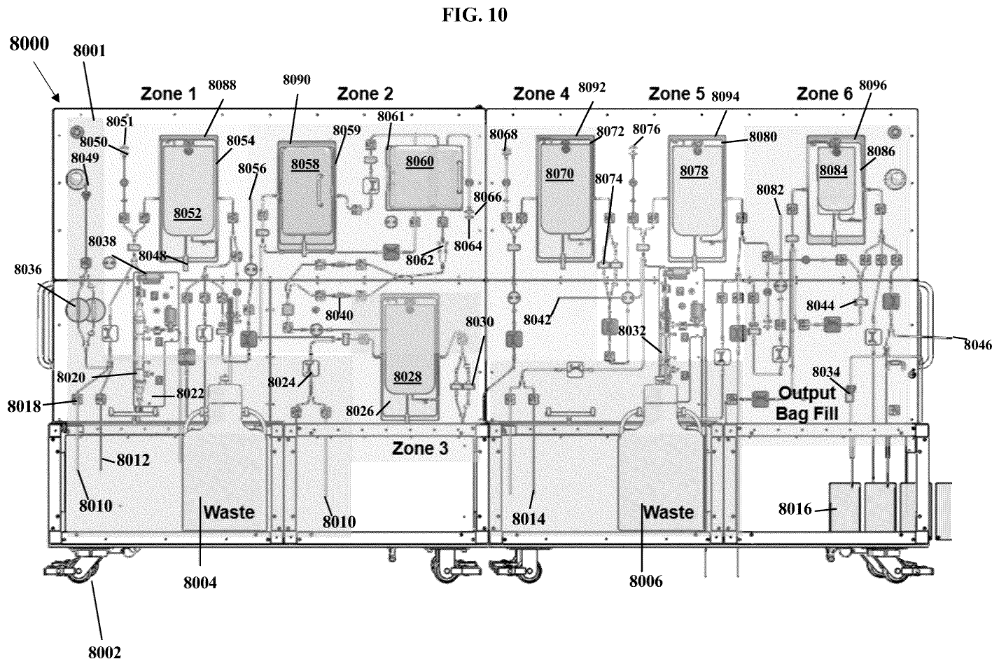

In some aspects, the point of care system for processing cells can comprise system 8000 as presented in FIG. 10. In some aspects, frame 8001 of point of care system 8000 can comprise plates on which containers rest, such as temperature-controlled plates; agitation systems for mixing the contents of containers throughout the system, as described further infra; scale systems, such as tension load cells, compression load cells, or straight bar load cells, for measuring the weight of containers throughout the system; fastening elements, e.g., hooks, which can be used to releasably couple containers or various other components to the frame; filter clips, which can be used to releasably couple components to the frame; valves to control fluid flow throughout the system; pumps, such as peristaltic pumps, to pump fluid within subsystems and between subsystems; one or more casters for moving the frame and/or assembled system; a graphical user interface (GUI) which can be used by an operator to control the system; and backlights for illuminating containers throughout the system. In some aspects, fastening elements throughout the system can be any type of connector. For example, the fastening elements can be a hook, a hook-and-loop fastener, a temporary adhesive, a tie, or pins. In some aspects, the temperature-controlled plates can be used to control the temperature of the contents of a respective container in contact with a given plate.

In some aspects, system 8000 comprises casters 8002. Casters 8002 can allow an operator to move system 8000. In some aspects, system 8000 comprises inlets 8049, 8051, and 8056, which inlets can be used to introduce fluids, e.g., sample, e.g., buffer, e.g., delivery media, into Zone 1 of system 8000. In some aspects, the inlets can have air filters 8050 fluidically connected to the inlet. Air filters can be used to remove air, such as in the form of air bubbles, from fluids introduced into the system. In some aspects, Zone 1 of system 8000 further comprises cell isolation devices 8036, which can be leukoreduction filters. In some aspects, cell isolation devices 8036 comprise one or more microfluidic chips. In some aspects, cell isolation devices 8036 comprise one or more SIS devices. In some aspects, cell isolation devices 8036 comprise one or more leukoreduction filters. In some aspects, cell isolation devices 8036 comprise one or more elutriation devices. In some aspects, Zone 1 can omit the cell isolation device. In some aspects, Zone 1 further comprises one or more cell suspension devices, such as TFF filter assembly 8020 which can be in contact with plate 8022. Cell suspension device 8020 can be used to suspend cells in a desired buffer, such as delivery media. Cell suspension device 8020 is further connected to pressure gauges 8038 to monitor pressure. Cell suspension device 8020 is releasably coupled to the frame of the system. In some aspects, Zone 1 further comprises waste outlets 8010.

In some aspects, Zone 1 further comprises container 8052, which is in contact with plate 8054 and further which can be releasably coupled to the frame of the system by fastening element 8088, which can in some instances be a hook. In some instances, plate 8054 can be a temperature-controlled plate, such as heated, cooled, or maintained at a relatively constant temperature. In some aspects, Zone 1 further comprises valves, such as valve 8018, to control movement of fluid in Zone 1. In some aspects, container 8052 is in fluid communication with 8048. In some aspects, container 8052 is in fluid communication with the cell suspension device via tubing 8012. In some aspects, container 8052 is in fluidic communication with container 8058 of Zone 2 via tubing.

In some aspects, container 8058 of Zone 2 is in contact with plate 8059, which can be a temperature-controlled plate, such as heated, cooled, or kept at a relatively constant temperature. In some aspects, container 8058 is releasably coupled to frame 8001 via a fastening element 8090, which can be a hook. In some aspects, container 8058 is in fluidic communication with container 8060 via tubing. In some aspects, container 8060 is in contact with plate 8061, which can be a temperature-controlled plate, such as heated, cooled, or maintained at a relatively constant temperature. In some aspects, container 8060 is further in fluidic communication with inlet 8064, which inlet is connected to air filters 8066. Moreover, container 8060 is fluidically connected to aggregate filter 8062 via tubing, and this is further fluidically connected to cell deformation devices 8040. In some aspects, the cell deformation devices 8040 comprise electroporation devices. In some aspects, cell deformation devices 8040 comprise one or more microfluidic chips comprising one or more cell-deforming constrictions configured to cause perturbations in cell membranes of the cells to allow entry of a payload into the cell. In some aspects, cell deformation devices 8040 are fluidically connected to container 8028 of Zone 3 via tubing. In some aspects, valves, such as valve 8024, can direct fluid flow into waste outlets, such as waste outlets 8010.

In some aspects, container 8028 is in contact with plate 8026, which can be a temperature-controlled plate, such as heated, cooled, or maintained at a relatively constant temperature. In some aspects, container 8028 is further in fluidic communication with aggregate filters 8030 via tubing. Aggregate filters 8030 can be used to filter cellular debris and aggregates that may have accumulated during cell processing using the system. In some aspects, aggregate filters are fluidically connected to container 8070 of Zone 4 via tubing. In some aspects, container 8070 is releasably coupled to frame 8001 by fastening element 8092, which can in some instances be a hook. In some aspects, container 8070 is in contact with plate 8072, which can be a temperature-controlled plate, such as heated, cooled, or maintained at a relatively constant temperature. In some aspects, container 8070 is in fluidic communication with inlet 8068 via tubing. In some aspects, inlet 8068 can be used to introduce buffer into Zone 4. In some aspects, container 8070 is in fluidic communication with aggregate filters 8074 via tubing. In some aspects, aggregate filters 8074 are in fluidic communication with container 8078 of Zone 5 via tubing. In some aspects, container 8078 of Zone 5 is in contact with plate 8080, which can be a temperature-controlled plate. In some aspects, container 8078 is releasably coupled to frame 8001 by fastening element 8094, which can in some instances be a hook. In some aspects, container 8078 is in fluidic communication with inlet 8042 via tubing. In some aspects, container 8078 is in fluidic communication with a cell suspension device, such as TFF filter assembly 8032, via tubing. Such cell suspension devices can be used for buffer exchange operations, such that the incubated cell suspension is buffer changed into a cryoprotecting preservation media. In some aspects, inlet 8076 is fluidically connected to a cell suspension device, such as TFF filter assembly 8032, via tubing. In some aspects, the cell suspension device, such as TFF filter assembly 8032, is releasably coupled to the frame 8001 of the system. In some aspects, waste from Zone 5 can flow through tubing to waste outlet 8014.

In some aspects, container 8078 is fluidically connected to container 8084 of Zone 6 via tubing. In some aspects, container 8084 is in contact with plate 8086, which can be a temperature-controlled plate. In some aspects, container 8084 is releasably coupled to frame 8001 by fastening element 8096, which can in some instances be a hook. In some aspects, container 8084 is in fluidic communication with inlets 8082 and 8046 via tubing. In some aspects, container 8084 is in fluidic communication with aggregate filter 8044 via tubing. In some aspects, container 8084 is fluidically connected to a valve, such as a valve 8034, which regulates the flow of fluid into sample containers 8016. In some aspects, waste can flow from via tubing from a given zone into a waste container, such as waste containers 8004 and 8006. In some aspects, waste from two or more different zones flow into the same waste container. In some aspects, waste from two or more zones each flow into their own respective waste containers. In some aspects, waste is grouped by type and flows into a container for a given type of waste. For instance, systems 8000 can comprise dedicated waste containers for buffer, for general system waste, 8004, and for DMSO buffer, 8006. In some aspects, each of the waste containers is in contact with a scale system, wherein the scale system can comprise a tension load cell or a compression load cell.

In some aspects, the point of care system comprises one or more heated plates, such as presented in FIG. 19. Referring now to FIG. 19, heated plate 70 comprises plate 700, which can be coupled to attachment bar 704 by fasteners 702, such as screws, bolts, nuts, washers, retaining rings, and the like. Plate 700 is further connected by connectors 708, such as a wire connector, temperature ring connector, or the like, to heating units 706, which can be used to control the temperature of the plate.

In some aspects, the system for processing cells can be an automatic system for processing cells. For instance, the system can be operated with minimal operator interaction with the system, e.g., in one or more of the zones. In some aspects, an operator can load cells into the system, such as by releasably coupling a LeukoPak to the frame of the system, and the operator can then initiate cell processing by using a GUI of the system. In some aspects, the operator can use the GUI to program cell processing runs, to view the progress of the run, to view errors in the system, and/or to view results of the cell processing run.

In some aspects, the system for processing cells comprises tubing to fluidically connect components of the system. For instance, the tubing can be PVC tubing, such as DEHP free PVC tubing. In some aspects, the tubing can be flexible plastic tubing. In some aspects, the tubing can be rigid plastic tubing. In some aspects, the tubing can be metal tubing.

As discussed supra, the system for processing cells as described herein can produce containers comprising processed cells in about 5 hours to about 7 hours, which is a significant time savings over manufacturing processes that do not comprise use of a point of care system. Such a time savings can allow for cells to be harvested from a patient, processed using the system, and administered to the patient within less than a day, a significant time improvement and significant benefit to the patient receiving the processed cells. Moreover, as discussed supra, in some aspects, the system is sterile and configured to be used in a non-sterile location. This feature is particularly advantageous as such as system could be used in many locations that do not have the capability of providing a clean room for cell processing. As such, a system for processing cells as described herein can be used, for instance, at a hospital or at another point of care location that does not have a clean room or otherwise sufficiently sterile environment for performing cell processing that does not comprise use of a point of care system.

i. Subsystems

As described supra, the system for processing cells can comprise one or more subsystems, which subsystems include a suspension preparation subsystem (Zone 1), a cell deformation subsystem (Zone 2), a dilution subsystem (Zone 3), an incubation subsystem (Zone 4), a cell washing subsystem (Zone 5), and a container filling subsystem (Zone 6). In some instances, the system for processing cells can comprise a temperature control subsystem, which in some instances can be used in place of the dilution subsystem and incubation subsystem, as described further infra.

### 1. Suspension Preparation Subsystem (Zone 1)

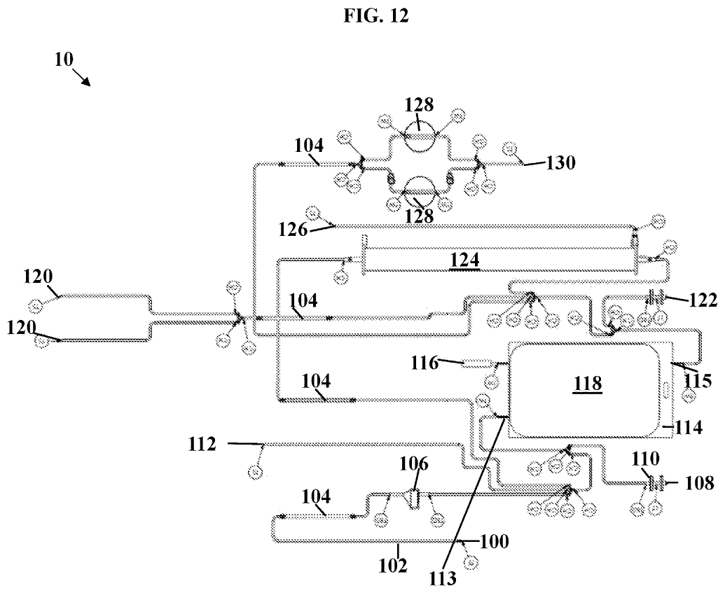

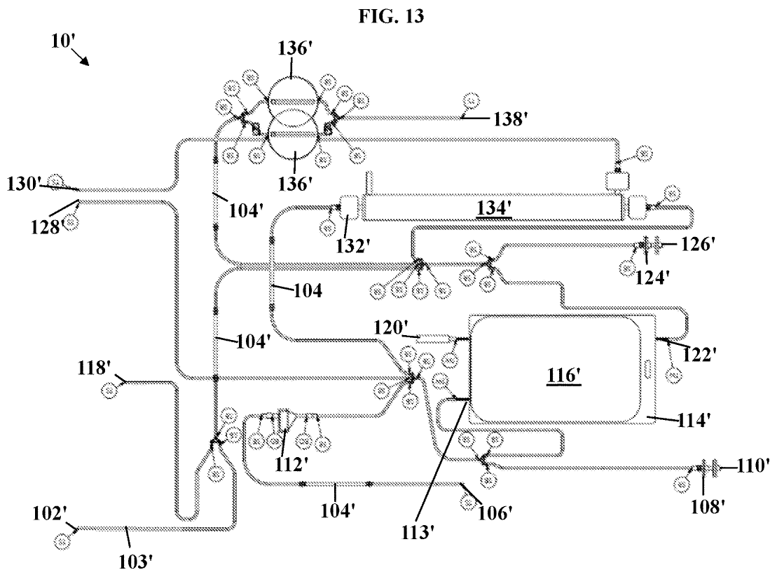

In some aspects, a system for processing cells as described herein comprises a suspension preparation subsystem. In some aspects, the suspension preparation subsystem is designed for processing PBMCs. In some aspects, the suspension preparation subsystem is designed for processing RBCs. In some aspects, the suspension preparation subsystem can be used to isolate desired cells from cellular debris, aggregates, and undesired cell types, and further to suspend cells in a desired delivery media, such as by using a cell suspension device to perform a buffer exchange operation on a cell suspension. In some aspects, the suspension preparation subsystem comprises the subsystem presented in FIG. 12. In some aspects, the suspension preparation subsystem comprises the subsystem presented in FIG. 13.

Referring now to FIG. 12, in some aspects, the suspension preparation subsystem 10 comprises cell suspension inlet 130. Cell suspension inlet 130 can be fluidically connected via tubing 102 to a cell isolation device 128, such as one or more leukoreduction filters. The cell isolation device 128, such as leukoreduction filters, can be fluidically connected via tubing 102 to pump tubing subassembly 104. In some aspects, Zone 1 can omit the cell isolation device. In some aspects, the suspension preparation subsystem comprises delivery media inlet 126, which can be used to introduce delivery media into the suspension preparation subsystem. Delivery media inlet 126 can be fluidically connected via tubing 102 to cell suspension device 124, such as tangential flow filtration (TFF) filter assembly. In some aspects, the suspension preparation subsystem further comprises wash media inlet 122, which is coupled to one or more filters 110, such as 0.2 μm air filters. Such a filter removes particles greater than 0.2 μm and can be used to remove air, such as air in the form of air bubbles, from a fluid. In some aspects, the suspension preparation subsystem further comprises a container 118, such as a bag, which container contacts a plate, such as plate 114, which is in some instances a temperature-controlled plate. Container 118 can have an inlet and an outlet, such as inlet 115 and outlet 114 which are connected to tubing. Plate 114 is further fluidically connected to sample bulb 116. In some aspects, fluid, such as sample, can flow into sample bulb 116 from container 118. In some instances, the sample bulb can be welded off, and the fluid in the sample bulb can be analyzed. In some aspects, the suspension preparation subsystem further comprises outlets fluidically connected to waste containers via tubing 102, such as outlets 120 and 112. In some aspects, the suspension preparation subsystem further comprises dilution media inlet 108 which is coupled to one or more filters 110, such as one or more 0.2 μm air filters. In some aspects, the suspension preparation subsystem further comprises aggregate filter 106 which is positioned at the end of the suspension preparation subsystem and is connected to a pump tubing subassembly 104, which is fluidically connected via tubing 102 to a cell deformation subsystem 20 via outlet 100. In some aspects, the aggregate filter 106 can be a 40 μm aggregate filter, which can filter particles of sizes greater than 40 μm, such as cellular aggregates or debris. In some instances, the filter diameter can be altered for a given process. For instance, the filter size can be about 10 μm, about 20 μm, about 30 μm, about 40 μm, or about 50 μm. In some aspects, the filter material of aggregate filter 4016 is selected for a given process. In some instances, the filter material of aggregate filter 4016 can be mixed cellulose esters, cellulose acetate, coated cellulose acetate, hydrophilic polytetrafluoroethylene (PTFE), hydrophobic PTFE, nylon, or polycarbonate. Further referring to FIG. 12, TW represents tube weld; CH represents cyclohexanone; LT represents adhesive; and TS represents seal.

Referring now to FIG. 13, in some aspects, the suspension preparation subsystem 10′ comprises cell suspension inlet 138′. Cell suspension inlet 138′ can be fluidically connected via tubing 103′ to a cell isolation device 136′, such as one or more leukoreduction filters. In some aspects, Zone 1 can omit the cell isolation device. The cell isolation device 136′, such as leukoreduction filters, can be fluidically connected via tubing 103′ to pump tubing subassembly 104′. In some aspects, the suspension preparation subsystem comprises wash media inlet 110′, which is coupled to filters 108′, such as air filters, and tubing 103′. In some aspects, the suspension preparation subsystem comprises delivery media inlet 126′, which is coupled to filters 124′, such as air filters, and tubing 103′. Delivery media inlet 126′ can be fluidically connected via tubing 103′ to cell suspension device 134′, such as tangential flow filtration (TFF) filter assembly. In some instances, the cell suspension device 134′ is coupled to pressure gauges, such as pressure gauges 132′. In some aspects, the suspension preparation subsystem 10′ further comprises a container 116′, such as a bag, which container contacts a plate, such as plate 114′, which is in some instances a temperature-controlled plate. Container 116′ can have an inlet and an outlet, such as inlet 122′ and outlet 113′ which are connected to tubing. Plate 114′ is further connected to sample bulb 120′. In some aspects, fluid, such as sample fluid, can flow into sample bulb 120′ from container 116′. In some instances, the sample bulb can be welded off, and the fluid in the sample bulb can be analyzed. In some aspects, the suspension preparation subsystem further comprises outlets fluidically connected to waste containers via tubing 102, such as outlets 130′, 128′, 118′, and 102′. In some aspects, container 116 is further fluidically connected via tubing to aggregate filter 112′, which is further fluidically connected via tubing to outlet 106′, which is an outlet to Zone 2. In some aspects, the aggregate filter 106′ can be a 40 μm aggregate filter, which can remove cellular debris and/or aggregates. Further referring to FIG. 13, TW represents tube weld; SB represents solvent bond; and TS represents seal.

In some aspects, the suspension preparation subsystem (Zone 1) components of FIG. 12 and FIG. 13 can be releasably coupled to a frame of a POC system, such as presented in FIG. 6-FIG. 10. In some aspects, the suspension preparation subsystem (Zone 1) components can further be used to perform the cell-washing performed by the cell-washing subsystem (Zone 5) as Zone 1 comprises the components needed to perform such a process.

In some aspects, the suspension preparation subsystem further comprises a wash media inlet. In some aspects, the suspension preparation subsystem further comprises a dilution media inlet. In some aspects, the suspension preparation subsystem further comprises an elutriation system configured to perform the cell isolation operation on the cells. In some aspects, the suspension preparation subsystem further comprises a leukoreduction filter system configured to perform the cell isolation operation on the cells. In some aspects, the suspension preparation subsystem further comprises a tangential flow filtration system configured to perform a buffer exchange operation. In some aspects, the suspension preparation subsystem further comprises at least one outlet configured to be coupled to at least one container for receiving cells suspended in delivery media. In some aspects, the container is a bag comprising at least one inlet and at least one outlet.

In some aspects, the suspension preparation subsystem comprises a scale system configured to weigh a container. The scale system can be a part of the frame of the system. In some aspects, the scale system comprises a tension load cell. In some aspects, the scale system comprises a compression load cell. In some aspects, the scale system comprises a straight bar load cell. In some aspects, the scale system can be used to measure the volume of a fluid, such as a buffer, added to a container. Such measurements can be performed when, for instance, diluting cells in Zone 3 or when filling output containers in Zone 6 to ensure that each container receives the same volume of product.

In some aspects, the suspension preparation subsystem further comprises an agitation system comprising a platform configured to contact a container and to rock the container up and down. In some aspects, the suspension preparation subsystem further comprises an agitation system comprising a plate configured to contact a container and to move in and out while contacting the container. For instance, an example of such an agitation system is presented in FIG. 25. Referring to FIG. 25, container 2004 with an inlet 2008, such as a bag, rests on plate 2002. The agitation system further comprises plate 2006, which plate is movable and contacts container 2004. While contacting container 2004, plate 2006 can move in and out, thereby agitating the bag. In some aspects, the agitation system is a part of the frame of the system.

In some aspects, the suspension preparation subsystem further comprises one or more pumps configured to move fluid within the subsystem. In some aspects, the suspension preparation subsystem further comprises one or more pumps configured to move fluid between fluidically connected subsystems. The pumps can be a part of the frame of the system.

In some aspects, the suspension preparation subsystem is capable of removing serum from sample that is introduced into the subsystem. In some aspects, the suspension preparation subsystem is capable of removing plasma from sample that is introduced into the subsystem. For instance, the cell suspension device of the suspension preparation subsystem, such as a TFF filter assembly, can remove plasma and/or serum during a cell suspension process using the TFF filter assembly. In some aspects, cell suspension using the cell suspension device of Zone 1, e.g., TFF filter assembly, can remove about 2-fold, about 3-fold, about 4-fold, about 5-fold, about 6-fold, about 7-fold, about 8-fold, about 9-fold, or about 10-fold more serum and/plasma as compared to a sample that was not subjected to cell suspension.

In some aspects, the suspension preparation subsystem further comprises one or more of the following components: tube fittings, connectors, clamps, a sampling bulb, a carboy, and an air filter.

### 2. Cell Deformation Subsystem (Zone 2)

In some aspects, a system for processing cells as described herein comprises a cell deformation subsystem. In some aspects, the cell deformation subsystem can be used to introduce a payload into cells. In some aspects, the cell-deformation subsystem further comprises a pressurization system configured to generate pressure to force the cell suspension through the one or more cell-deforming constrictions. For instance, a schematic representation of a cell being forced through a cell-deforming constriction is presented in FIG. 34. Referring now to FIG. 34, a cell 17000 can be passed through cell-deforming constriction 17002, thereby allowing payload, such as payload 17004, to center the cell.

In some aspects, the cell-deformation subsystem further comprises an electroporation device for performing a cell deformation process. In some aspects, the cell-deformation subsystem further comprises a temperature control system comprising a heated plate configured to control a temperature of the cell suspension. In some aspects, the cell-deformation subsystem further comprises at least one outlet configured to be coupled to at least one container for receiving a cell suspension or a cell suspension comprising engineered cells.

In some aspects, the container is a bag such as presented in FIG. 20. Referring now to bag 80 of FIG. 20, bag 80 can comprise outlet tubing 800. In some aspects, bag 80 can further comprise inlet tubing 806 and grommet 804. In some aspects, bag film 802 can comprise ultra-low density polyethylene/ethylene vinyl alcohol (ULDPE/EVOH). In some aspects, the container is a plastic container, a screw top cryo vial, a bag, a sealed vial, a glass container, a plastic bottle, or a glass container.

In some aspects, the cell-deformation subsystem further comprises an agitation system comprising a platform in contact with the at least one container configured to rock the container up and down. In some aspects, wherein the agitation system comprising a platform in contact with the at least one container is configured to rock the container up and down to agitate the cell suspension to promote homogeneity of the cell suspension or the cell suspension comprising engineered cells. In some aspects, the cell-deformation subsystem further comprises an agitation system comprising a plate configured to contact the container and to move in and out while contacting the container, such as, for example, presented in FIG. 25. In some aspects, the agitation system is a part of the frame of the system.

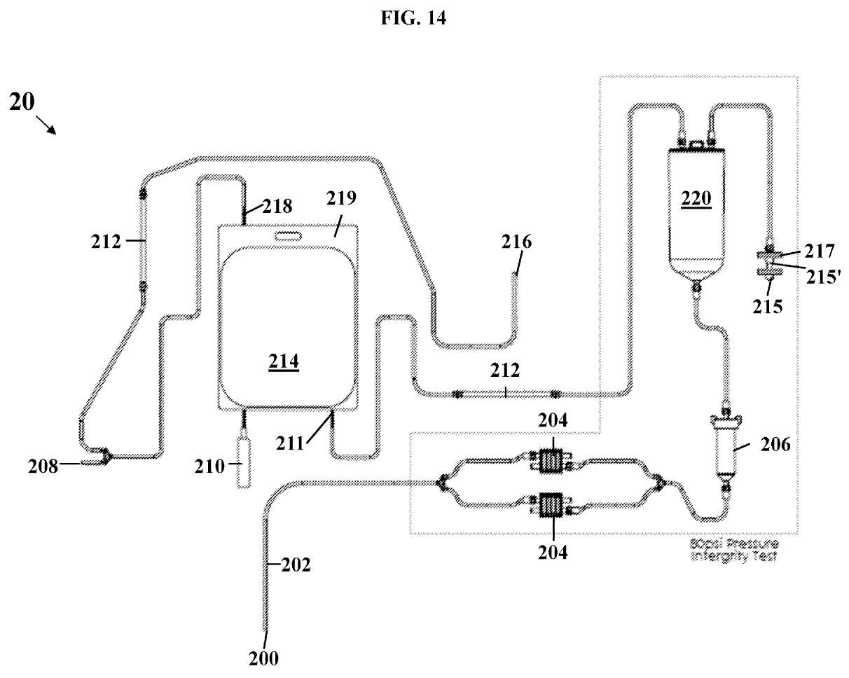

In some aspects, the cell deformation subsystem comprises cell deformation subsystem 20 presented in FIG. 14. In some aspects, cell deformation subsystem comprises cell suspension inlet 208 which is fluidically connected via tubing 202 to the suspension preparation subsystem 10. In some aspects, the cell suspension inlet 208 can further be fluidically connected to outlet 218 via tubing 202, which is configured to deliver the cell suspension to container 214 which contacts plate 219. In some aspects, plate 219 can be temperature-controlled, such as heated, cooled, or maintained at a relatively constant temperature. In some aspects, plate 219 can cooled, such as by using a TEC liquid cooling system. In some aspects, the TEC liquid cooling system can comprise a TEC liquid cooling system 1000 as presented in FIG. 24A-FIG. 24B. In some aspects, container 218 can comprise inlet 218 and outlet 211 which are coupled to tubing and to the container. In some aspects, plate 219 further comprises sample bulb 210. In some aspects, fluid, such as sample, can flow into sample bulb 210 from container 218. In some instances, the sample bulb can be welded off, and the fluid in the sample bulb can be analyzed. In some aspects, the cell deformation subsystem can comprise buffer inlet 216, which can be fluidically connected to pump tubing subassembly 212 via tubing 202. In some aspects, the cell deformation subsystem can comprise end cap 215, which end cap is releasably coupled to inlet 215′ and which end cap can be removed following coupling to the frame of the system. Inlet 215′ can in fluidic communication with filters 217, which can be 0.2 μm air filters. Buffer inlet 215′ can further be fluidically connected to preparation vessel 220 via tubing 202, which can be a preparation vessel 220, such as a rigid reservoir assembly. Preparation vessel 220 can further be fluidically connected via tubing 202 to barrel filter subassembly 206, which can be a 40 μm barrel filter subassembly. In some aspects, barrel filter subassembly 206 can be fluidically connected via tubing 202 to cell deformation devices 204. In some aspects, cell deformation devices 204 are fluidically connected via tubing 202 and outlet 200 to a dilution subsystem, such as dilution subsystem 30.

Cell deformation devices 204 can include microfluidic devices and in some instances cartridges to house the microfluidic devices. In some aspects, the cell deformation devices comprise one or more cell-deforming constrictions configured to cause perturbations in cell membranes of the cells to allow entry of a payload into the cell. In some aspects, the perturbations are caused by flowing the cell suspension through the cell-deforming constrictions under high pressure, such as can be introduced by using cell deformation subsystem 20. In some aspects, the cell deformation device, such as microfluidic devices and cartridges, can be any of the cell deformation devices described by PCT/US2018/066295 and PCT/US2020/026891, each of which is hereby incorporated by reference in its entirety.

In some aspects, the cell suspension can be passed through an electric field generated by at least one electrode after passing through a constriction of a microfluidic chip of a cell deformation device. In some aspects, the electric field assists in delivery of payload to cells of the cell suspension. For example, a combination of a cell-deforming constriction and an electric field can be used to deliver payload, such as a plasmid, into the cells, e.g., the cell nucleus. In some aspects, one or more electrodes are in proximity to the cell-deforming constriction of a microfluidic chip to generate an electric field. In some aspects, the electric field is between about 0.1 kV/m to about 100 MV/m, or any number or range of numbers therebetween. In some aspects, an integrated circuit is used to provide an electrical signal to drive the electrodes. In some aspects, the cells of the cell suspension are exposed to the electric field for a pulse width of between about 1 ns to about 1 s, a period of between about 100 ns to about 10 s, or any time or range of times therebetween.

In some aspects, cell deformation devices 204 can further include electroporation devices, such as one or more electrodes positioned such that cells of the cell suspension are exposed to an electric field generated by the one or more electrodes. In some aspects, the cells of the cell suspension are passed through an electric field generated by at least one electrode. In some aspects, the electric field is between about 0.1 kV/m to about 100 MV/m, or any number or range of numbers therebetween. In some aspects, an integrated circuit is used to provide an electrical signal to drive the electrodes. In some aspects, the cells of the cell suspension are exposed to the electric field for a pulse width of between about 1 ns to about 1 s, a period of between about 100 ns to about 10 s, or any time or range of times therebetween.

In some aspects, cell deformation devices 204 can comprise one or more constrictions, wherein the one or more constrictions are pores or contained within pores. In some aspects, the pore is contained in a surface. In some aspects, the surface is a filter. In some aspects, the surface is a membrane. In some aspects, the constriction size is a function of the cell diameter. In some aspects, the constriction size is about 20%, about 30%, about 40%, about 50%, about 60%, about 70%, about 80%, about 90%, or about 99% of the cell diameter. Examples of cell deformation devices comprising pores for use with the systems, methods, and kits described herein are described in PCT/US2016/050287, which is hereby incorporated by reference in its entirety.

In some aspects, the cell deformation subsystem components of FIG. 14 can be releasably coupled to a frame of a POC system, such as presented in FIG. 6-FIG. 10.

In some aspects, the cell-deformation subsystem further comprises one or more of the following components: a rigid sample vessel, a cell-aggregate filter, a preparation vessel, a rigid reservoir assembly, one or more microfluidic chip cartridges, one or more microfluidic chips, tubing, a tube fitting, a connector, a clamp, an air filter, and a barrel filter.

### 3. Dilution Subsystem (Zone 3)

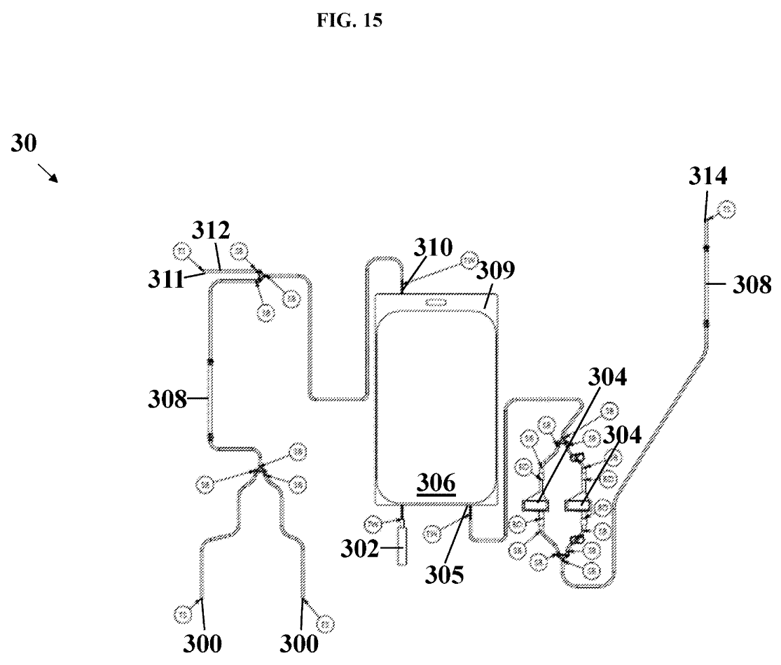

In some aspects, a system for processing cells as described herein comprises a dilution subsystem. In some aspects, cell suspension comprising engineered cells flows from the cell deformation subsystem to the dilution subsystem, where the cell suspension comprising engineered cells can rest in a container of the dilution subsystem. In some aspects, the dilution subsystem can be used to dilute the cell suspension comprising engineered cells in a desired buffer, such as to achieve a desired concentration of cells or a desired buffer condition prior to incubation. In some instances, following cell deformation, a dilution step may not be needed, and, as such, the dilution subsystem is not included in the system. In some aspects, the dilution subsystem comprises dilution subsystem 30 presented in FIG. 15. In some aspects, dilution subsystem 30 comprises cell suspension inlet 311, which inlet is fluidically connected to the cell deformation subsystem via tubing 312. In some aspects, the cell suspension inlet 311 is further fluidically connected to container 306, such as a bag, wherein the connection to the container can be via outlet 310. In some aspects, container 306 can contact plate 309. Plate 309 is in some instances temperature-controlled, such as heated, cooled, or kept at a relatively constant temperature. In some aspects, container 306 is further connected to sample bulb 302. In some aspects, fluid, such as sample, can flow into sample bulb 302 from container 306. In some instances, the sample bulb can be welded off, and the fluid in the sample bulb can be analyzed. In some aspects, container 306 is fluidically connected via inlet 310 and tubing 312 to pump tubing subassembly 308 and to waste outlet 300. Container 306 can be further fluidically connected via outlet 305 and tubing 312 to aggregate filters 304. Aggregate filters 304 can be connected via tubing 312 to pump tubing subassembly 308 and further fluidically connected to an incubation subsystem via outlet 314, such as incubation subsystem 40. Further referring to FIG. 15, TW represents tube weld; SB represents solvent bond; and TS represents seal.

In some aspects, the dilution subsystem components of FIG. 15 can be releasably coupled to a frame of a POC system, such as presented in FIG. 6-FIG. 10. In some aspects as discussed above, the dilution subsystem components can be omitted from the point of care system in instances where dilution is not needed. In some aspects, the dilution subsystem can also act as the incubation subsystem (Zone 4), provided the container 306 is in contact with a temperature-controlled plate.

In some aspects, the dilution subsystem comprises a scale system for weighing the container. In some aspects, the scale system for weighing the container comprises a tension load cell. In some aspects, the scale system for weighing the container comprises a compression load cell. In some aspects, the scale system for weighing the container comprises a straight bar load cell. In some aspects, the dilution subsystem comprises a scale system for measuring an amount of buffer added to the cells of the cell suspension comprising engineered cells. In some aspects, the scale system for measuring the amount of buffer comprises a tension load cell. In some aspects, the scale system for measuring the amount of buffer comprises a compression load cell. In some aspects, the scale system for measuring the amount of buffer comprises a straight bar load cell. In some aspects, the dilution subsystem comprises at least one outlet configured to be coupled to the container configured to receive the cell suspension comprising engineered cells. In some aspects, the container is a bag comprising at least one inlet and at least one outlet. In some aspects, the container is a bag such as bag 80 of FIG. 20.

In some aspects, the dilution subsystem further comprises an agitation system comprising a platform in contact with the container configured to rock the container up and down. In some aspects, the first agitation system comprising a platform in contact with the container is configured to rock the container up and down to agitate the cell suspension to promote homogeneity of the cell suspension comprising engineered cells or the diluted cell suspension. In some aspects, the dilution subsystem further comprises a first agitation system comprising a plate configured to contact a container and to move in and out while contacting the container, such as the agitation system presented in FIG. 25. In some aspects, the agitation system is part of the frame of the system.

In some aspects, the dilution subsystem further comprises an illumination system configured to illuminate the cell suspension comprising engineered cells and the diluted cell suspension. Such an illumination system can allow an operator to visually inspect a container for undesirable materials, such as cellular aggregates or debris. In some aspects, the dilution subsystem further comprises a second agitation system comprising a platform in contact with the container configured to rock the container up and down. In some aspects, the second agitation system is configured to rock the container up and down to agitate the cell suspension comprising engineered cells or the diluted cell suspension to promote homogeneity of the cell suspension comprising engineered cells or the diluted cell suspension. In some aspects, the dilution subsystem further comprises a second agitation system comprising a plate configured to contact the container and to move in and out while contacting the container, such as the agitation system presented in FIG. 25. In some aspects, the agitation system is part of the frame of the system. In some aspects, the dilution subsystem comprises at least one pump configured to move fluid between fluidically connected subsystems.

In some aspects, the dilution subsystem further comprises one or more of the following components: a cell aggregate filter, tubing, a tube fitting, a connector, a clamp, a sampling bulb, and a carboy.

### 4. Incubation Subsystem (Zone 4)

In some aspects, a system for processing cells as described herein comprises an incubation subsystem. In some aspects, diluted cell suspension flows from the dilution subsystem to the incubation subsystem, where the diluted cell suspension can be incubated in a container of the incubation subsystem. In some aspects, the temperature of incubation ranges from about 10° C. to about 40° C., about 15° C. to about 40° C., about 20° C. to about 40° C., about 25° C. to about 40° C., or about 30° C. to 40° C. In some instances, following cell deformation, a dilution step may not be needed, and, as such, the dilution subsystem is not included in the system and the incubation subsystem is fluidically connected to the cell deformation subsystem. In some instances, the incubation subsystem can be used to perform dilution of a cell suspension comprising engineered cells to form a diluted cell suspension. In some aspects, the incubation subsystem can comprise one or more additional inlets, such as inlets for delivering buffer to Zone 4, to allow Zone 4 to dilute a cell suspension comprising engineered cells.

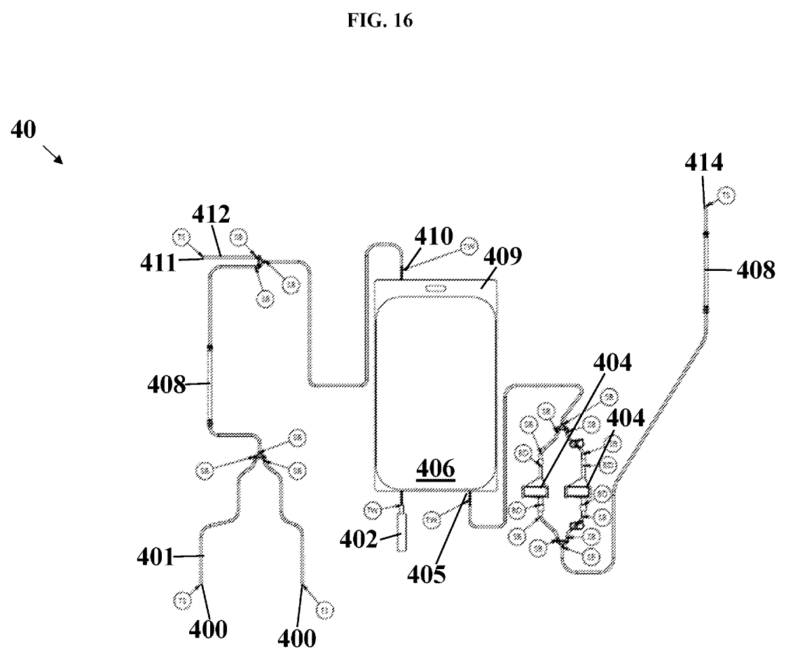

In some aspects, the incubation subsystem comprises dilution subsystem 40 presented in FIG. 16. In some aspects, incubation subsystem 40 comprises diluted cell suspension inlet 411, which inlet is fluidically connected to the dilution subsystem via tubing 412. In some aspects, the cell suspension inlet 411 is further fluidically connected to container 406, such as a bag, wherein the connection to the container can be via outlet 410. In some aspects, container 406 can contact plate 409. Plate 409 is in some instances temperature-controlled, such as heated, cooled, or kept at a relatively constant temperature. In some aspects, container 406 is further fluidically connected to sample bulb 402. In some aspects, fluid, such as sample, can flow into sample bulb 402 from container 406. In some instances, the sample bulb can be welded off, and the fluid in the sample bulb can be analyzed. In some aspects, container 406 is fluidically connected via inlet 410 and tubing 412 to pump tubing subassembly 408 and to waste outlet 400. Container 406 can be further fluidically connected via outlet 405 and tubing 412 to aggregate filters 404. Aggregate filters 404 can be connected via tubing 412 to pump tubing subassembly 408 and further fluidically connected to a cell washing subsystem via outlet 414, such as cell washing subsystem 50. Further referring to FIG. 16, TW represents tube weld; SB represents solvent bond; and TS represents seal.

In some aspects, the incubation subsystem components of FIG. 16 can be releasably coupled to a frame of a POC system, such as presented in FIG. 6-FIG. 10. In some aspects as discussed above, the dilution subsystem components can be omitted from the point of care system in instances where dilution is not needed, and, as such, Zone 2 is fluidically connected to Zone 4. In some aspects, the incubation subsystem can also act as the dilution subsystem (Zone 3), provided the incubation subsystem comprises one or more inlets for introducing a fluid, such as a buffer, with which to dilute the cell suspension comprising engineered cells.

In some aspects, the incubation subsystem comprises a temperature control device configured to adjust a temperature of the diluted cell suspension. In some aspects, the temperature control device is a heated plate. In some aspects, the heated plate is heated plate 70 as presented in FIG. 19. In some aspects, the plate is a part of the frame of the system. In some aspects, the incubation subsystem comprises a scale system configured to measure an amount of the buffer added to the cells of the diluted cell suspension. In some aspects, the scale system comprises a tension load cell. In some aspects, the scale system comprises a compression load cell. In some aspects, the scale system comprises a straight bar load cell. In some aspects, the incubation subsystem comprises a first agitation system comprising a platform in contact with the container configured to rock the container up and down. In some aspects, wherein the first agitation system comprising a platform in contact with the container is configured to rock the container up and down to agitate the diluted cell suspension to promote homogeneity of the diluted cell suspension or the incubated cell suspension. In some aspects, the incubation subsystem comprises a second agitation system comprising a platform in contact with the container configured to rock the container up and down. In some aspects, the second agitation system comprising a platform in contact with the container is configured to rock the container up and down to agitate the cell suspension to promote homogeneity of the diluted cell suspension or the incubated cell suspension. In some aspects, the first and second agitation systems are a part of the frame of the system. In some aspects, the incubation subsystem comprises at least one pump configured to move fluid between fluidically connected subsystems. In some aspects, the incubation subsystem further comprises one or more of the following components: a cell aggregate filter, tubing, a tube fitting, a connector, and a clamp.

### 5. Cell Washing Subsystem

In some aspects, a system for processing cells as described herein comprises a cell washing subsystem. In some aspects, incubated cell suspension flows from the incubation subsystem to the cell washing subsystem, where the incubated cell suspension can be washed, such as buffer exchanged, into a desired buffer, such as a cryoprotectant media. Buffer exchange can occur using one or more cell suspension devices of Zone 5, such as TFF filter assemblies. In some aspects, the cell washing subsystem can be omitted from the point of care system, and instead the components of Zone 1 can be used to perform the cell washing performed by the cell washing subsystem (Zone 5).

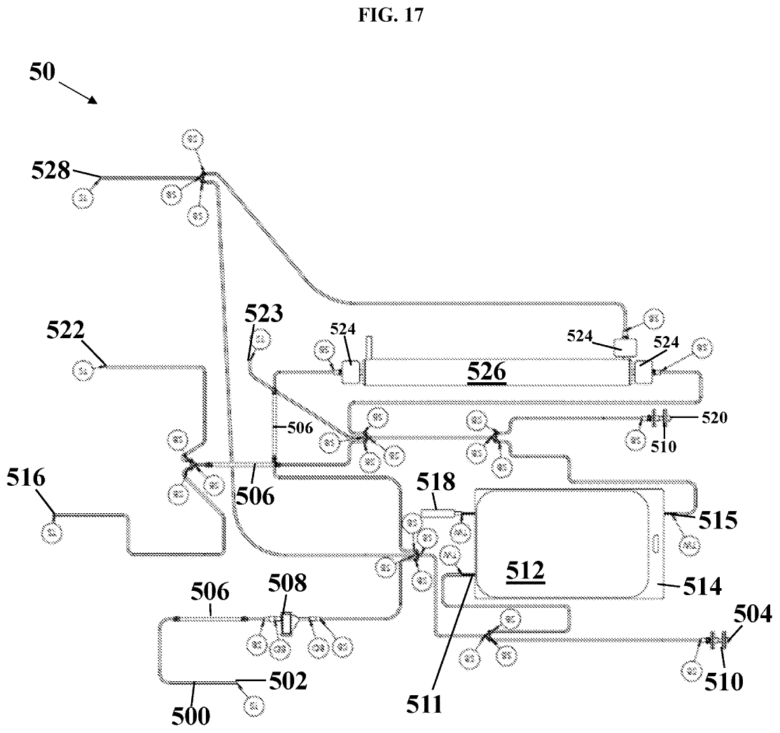

In some aspects, the cell washing subsystem comprises cell washing subsystem 50 presented in FIG. 17. In some aspects, cell washing subsystem 50 comprises incubated cell suspension inlet 528, which is fluidically connected to an incubation subsystem, such as incubation subsystem 40, via tubing 500. In some aspects, incubated cell suspension inlet 528 is further fluidically connected to a cell suspension device 526, such as a tangential flow filtration (TFF) filter assembly, via tubing 500. Cell suspension device 526, such as a TFF filter assembly, can comprise pressure sensors 524 connected to the inlets and outlet of the cell suspension device 526. In some aspects, cell suspension device 526 is further fluidically connected to container 512 via tubing 500 and pump tubing subassembly 506. In some aspects, container 512 is a bag. In some aspects, container 512 contacts plate 514, which in some instances is a temperature-controlled plate, such as heated, cooled, or kept at a relatively constant temperature. In some aspects, container 512 is fluidically connected to sample bulb 518. In some aspects, fluid, such as sample, can flow into sample bulb 518 from container 512. In some instances, the sample bulb can be welded off, and the fluid in the sample bulb can be analyzed. In some aspects, container 512 comprises inlet 515 and outlet 511, which inlet and outlet are coupled to tubing. In some aspects, cell washing subsystem comprises buffer inlet 520 which is coupled to filters 510, which can be 0.2 μm air filters. In some aspects, cell washing subsystem comprises buffer inlet 523 which is fluidically connected to container 512 by tubing 500. In some aspects, cell washing subsystem 50 comprises preservation media inlet 504, which is coupled to filters 510, which can be 0.2 μm air filters, and further is fluidically connected to container 512 via tubing 500. In some aspects, the cell washing subsystem comprises waste outlets 522 and 516. In some aspects, container 512 is fluidically connected via tubing 500 to aggregate filter 508, which can be which can be a 40 μm aggregate filter. In some aspects, aggregate filter 508 is fluidically connected via tubing 500 and pump tubing subassembly 506 to outlet 502, which outlet 502 is fluidically connected to a container filling subsystem, such as container filling subsystem 60. Further referring to FIG. 17, TW represents tube weld; SB represents solvent bond; and TS represents seal.

In some aspects, the cell washing subsystem components of FIG. 17 can be releasably coupled to a frame of a POC system, such as presented in FIG. 6-FIG. 10. In some aspects, the cell washing subsystem can be omitted from the point of care system, and the components of the suspension preparation subsystem (Zone 1) can be used to perform cell washing, such as exchanging an incubated cell suspension into a preservation media, e.g., a cryopreservation media.

In some aspects, the cell-washing subsystem comprises a device configured to perform a buffer exchange operation. In some aspects, the device comprises a tangential flow filtration system configured to perform the buffer exchange operation. In some aspects, the cell-washing subsystem comprises a scale system configured to measure an amount of the buffer added to the cells during the buffer exchange operation. In some aspects, the scale system is a tension load cell. In some aspects, the scale system is a compression load cell. In some aspects, the scale system is a straight bar load cell. In some aspects, the cell-washing subsystem comprises at least one outlet configured to be coupled to the container. In some aspects, the container is a bag comprising at least one inlet and at least one outlet. In some aspects, the cell-washing subsystem comprises an agitation system comprising a platform in contact with the container configured to rock the container up and down. In some aspects, the agitation system comprising a platform in contact with the container is configured to rock the container up and down to agitate the incubated cell suspension or the cells suspended in preservation media to promote homogeneity of the diluted cell suspension or the cells suspended in preservation media. In some aspects, the cell-washing subsystem comprises an illumination system configured to illuminate the diluted cell suspension or the cells suspended in the preservation media. Such an illumination system can allow an operator to visually inspect a container for undesirable materials, such as cellular aggregates or debris. In some aspects, the cell-washing subsystem further comprises one or more of the following components: a cell aggregate filter, tubing, a tube fitting, a connector, a clamp, a sampling bulb, an air filter, and a carboy

In some aspects, the preservation media is a cryoprotectant media. The cryoprotectant media can be any cryoprotectant media known in the art, such as, but not limited to, DMSO, glycerol, ethylene glycol, propylene glycol, sucrose, trehalose, 2-Methyl-2,4-pentanediol (MPD), sorbitol, proline, glycerol 3-phosphate, and formamide.

### 6. Container Filling Subsystem

In some aspects, a system for processing cells as described herein comprises a container filling subsystem. In some aspects, the container filling subsystem can be used to remove aggregates and debris from the cells suspended in preservation media by using one or more aggregate filters of the container filling subsystem, and subsequently filling one or more containers of the subsystem with the processed cells. In some aspects, the container filling subsystem can comprise a scale system to ensure that each of the one or more containers comprising the processed cells are filled to a desired volume.

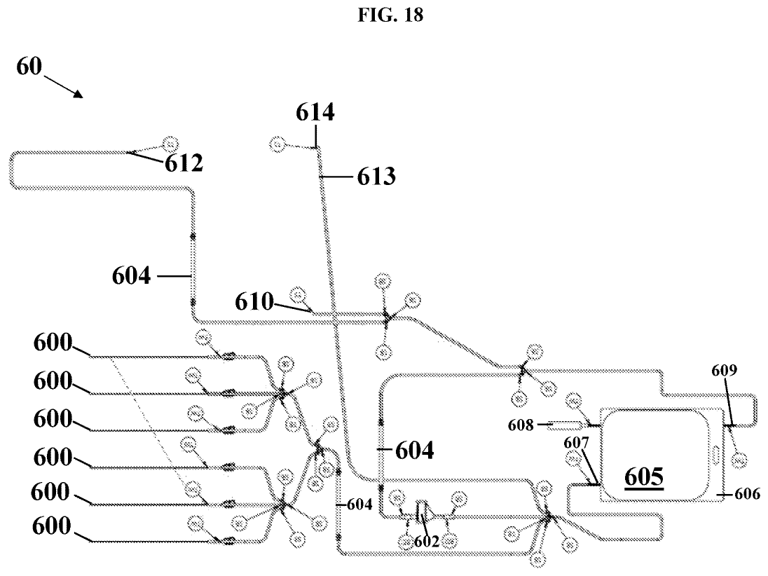

In some aspects, the system for processing cells comprises container filling subsystem 60 presented in FIG. 18. In some aspects, the container filling subsystem 60 comprises an inlet for receiving cells suspended in preservation media 614, which inlet is fluidically connected via tubing 613 to container 605. In some instances, container 605 can be a bag. In some instances, container 605 contacts plate 606, which plate can be temperature-controlled, such as heated, cooled, or kept at a relatively constant temperature. In some aspects, container 605 is fluidically connected to sample bulb 608. In some aspects, fluid, such as sample, can flow into sample bulb 608 from container 406. In some instances, the sample bulb can be welded off, and the fluid in the sample bulb can be analyzed. In some aspects, container 605 comprises inlet 609 which is coupled to tubing. In some aspects, container 605 is further fluidically connected via outlet 607, tubing 613, and pump tubing subassembly 604 to outlets 600, which outlets are configured to be coupled to containers for received processed cells. In some aspects, container filling subsystem 60 further comprises waste outlet 612. In some aspects, container filling subsystem 60 further comprises buffer inlet 610. In some aspects, container filling subsystem 60 further comprises aggregate filter 602 which is fluidically connected to container 605 via tubing 613. Further referring to FIG. 18, TW represents tube weld; SB represents solvent bond; and TS represents seal.

In some aspects, the container filling subsystem components of FIG. 18 can be releasably coupled to a frame of a POC system, such as presented in FIG. 6-FIG. 10. In some aspects, the number of containers for receiving process cells can be 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 individual containers, such as bags or vials. In some aspects, the number of containers for receiving process cells can be 5 to 20 containers.

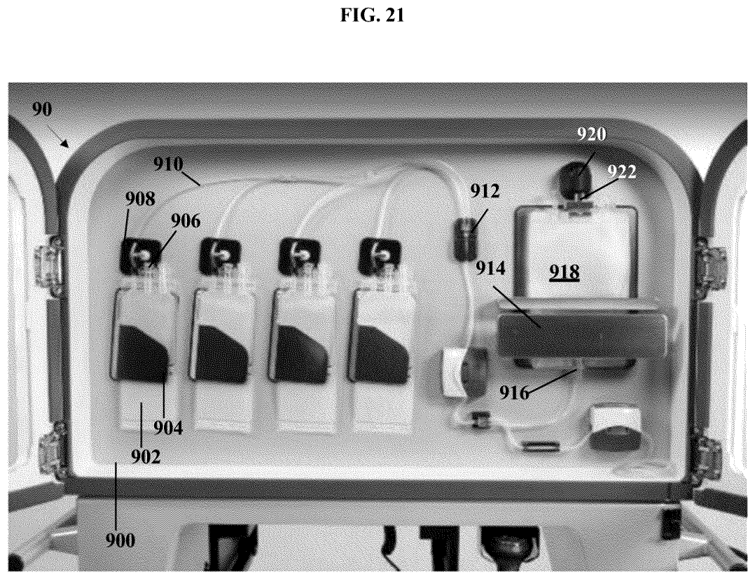

In some aspects, container filling subsystem 60 further comprises a container filling station, such as container filling station 90 presented in FIG. 21. In some aspects, container filling station 90 comprises housing 900. In some aspects, the filling station further comprises containers 904 comprising inlets 906, which containers are in contact with scale systems 902. Container 904 can further be coupled to valve 908. Scale systems 902 can comprise, for example, compression load cells, tension load cells, or straight bar load cells. In some aspects, the container filling station comprises container 918, which comprises inlet 922 coupled to valve 920. Container 918 also comprises outlet 916 which is coupled to tubing 910. Moreover, container 918 is in contact with agitation system 914. Container filling station 90 can further comprise valves, such as 912, to control fluid flow in the station.

In some aspects, the container-filling subsystem comprises a scale system configured to measure an amount of the cells suspended in the preservation media added to the one or more containers. In some aspects, the scale system comprises a tension load cell. In some aspects, the scale system comprises a compression load cell. In some aspects, the scale system comprises a straight bar load cell. In some aspects, the container-filling subsystem comprises an agitation system comprising a platform in contact with the container configured to rock the container up and down. In some aspects, the agitation system comprising a platform in contact with the container is configured to rock the container up and down to agitate the cells suspended in preservation media to promote homogeneity of the cells suspended in the preservation media in the one or more containers. In some aspects, the container-filling subsystem comprises an illumination system configured to illuminate the cells suspended in the preservation media in the one or more containers. In some aspects, the container-filling subsystem comprises one or more outlets configured to be coupled to the one or more containers. In some aspects, the one or more containers of the cell-washing subsystem comprises one or more bags comprising at least one inlet and at least one outlet. In some aspects, the container-filling subsystem comprises at least one pump configured to move fluid within the subsystem or between fluidically connected subsystems. In some aspects, the container-filling subsystem further comprises one or more of the following components: a cell aggregate filter, tubing, a tube fitting, a connector, a clamp, and a sampling bulb.

### 7. Temperature Control Subsystem

In some aspects, a system for processing cells as described herein comprises a temperature control subsystem. In some aspects, the temperature control subsystem can be used to perform the functions otherwise performed by the dilution subsystem (Zone 3) and incubation subsystem (Zone 4). For instance, in some aspects, cell suspension comprising engineered cells flows from the cell deformation subsystem to the temperature control subsystem, where the cell suspension comprising engineered cells can rest in a container of the temperature control subsystem. In some aspects, the temperature control subsystem can be used to dilute the cell suspension comprising engineered cells in a desired fluid or buffer and/or to add a dry reagent to the cell suspension, such as to achieve a desired concentration of cells or a desired buffer condition prior to incubation. In some aspects, the diluted cell suspension can be incubated in a container of the temperature control subsystem. In some aspects, the temperature of incubation ranges from about 10° C. to about 40° C., about 15° C. to about 40° C., about 20° C. to about 40° C., about 25° C. to about 40° C., or about 30° C. to 40° C. In some aspects, the container is further fluidically connected to a sample bulb. In some aspects, fluid, such as sample, can flow into sample bulb from the container. In some instances, the sample bulb can be welded off, and the fluid in the sample bulb can be analyzed. In some aspects, the temperature control subsystem comprises a temperature control device configured to adjust a temperature of the diluted cell suspension. In some aspects, the temperature control device is a heated plate. In some aspects, the heated plate is heated plate 70 as presented in FIG. 19. In some aspects, the plate is a part of the frame of the system.

In some aspects, the temperature control subsystem components can be releasably coupled to a frame of a POC system, such as presented in FIG. 6-FIG. 10.

In some aspects, the temperature control subsystem comprises a scale system for weighing the container. In some aspects, the scale system for weighing the container comprises a tension load cell. In some aspects, the scale system for weighing the container comprises a compression load cell. In some aspects, the scale system for weighing the container comprises a straight bar load cell. In some aspects, the temperature control subsystem comprises a scale system for measuring an amount of buffer added to the cells of the cell suspension comprising engineered cells. In some aspects, the scale system for measuring the amount of buffer comprises a tension load cell. In some aspects, the scale system for measuring the amount of buffer comprises a compression load cell. In some aspects, the scale system for measuring the amount of buffer comprises a straight bar load cell. In some aspects, the temperature control subsystem comprises at least one outlet configured to be coupled to the container configured to receive the cell suspension comprising engineered cells. In some aspects, the container is a bag comprising at least one inlet and at least one outlet. In some aspects, the container is a bag such as bag 80 of FIG. 20.

In some aspects, the temperature control subsystem further comprises an agitation system comprising a platform in contact with the container configured to rock the container up and down. In some aspects, the first agitation system comprising a platform in contact with the container is configured to rock the container up and down to agitate the cell suspension to promote homogeneity of the cell suspension comprising engineered cells or the diluted cell suspension. In some aspects, the dilution subsystem further comprises a first agitation system comprising a plate configured to contact a container and to move in and out while contacting the container, such as the agitation system presented in FIG. 25. In some aspects, the agitation system is part of the frame of the system.

In some aspects, the temperature control subsystem further comprises an illumination system configured to illuminate the cell suspension comprising engineered cells and the diluted cell suspension. Such an illumination system can allow an operator to visually inspect a container for undesirable materials, such as cellular aggregates or debris. In some aspects, the dilution subsystem further comprises a second agitation system comprising a platform in contact with the container configured to rock the container up and down. In some aspects, the second agitation system is configured to rock the container up and down to agitate the cell suspension comprising engineered cells or the diluted cell suspension to promote homogeneity of the cell suspension comprising engineered cells or the diluted cell suspension. In some aspects, the dilution subsystem further comprises a second agitation system comprising a plate configured to contact the container and to move in and out while contacting the container, such as the agitation system presented in FIG. 25. In some aspects, the agitation system is part of the frame of the system. In some aspects, the dilution subsystem comprises at least one pump configured to move fluid between fluidically connected subsystems.

In some aspects, the temperature control subsystem further comprises one or more of the following components: a cell aggregate filter, tubing, a tube fitting, a connector, a clamp, a sampling bulb, and a carboy.

ii. Reservoirs

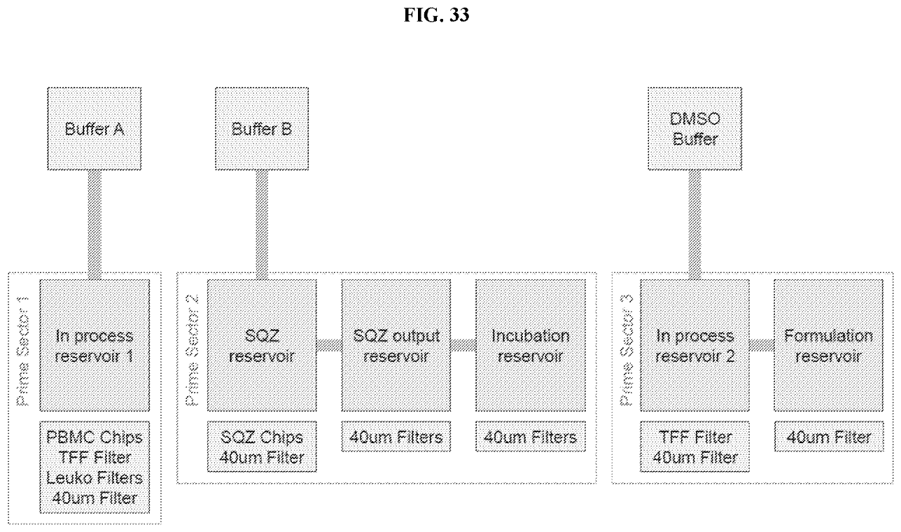

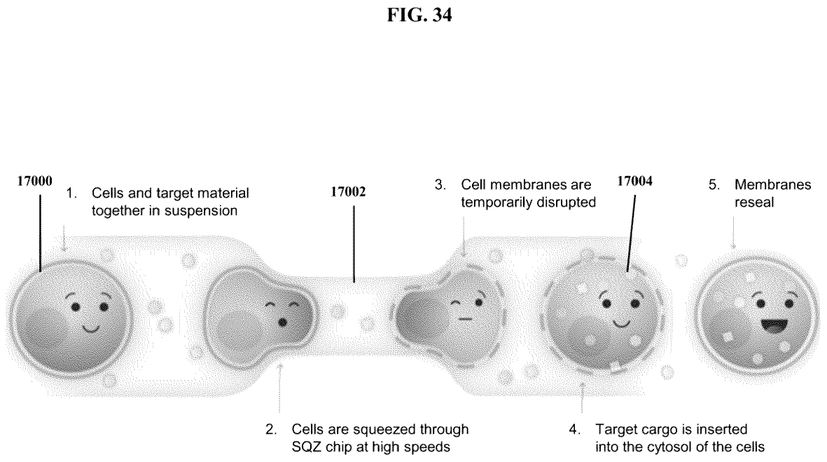

In some aspects, a system for processing cells as described herein comprises one or more containers in each zone of the system. In some instances, these containers can be referred to as “reservoirs,” which reservoirs (or containers) can have various different volumes, such as presented in FIG. 33 and FIG. 34.

Referring to FIG. 33, in some instances, Zone 1, represented as in process reservoir 1 of FIG. 33 can be primed with a desired buffer, such as Buffer A of FIG. 33, which can be any desired buffer. As such, the components of Zone 1, such as cell isolation devices, TFF filter assembly, aggregate filters, etc., are primed with the desired buffer. In some instances, Zones 2, 3, and 4, represented as SQZ reservoir, SQZ output reservoir, and incubation reservoir of FIG. 33, can be primed with a desired buffer, such as Buffer B of FIG. 33, which can be any desired buffer. As such, the components of Zones 2-4, such as the cell deformation devices, aggregate filters, etc., can be primed with the desired buffer. In some instances, Buffer B is only connected to Zone 2, but Zone 2 is in fluid communication with Zone 3, and Zone 3 in communication with Zone 4, thereby allowing Buffer B to prime each of Zones 2-4. In some aspects, Zones 5 and 6, represented as in process reservoir 2 and formulation reservoir of FIG. 33, can be primed with a desired buffer, such as DMSO buffer. As such, the components of Zones 5 and 6, such as TFF filter assembly, aggregate filters, etc., can be primed with the desired buffer. In some instances, the buffer, e.g., DMSO, is in fluid communication with Zone 5, and Zone 5 is in fluid communication with Zone 6, thereby allowing the desired buffer to prime both Zone 5 and Zone 6.

In some aspects, the volume of the reservoir (container) can be about 0.10 L to about 4.00 L, about 0.20 L to about 4.00 L, about 0.30 L to about 4.00 L, about 0.40 L to about 4.00 L, about 0.50 L to about 4.00 L, about 0.75 L to about 4.00 L, about 1.00 L to about 4.00 L, about 1.25 L to about 4.00 L, about 1.50 L to about 4.00 L, about 1.75 L to about 4.00 L, about 2.0 L, to about 4.00 L, about 2.25 L, to about 4.00 L, about 2.50 L, to about 4.00 L, about 2.75 L to about 4.00 L, about 3.00 L to about 4.00 L, about 3.25 L to about 4.00 L, about 3.50 L to about 4.00 L, about 3.75 L to about 4.00 L, or about 4.00 L.

In some aspects, reservoirs of the system can be fluidically connected to a supply of buffer. In some aspects, at least one buffer line can be connected to at least one reservoir. In some aspects, at least one first reservoir is connected to at least one second different reservoir. In some aspects, buffer can be transported from a first reservoir to a second reservoir. In some aspects, the system for processing cells comprises at least one, at least two, or at least three prime sectors, as presented in FIG. 33.

iii. Pressure Testing

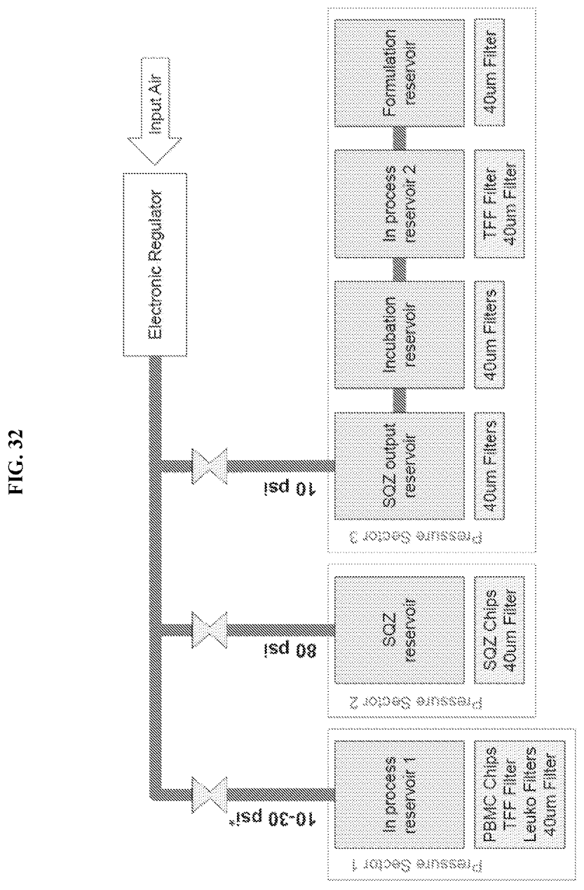

In some aspects, a system for cell processing as described herein can undergo pressure testing of one or more subsystems. For instance, pressure testing can occur as presented in the schematic of FIG. 32. Referring to FIG. 32, the suspension preparation subsystem can be tested a pressure range of 10-30 psi, and components such as the cell suspension device, cell isolation device, leukoreduction filters, and aggregate filters can be subject to the pressure testing. In some aspects, the cell deformation subsystem can be tested at about 80 psi, and components such as the aggregate filters and cell deformation device can be subjected to the pressure test. In some aspects, the dilution subsystem, incubation subsystem, cell washing subsystem, and container filling subsystem can be connected to the same line for pressure testing, as presented in FIG. 32, and the pressure test can be conducted at about 10 psi. Components such as aggregate filters and TFF filter assemblies can be subjected to the pressure integrity test. In some aspects, the pressure testing can comprise use of an electronic regulator, which regulates air pressure in the system. In some aspects, the electronic regulator regulates the pressure for each of the pressure sectors tested. In some aspects, valves regulate the flow of air into each of the pressure sectors tested, such as depicted by the bowtie shapes of FIG. 32. In some aspects, the pressure testing can be used to test the integrity of each disposable kit following coupling of the components of the kit to the frame of the system. In some aspects, each of the cells suspension subsystem (Zone 1), the cell deformation subsystem (Zone 2), the dilution subsystem (Zone 3), and the container filling subsystem (Zone 6) are individually pressure tested, and the incubation subsystem (Zone 4) and cell washing subsystem (Zone 5) are pressure tested together.

c. Cells for Processing

In some aspects, the system for processing cells processes red blood cells (RBC). In some aspects, the system for processing cells processes peripheral blood mononuclear cells (PBMCs). In some aspects, the system for processing cells processes activating antigen carrier (AAC) cells. In some aspects, the system for processing cells processes tolerizing antigen carrier (TAC) cells. In some aspects, the system for processing cells process antigen presenting cells (APCs). In some aspects, the system for processing cells processes T cells. In some aspects, the system for processing cells processes B cells. In some aspects, the system for processing cells processes macrophages. In some aspects, the system for processing cells processes natural killer (NK) cells. In some aspects, the system for processing cells processes dendritic cells. In some aspects, the system for processing cells processes immune cells. In some aspects, the system for processing cells processes monocytes. In some aspects, the system for processing cells processes monocytes leukocytes. In some aspects, the system for processing cells processes eosinophils. In some aspects, the system for processing cells processed basophils. In some aspects, the system for processing cells processes natural killer T (NKT) cells. In some aspects, the system for processing cells processes mast cells. In some aspects, the system for processing cells processes neutrophils. In some aspects, the cell deformation subsystem comprises one or more cell-deforming constrictions configured to cause perturbations in cell membranes of the cells to allow entry of a payload into the cell. In some aspects, the payload comprises one or more reprogramming factors. In some aspects, the payload comprises one or more nucleic acids. In some aspects, the payload comprises one or more differentiation factors. In some aspects, the payload comprises one or more neuron reprogramming factors. In some aspects, the system processes cells for cell therapeutics, e.g., cell-based therapeutics. In some aspects, the cells for processing can comprise cells of an enriched leukapheresis product such as a LEUKOPAK, or a similar product.

d. Process Flows

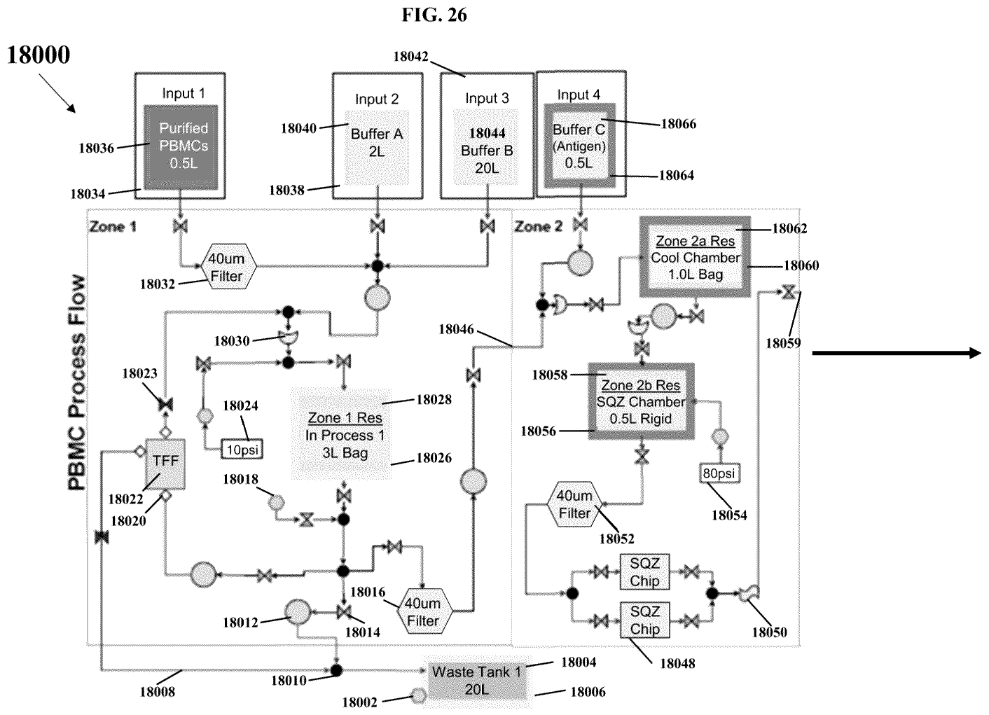

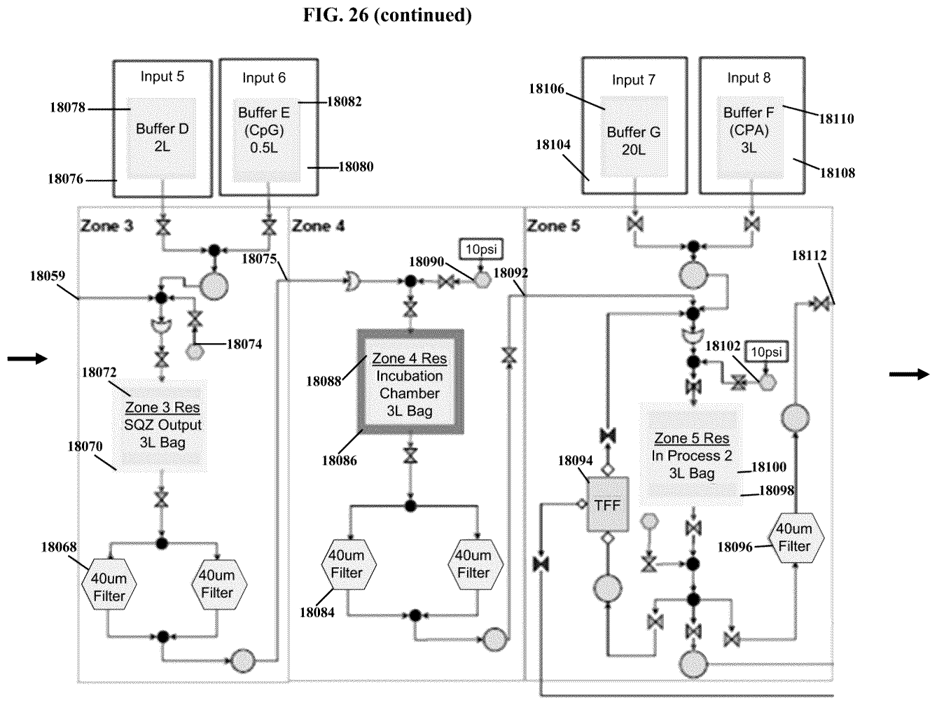

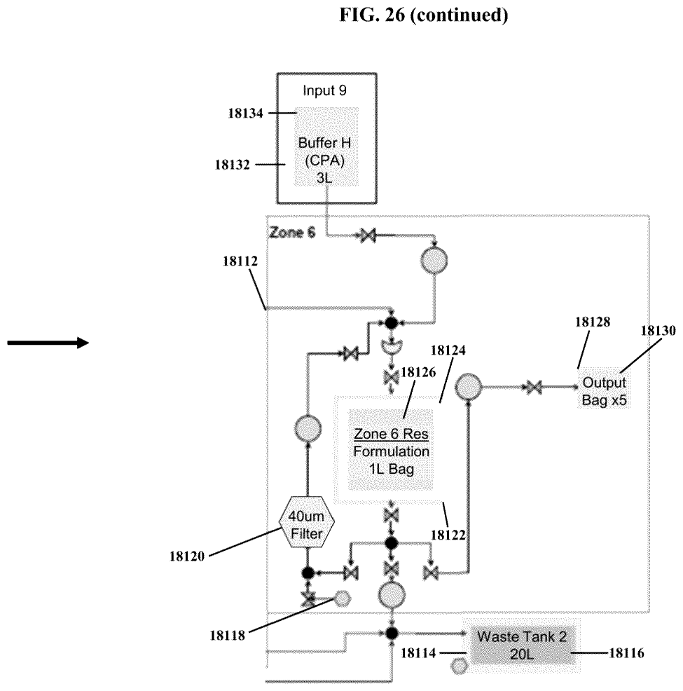

As discussed above, a point of care system for processing cells as described herein can comprise one or more subsystems, i.e., zones, for processing cells. Each of the subsystems can be fluidically connected to a second subsystem, thereby allowing exchange of fluids between the zones. In some aspects, a system for processing cells as described herein comprises a suspension preparation subsystem (Zone 1), a cell deformation subsystem (Zone 2), a dilution subsystem (Zone 3), an incubation subsystem (Zone 4), a cell washing subsystem (Zone 5), and a container filling subsystem (Zone 6). In some aspects, process flows for the systems described herein are presented in FIG. 26-FIG. 31, as further described infra.

The process flows for RBC and PBMC can in some instances be the same, however, some aspects can be different. For instance, in some aspects, the process flow for RBC and PBMC is different in the suspension preparation subsystem (Zone 1) and the cell deformation subsystem. In some aspects, the suspension preparation subsystem process flow for RBC and PBMC is the same in the dilution subsystem (Zone 3), the incubation subsystem (Zone 4), the cell washing subsystem (Zone 5), and the container filling subsystem (Zone 6).

Referring now to FIG. 26, in some aspects, a point of care system process flow is a flow as presented in 18000. It is noted that solid black lines 18008 represent flow paths. Grey hexagons 18002 represent filters, which in some aspects are air filters. Black circles 18010 represent tubing junctions. Grey circles 18012 represent pumps. Black bowties 18023 represent valves, which in some aspects can be proportional pinch valves. Grey bowties 18014 represent valves, which can be in some aspects pinch valves. White diamonds 18020 represent pressure gauges. Grey crescents 18030 represent bubble sensors. Wavy rectangles 18050 represent flow sensors. In some aspects, containers such as 18036, 18040, and 18044 can be fluidically connected to Zone 1 via tubing. In some aspects, container 18036 can be in contact with plate 18034, which can be a temperature-controlled plate. Container 18036 can further be fluidically connected to filter 18032, which can be an 40 μm aggregate filter, and to container 18028 via tubing. In some aspects, container 18040 contacts plate 18038, which can be a temperature-controlled plate. Container 18040 can be fluidically connected to container 18028 via tubing. In some aspects, container 18044 contacts plate 18042, which can be a temperature-controlled plate. Container 18044 can further be in fluidic communication with container 18028 via tubing. Container 18028 can contact plate 18026, which in some aspects can be a temperature controlled plate. In some aspects, container 18028 can contact a scale system, such as a compression load cell or a tension load cell. In some aspects, container 18028 can be in fluidic communication with cell suspension device 18022, which can be a TFF filter assembly, via tubing. In some aspects, cell suspension device 18022 is fluidic communication with pressure gauges, such as pressure gauge 18020. In some aspects, inlet 18024 can be used as an inlet for pressure testing Zone 1 of the point of care system. In some aspects, waste from Zone 1 can flow via tubing into waste container 18004, which can be in contact with scale system 18006, which can be a tension load cell or a compression load cell in some instances. In some aspects, container 18028 is in fluidic communication with filter 18016, which can be a 40 μm aggregate filter, and further in fluidic communication with Zone 2 via tubing and Zone 1 outlet 18046.

Further referring to FIG. 26, Zone 1 can be in fluidic communication with Zone 2 via Zone 1 outlet 18046, which serves as an inlet for Zone 2 such that fluid, e.g., cells suspended in delivery media, can flow from Zone 1 to Zone 2. Zone 1 outlet 18046 can be in fluidic communication with container 18062 via tubing. In some aspects, container 18062 can contact plate 18060, such as a temperature-controlled plate for heating, cooling, or maintaining a relatively constant temperature. In some aspects, container 18066 is in contact with plate 18064, which can be a temperature controlled plate. In some aspects, container 18066 can be in fluidic communication with container 18062 via tubing. In some aspects, container 18062 is in fluidic communication with container 18058 via tubing. In some aspects, container 18058 contacts plate 18056, such as a temperature-controlled plate for heating, cooling, or maintaining a relatively constant temperature. In some aspects, container 18058 is in fluidic communication with filter 18052, such as a 40 μm aggregate filter, and is further in fluidic communication with cell deformation devices 18048 via tubing. In some aspects, inlet 18054 is used for pressure testing of Zone 2. In some aspects, cell deformation devices 18048 are in fluidic communication with Zone 3 via Zone 2 outlet 18059, which can also be the inlet of Zone 3.

Further referring to FIG. 26, in some aspects, Zone 3 inlet 18059 can be in fluidic communication with container 18072 via tubing. Container 18072 can contact scale system 18070 which can be a tension load cell or a compression load cell for instance. Container 18078 can contact plate 18076, which can be a temperature-controlled plate. Container 18078 can be in fluidic communication with container 18072 via tubing. In some aspects, container 18082 contact plate 18080, which can be a temperature-controlled plate. Container 18082 can be in fluidic communication with container 18072 via tubing. In some aspects, inlet 18074 is in fluidic communication with container 18072. IN some aspects, container 18072 is in fluidic communication with aggregate filter 18068, which can be a 40 μm aggregate filter, and further in fluidic communication with Zone 3 outlet 18075 via tubing. In some aspects, Zone 3 outlet 18075 can also be the inlet of Zone 4. In some aspects, Zone 4 inlet 18075 is in fluidic communication with container 18088 via tubing. In some aspects, container 18088 contacts plate 18086, which can be a temperature-controlled plate. In some aspects, inlet 18090 can be used to pressure test Zone 4 and Zone 3. In some aspects, container 18088 is in fluidic communication with filters 18084, such as 40 μm aggregate filters, and further in fluidic communication with Zone 4 outlet 18092 via tubing. In some aspects, Zone 4 outlet 18092 can also be Zone 5 inlet. In some aspect, Zone 4 outlet 18092 is in fluidic communication with container 18100 via tubing. In some aspects, container 18100 can be in contact with plate 18098, which can be a temperature-controlled plate. In some aspects, container 18106 is in contact with plate 18104, which can be a temperature-controlled plate. In some aspects, container 18110 can contact plate 18108, which can be a temperature controlled plate. In some aspects, inlet 18102 can be used to pressure test Zone 5. In some aspects, container 18100 is in fluidic communication with cell suspension device 18094, which can be a TFF filter assembly. In some aspects, cell suspension device 18094 is in fluidic communication with container 18100 via tubing. In some aspects, container 18100 is in fluidic communication with filter 18096, which can be a 40 μm aggregate filter, and further fluidically connected to Zone 5 outlet 18112 via tubing. In some aspects, Zone 5 outlet 18112 is also the inlet to Zone 6.

In some aspects, Zone 5 outlet 18112 is in fluidic communication with container 18126 via tubing. In some aspects, container 18126 contacts scale system 18122. In some aspects, container 18126 can further be in contact with backlight illumination 18124. In some aspects, container 18134 is in contact with plate 18132, which can be a temperature-controlled plate. In some aspects, container 18134 is in fluidic communication with container 18126 via tubing. In some aspect, container 18126 is in fluidic communication with filter 18120, which can be a 40 μm aggregate filter. In some aspects, inlet 18118 can be in fluidic communication with filter 18120. In some aspects, inlet 18118 can be used to pressure test Zone 6. In some aspects, container 18126 is in fluidic communication with containers 18130 via tubing. In some instances, container 18130 are in contact with scale system 18128. In some aspects, waste from Zone 5 or Zone 6 can flow via tubing into waste container 18116, which can be in contact with scale system 18114.

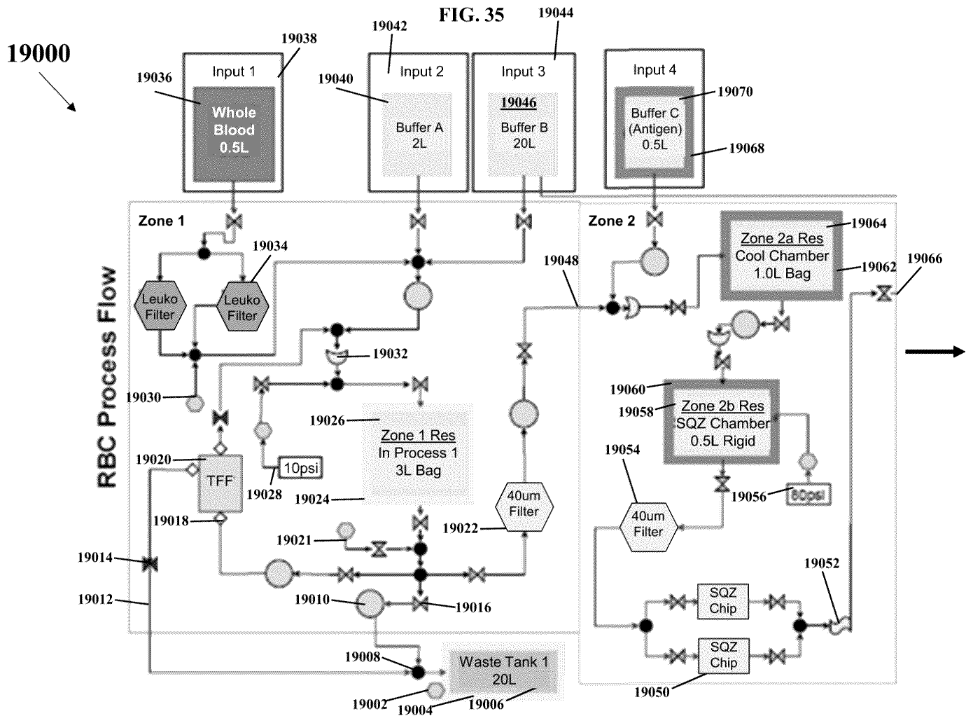

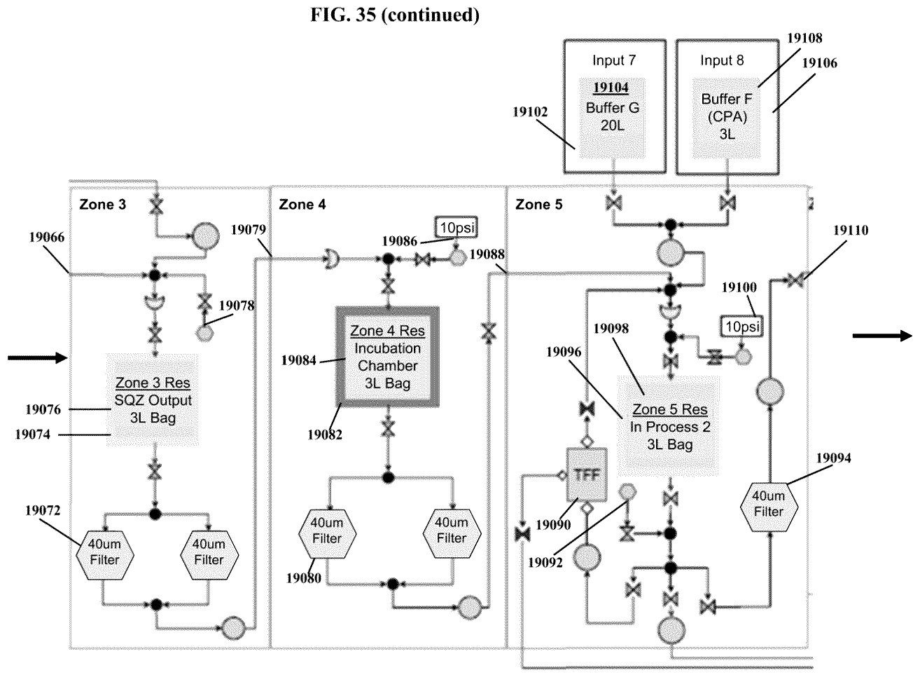

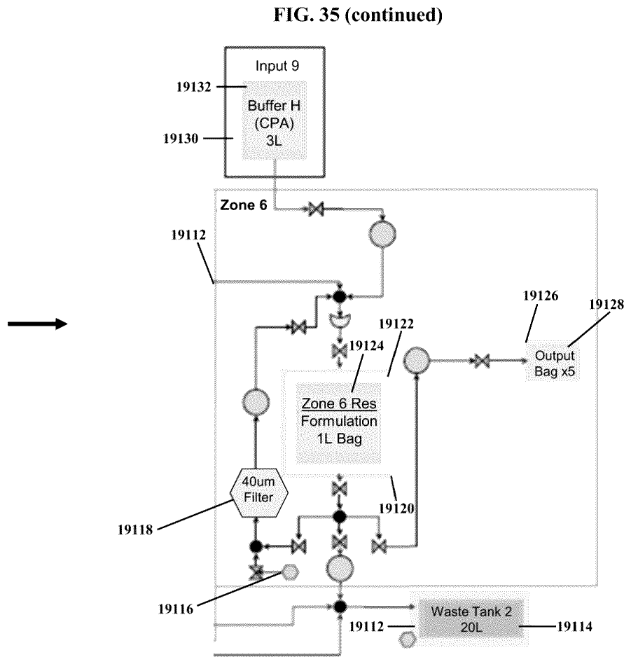

Referring now to FIG. 35, in some aspects, a point of care system process flow is a flow as presented in 19000. It is noted that solid black lines 19012 represent flow paths. Grey hexagons 19002 represent filters, which in some aspects are air filters. Black circles 19008 represent tubing junctions. Grey circles 19010 represent pumps. Black bowties 19014 represent valves, which in some aspects can be proportional pinch valves. Grey bowties 19016 represent valves, which can be in some aspects pinch valves. White diamonds 19018 represent pressure gauges. Grey crescents 19032 represent bubble sensors. Wavy rectangles 19052 represent flow sensors. In some aspects, Container 19036 contacts plate 19038, which can be a temperature-controlled plate. In some aspects, container 19036 is in fluidic communication with cell isolation devices 19034, such as leukoreduction filters, and further in fluidic communication with container 19026 via tubing. In some aspects, container 19026 is in contact with scale system 19024. In some aspects, inlet 19030 is in fluidic communication with container 19026 via tubing. In some aspects, inlet 19028 can be used to pressure test Zone 1. In some aspects, inlet 19021 is in fluidic communication with container 19026 via tubing. Container 19026 can further be in fluidic communication with cell suspension device 19020, such as a TFF filter assembly. In some aspects, container 19040 contacts plate 19042, which can be a temperature-controlled plate. Container 19040 can be in fluidic communication with container 19026 via tubing. In some aspects, container 19046 contacts plate 19044, which can be a temperature-controlled plate. Container 19046 can further be in fluidic communication with container 19026 via tubing. In some aspects, container 19026 is in fluidic communication with aggregate filter 19022, such as a 40 μm aggregate filter, and further in fluidic communication with Zone 1 outlet 19048 via tubing. In some aspects, waste from Zone 1 can flow via tubing into waste container 19006, which can be in contact with scale system 19004.

Further referring to FIG. 35, Zone 1 outlet 19048 can be a Zone 2 inlet. In some aspects, Zone 1 outlet 19048 is in fluidic communication with container 19064. Container 19064 can contact plate 19062, which can be a temperature-controlled plate. In some aspects, container 19070 contacts plate 19068, which can be a temperature-controlled plate. In some aspects, container 19070 is in fluidic communication with container 19064 via tubing. In some aspects, container 19064 is in fluidic communication with container 19058 via tubing. In some instances, container 19058 contacts plate 19060, which can be a temperature-controlled plate. In some aspects, inlet 19056 can be used to pressure test Zone 3. In some aspects, container 19058 is in fluidic communication with filter 19054, such as a 40 μm aggregate filter, and cell deformation devices 19050 via tubing. In some aspects, cell deformation devices 19050 are in fluidic communication with Zone 2 outlet 19066, can be an inlet to Zone 3. In some aspects, inlet 19066 is in fluidic communication with container 19076 via tubing. In some aspects, container 19076 contacts scale system 19074. In some aspects, inlet 19078 is in fluidic communication with container 19076 via tubing. In some aspects, container 19076 is in fluidic communication with filters 19072, such as 40 μm aggregate filters, and further fluidically connected to Zone 3 outlet 19079 via tubing. In some aspects, Zone 3 outlet 19079 can be an inlet to Zone 4. In some aspects, Zone 3 outlet 19079 is in fluidic communication with container 19084 via tubing. In some aspects, container 19084 contacts plate 19082, which can be a temperature-controlled plate. In some aspects, inlet 19086 can be used to pressure test Zone 4 and Zone 3. In some aspects, container 19084 is in fluidic communication with filters 19080, such as 40 μm aggregate filters, and further in fluidic communication with Zone 4 outlet 19088 via tubing. Zone 4 outlet 19088 can be an inlet to Zone 5. In some aspects, Zone 4 outlet 19088 is in fluidic communication with container 19098. Container 19098 can contact scale system 19096. In some aspects, container 19098 is in fluidic communication with container 19098 via tubing. In some aspects, container 19104 contacts plate 19102, which can be a temperature-controlled plate. In some aspects, container 19108 contacts plate 19106, which can be a temperature-controlled plate. In some aspects, container 19108 is in fluidic communication with container 19098 via tubing. In some aspects, inlet 19100 can be used to pressure test Zone 5. In some aspects, container 19098 is in fluidic communication with cell suspension device 19090, such as a TFF filter assembly, via tubing. In some aspects, inlet 19092 is in fluidic communication with container with cell suspension device 19090 via tubing. In some aspects, container 19098 is further in fluidic communication with aggregate filter 19094, which can be a 40 μm aggregate filter, and further in fluidic communication with Zone 5 outlet 19110.

In some aspects, Zone 5 outlet 19110 is in fluidic communication with container 19124 via tubing. In some aspects, container 19124 contacts scale system 19120. In some aspects, container 19124 can further be in contact with backlight illumination 19122. In some aspects, container 19132 is in contact with plate 19130, which can be a temperature-controlled plate. In some aspects, container 19132 is in fluidic communication with container 19124 via tubing. In some aspect, container 19124 is in fluidic communication with filter 19118, which can be a 40 μm aggregate filter. In some aspects, inlet 19116 can be in fluidic communication with filter 19118. In some aspects, inlet 19116 can be used to pressure test Zone 6. In some aspects, container 19124 is in fluidic communication with containers 19128 via tubing. In some instances, container 19128 are in contact with scale system 19126. In some aspects, waste from Zone 5 or Zone 6 can flow via tubing into waste container 19114, which can be in contact with scale system 19112.

Referring now to FIG. 27, in some aspects, a PBMC process flow is a flow as presented in 12000. It is noted that flow paths 12006 are represented by solid black lines; optional flow paths 12021 are represented by black dashed lines; and waste paths 12004 are represented as grey dashed lines. Solid black circles 12002 represent switch valves. Grey bowties 12010 represent valves. Grey circles 12012 represent pumps. In some aspects, containers such as 12030, 12038, 12044, and 12048 can be fluidically connected to Zone 1 via tubing and pumps, such as pump 12028 for container 12030 or pumps 12012 for containers 12038. In some aspects, container 12030 can be in contact with plate 12033, which can be a temperature-controlled plate. In some aspects, container 12030 can be releasably coupled to fastening element 12032, which fastening element can be a hook and can also function as a part of a scale system. In some aspects, sample from container 12030 can be pumped by pump 12028 through tubing to aggregate filter 12029. Aggregate filter 12029 can be in fluidic communication with container 12024 by tubing and valves. In some aspects, container 12038 is in contact with plate 12036, which can be a temperature-controlled plate. In some aspects, container 12038 is releasably coupled to fastening element 12034, which can function as a part of a scale system. In some aspects, container 12044 is in contact with plate 12042, which can be a temperature-controlled plate. In some aspects, container 12044 is releasably coupled to fastening element 12046, which can function as a part of a scale system. In some aspects, container 12044 is in contact with plate 12040. In some aspects, container 12050 is in contact with plate 12048, which can be a temperature-controlled plate. In some aspects, container 12050 is releasably coupled to fastening element 12052, which can function as a part of a scale system. In some aspects, containers 12038, 12044, and 12050 are fluidically connected to Zone 1 via tubing and valves. In some aspects, container 12050 is further fluidically connected to Zone 2 via tubing and valves. In some aspects, each of containers 12038, 12044, and 12050 are in fluidic communication with container 12024 through tubing and valves. In some aspects, container 12024 is in fluidic communication with sort chips 12020 and TFF filter assembly 12018 through tubing and valves. Fluid from container 12024 can be pumped by pumps 12012 throughout Zone 1. In some aspects, TFF filter assembly 12018 is in fluidic communication with pressure gauges 12008, with bubble sensor 12026, and additionally with a flow sensor. In some aspects, zone 1 comprises one or more sort chips 12020, such as two sort chips 12020. In some aspects, container 12022 is in fluidic communication with aggregate filter 12016 and flow sensor 12014, and further in fluidic communication with Zone 2 via outlet 12054.

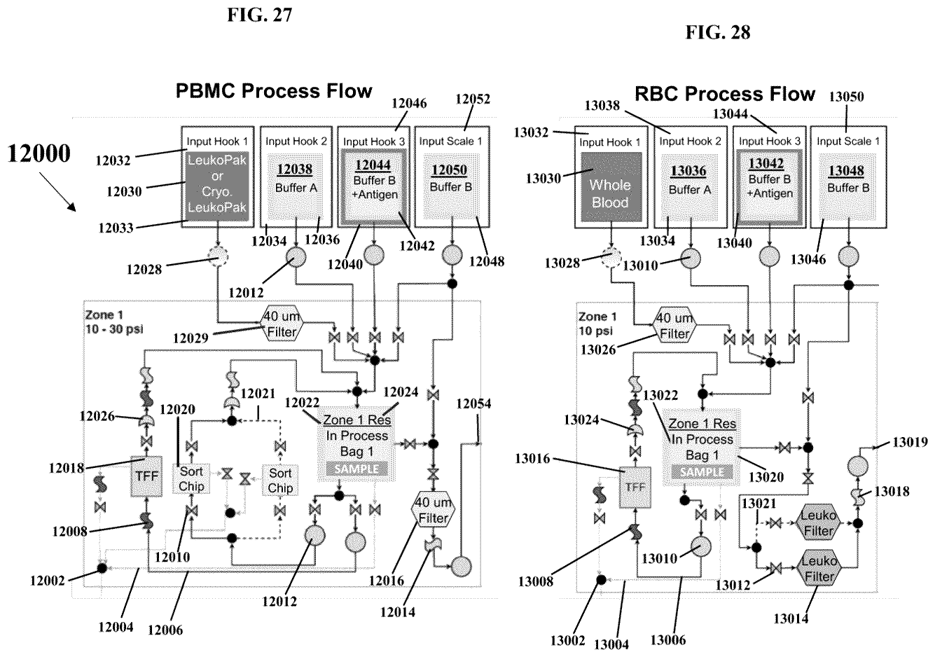

Referring now to FIG. 28, in some aspects, a RBC process flow is presented in 13000. It is noted that flow paths 13006 are represented by solid black lines; optional flow paths 13021 are represented by black dashed lines; and waste paths 13004 are represented as grey dashed lines. Solid black circles 13002 represent switch valves. Grey bowties 13012 represent valves. Grey circles 13010 represent pumps. Crescents 13024 represent bubble sensors. Black wavy rectangles 13008 represent pressure gauges. Grey wavy rectangles 13018 represent flow sensors. In some aspects, container 13030 is in contact with plate 13031, which can be a temperature-controlled plate. In some aspects, container 13030 is releasably coupled to fastening element 13032, which can also function as a part of a scale system. In some aspects, container 13030 is in fluidic communication with pump 13028. In some aspects, container 13030 is in fluidic communication with an aggregated filter 13026 and further fluidically connected to container 13022. Container 13036 can be in contact with plate 13034, which plate can be a temperature-controlled plate. Container 13038 can be releasably coupled to fastening element 13038, which can in some instances be a hook. In some aspects, container 13042 is in contact with plate 13040, which can be a temperature-controlled plate. In some aspects, container 13042 is releasably coupled to fastening element 13044. In some aspects, container 13048 is in contact with plate 13046, which can be a temperature-controlled plate. In some aspects, container 13048 is releasably coupled to fastening element 13050. In some aspects, each of containers 13030, 13036, 13042, and 13048 are in fluidic communication with Zone 1. In some aspects, container 13048 is further in fluidic communication with Zone 2. In some aspects, container 13033 is in contact with plate 13020, which can be a temperature-controlled plate. In some aspects, container 13022 is in fluidic communication with TFF filter 13016. Container 13022 is further in fluidic communication with leukoreduction filter 13014 and also to Zone 2 via outlet 13019.

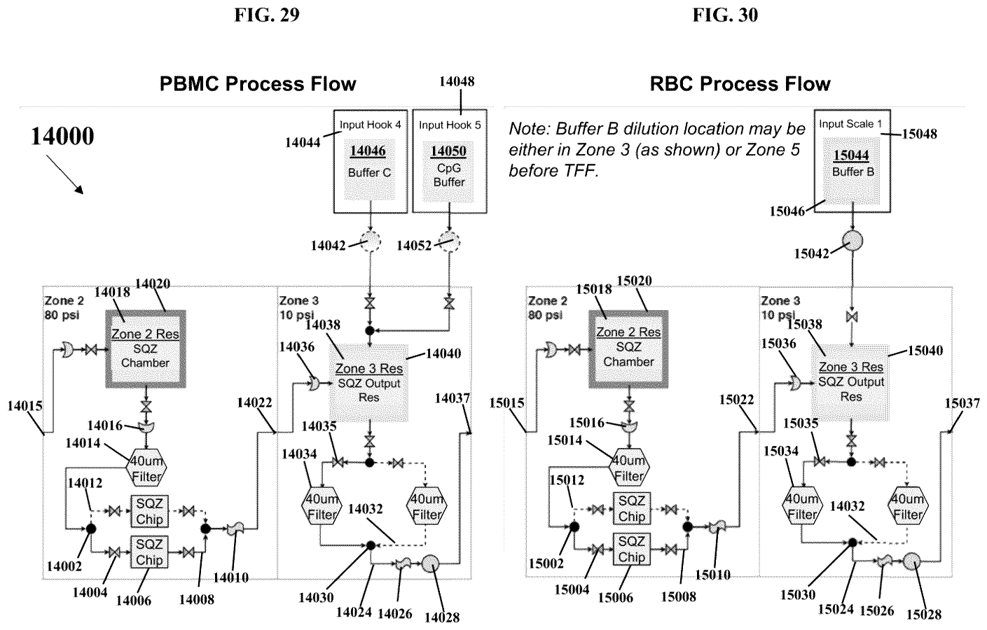

Referring now to FIG. 29, in some aspects, a PBMC process flow 14000 for Zone 2 and Zone 3 is presented. It is noted that crescents 14016 represent bubble sensors; solid black circles 14002 represent switch valves; bowties 14004 represent valves; grey wavy triangles 14010 represent flow sensors. Solid black lines 14008 represent flow paths. Dashed black lines 14012 represent optional flow paths. Fluid from Zone 1 can enter Zone 2 via inlet 14015, which is in fluidic communication with container 14018. Container 14018 can be in contact with plate 14020, which can be a temperature-controlled plate. Container 14018 is further in fluidic communication with a cell aggregate filter 14014 and to cell deformation device 14006, such as microfluidic chips for cell deformation. In some aspects, cell deformation device 14006 is in fluidic communication with Zone 3 via outlet 14022. Referring to Zone 3: solid black lines 14024 represent flow paths; solid black circles 14030 represent switch valves; wavy grey rectangles 14026 represent flow sensors; dotted grey lines 14032 represent optional flow paths; bowties 14035 represent valves; crescents 14036 represent bubble sensors; grey circles 14028 represent pumps. In some aspects, fluid from Zone 2 enters Zone 3 via outlet 14022 from Zone 2, which serves as the inlet for Zone 3, and flows into container 14038. Container 14038 is in contact with plate 14040, which can be a temperature-controlled plate. In some aspects, container 14038 is in fluidic communication with aggregate filter 14034 and further in fluidic communication with Zone 4 via outlet 14037. Furthermore, container 14046 can be in fluidic communication with Zone 3, such that fluid from container 14046 can be pumped by pump 14042 into Zone 3. In some aspects, container 14046 is releasably coupled to a fastening element 14044. In some aspects, container 14050 is in fluidic communication with Zone 3 such that pump 14052 can pump fluid from container 14050 into Zone 3. In some aspects, container 14050 is releasably coupled to fastening element 14048.

Referring now to FIG. 30, in some aspects, a RBC process flow 15000 for Zone 2 and Zone 3 is presented. It is noted that crescents 15016 represent bubble sensors; solid black circles 15002 represent switch valves; bowties 15004 represent valves; grey wavy triangles 15010 represent flow sensors. Solid black lines 15008 represent flow paths. Dashed black lines 15012 represent optional flow paths. Fluid from Zone 1 can enter Zone 2 via inlet 15015, which is in fluidic communication with container 15018. Container 15018 can be in contact with plate 15020, which can be a temperature-controlled plate. Container 15018 is further in fluidic communication with a cell aggregate filter 15014 and to cell deformation device 15006, such as microfluidic chips for cell deformation. In some aspects, cell deformation device 15006 is in fluidic communication with Zone 3 via outlet 15022. Referring to Zone 3: solid black lines 15024 represent flow paths; solid black circles 15030 represent switch valves; wavy grey rectangles 15026 represent flow sensors; dotted grey lines 15032 represent optional flow paths; bowties 15035 represent valves; crescents 15036 represent bubble sensors; grey circles 15028 represent pumps. In some aspects, fluid from Zone 2 enters Zone 3 via outlet 15022 from Zone 2, which serves as the inlet for Zone 3, and flows into container 15038. Container 15038 is in contact with plate 15040, which can be a temperature-controlled plate. In some aspects, container 15038 is in fluidic communication with aggregate filter 15034 and further in fluidic communication with Zone 4 via outlet 15037. In some aspects, container 15044 can be in fluidic communication with Zone 3, such that fluid from container 15044 can be pumped by pump 15042 into Zone 3. In some aspects, container 15044 is in contact with plate 15046, which can be a temperature-controlled plate. In some aspects, container 15044 can be in contact with scale 15048.

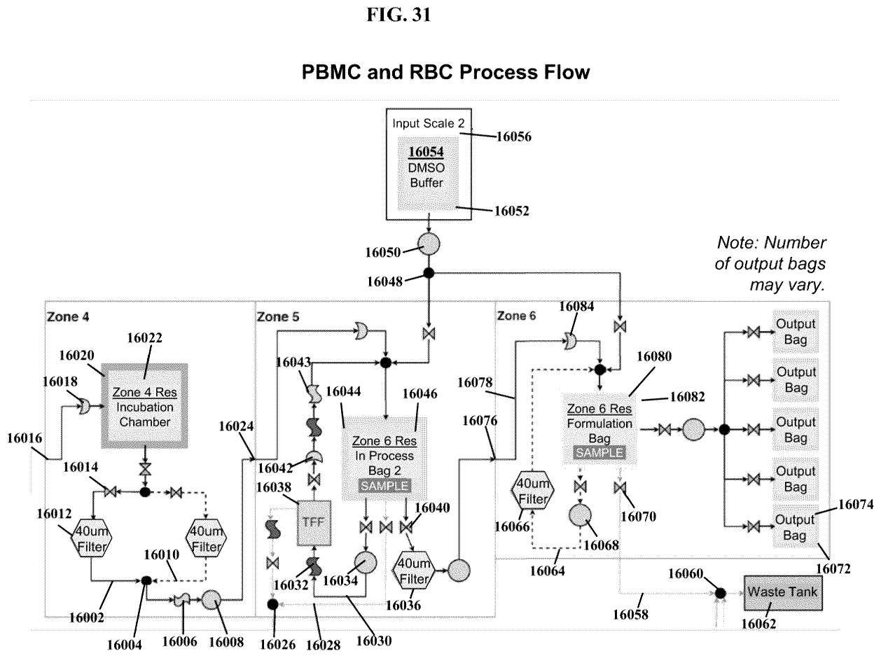

Referring now to FIG. 31, in some aspects, a PBMC or an RBC process flow can be process flow 16000. Referring to Zone 4, solid black line 16002 represents flow path; solid black circles 16004 represent switch valves; grey circles 16008 represent pumps; grey wavy rectangles 16006 represent flow sensors; bowties 16014 represent valves; crescents 16018 represent bubble sensors; and black dashed line 16010 represents an optional flow path. In some aspects, fluid enters Zone 4 via inlet from Zone 3 16016 and flows into container 16022. Container 16022 is in contact with plate 16020, which can be a temperature-controlled plate. In some aspects, container 16022 is in fluidic communication with aggregate filter 16012 and further in fluidic communication with Zone 5 via Zone 4 outlet/Zone 5 inlet 16024.

Referring to Zone 5, solid black circles 16026 represent switch valves; wavy black rectangle 16032 represent pressure gauges; crescents 16042 represent bubble sensors; grey circles 16034 represent pumps; bowties 16040 represent valves; and wavy grey rectangles 16043 represent flow sensors; black solid lines 16030 represent flow path; grey dashed line 16028 represents a waste flow path. In some aspects, fluid from Zone 4 enters Zone 5 via inlet 16024 and flows to container 16046. Container 16046 is in contact with plate 16044, which can be a temperature-controlled plate. In some aspects, container 16044 is in fluidic communication with TFF filter assembly 16038. Furthermore, container 16044 is in fluidic communication with aggregate filter 16036 and further in fluidic communication with Zone 6 via outlet to Zone 6/inlet to Zone 6 16076.

Referring now to Zone 6, grey dashed lines 16058 represent waste flow paths; black solid lines 16078 represent flow paths; dashed black lines 16064 represent optional flow paths; grey circles 16068 represent pumps; black circles 16060 represent switch valves; bowties 16070 represent valves; crescents 16084 represent bubble sensors. In some aspects, fluid from Zone 5 enters Zone 6 via inlet to Zone 6 16076 and flows into container 16080. In some aspects, container 16080 is in contact with plate 16082, which can be a temperature-controlled plate. In some aspects, container 16080 is in fluidic communication with aggregate filter 16066. In some aspects, container 16080 is in fluidic communication with containers 16074. Containers 16074 can each rest on a respective plate 16072, which can be a temperature-controlled plate and further which can be a part of a scale system. Furthermore, Zone 6 can comprise waste tank 16062, into which waste flows along waste flow path 16058. Waste tank 16062 can be in contact with a scale system. In some aspects, the scale system comprises a tension load cell or a compression load cell. Further referring to FIG. 31, container 16054 can be in contact with plate 16052, which can be a temperature-controlled plate. In some aspects, container 16054 is fluidically connected to Zone 5 and to Zone 6, such that pump 16050 can pump fluid into either zone as directed by switch valve 16048. In some aspects, container 16054 is in contact with scale 16056.

### 2. Methods for Processing Cells

In some aspects, the present disclosure generally relates to a method for processing cells, wherein the method is performed by a system comprising one or more of a suspension preparation subsystem, a cell-deformation subsystem, a dilution subsystem, an incubation subsystem, a cell-washing subsystem, and a container-filling subsystem, the method comprising: i. at the suspension preparation subsystem: 1. receiving cells from a container; 2. optionally performing a cell isolation operation on the cells thereby producing isolated cells; 3. receiving a delivery media via a delivery media inlet; and 4. producing a cell suspension by suspending the cells or the isolated cells to in the delivery media, thereby producing a cell suspension; ii. at the cell-deformation subsystem: 1. receiving flow of the cell suspension from the suspension preparation subsystem; 2. flowing the cell suspension through one or more cell-deforming constrictions configured to cause perturbations in cell membranes of the cells to allow entry of a payload into the cell, thereby producing a cell suspension comprising engineered cells; iii. at the dilution subsystem: 1. receiving flow of the cell suspension comprising engineered cells from the cell-deformation subsystem; 2. receiving a fluid or dry reagent via a buffer inlet; and producing a diluted cell suspension by mixing the cell suspension with the fluid or dry reagent; iv. at the incubation subsystem: 1. receiving flow of the incubated cell suspension from the incubation subsystem; 2. performing a buffer exchange operation on the cells to suspend the cells in a preservation media; and vi. at the container filling subsystem: receiving flow of the cells suspended in preservation media from the cell-washing subsystem; and introducing the cells suspended in preservation media into one or more containers.

In some aspects, the system for processing cells processes red blood cells (RBC). In some aspects, the system for processing cells processes peripheral blood mononuclear cells (PBMCs). In some aspects, the system for processing cells processes activating antigen carrier (AAC) cells. In some aspects, the system for processing cells processes tolerizing antigen carrier (TAC) cells. In some aspects, the system for processing cells process antigen presenting cells (APCs). In some aspects, the system for processing cells processes T cells. In some aspects, the system for processing cells processes B cells. In some aspects, the system for processing cells processes macrophages. In some aspects, the system for processing cells processes natural killer (NK) cells. In some aspects, the system for processing cells processes dendritic cells. In some aspects, the system for processing cells processes immune cells. In some aspects, the system for processing cells processes monocytes. In some aspects, the system for processing cells processes monocytes leukocytes. In some aspects, the system for processing cells processes eosinophils. In some aspects, the system for processing cells processed basophils. In some aspects, the system for processing cells processes natural killer T (NKT) cells. In some aspects, the system for processing cells processes mast cells. In some aspects, the system for processing cells processes neutrophils. In some aspects, the method removes clotting plasma and/or serum. In some aspects, the method is performed in about 5 to about 7 hours. In some aspects, the method produces cell therapeutics. In some aspects, the payload comprises one or more reprogramming factors. In some aspects, the payload comprises one or more nucleic acids. In some aspects, the payload comprises one or more differentiation factors. In some aspects, the payload comprises one or more neuron reprogramming factors. In some aspects, a cell isolation operation is not performed as a part of the method for processing cells. In some aspects, a cell isolation operation is not performed as a part of the process for processing TACs. In some aspects, a cell isolation operation is not performed as a part of the process for processing APCs. In some aspects, a cell isolation operation is not performed as a part of the process for processing PBMCs.

### 3. Kits

In some aspects, the present disclosure generally relates to a kit for use in a system for processing blood, the kit comprising one or more of: i. a first kit comprising releasably couplable components configured to be releasably coupled to a frame of a suspension preparation subsystem of the system, wherein the first set of releasably couplable components comprises: 1. a cell isolation device; or 2. a tangential flow filtration membrane assembly; ii. a second kit comprising releasably couplable components configured to be releasably couplable to a frame of a cell-deformation subsystem of the system, wherein the second set of releasably couplable components comprises one or more microfluidic chips comprising one or more cell-deforming constrictions through which cells may be forced to cause perturbation of membranes of the cells; iii. a third kit comprising releasably couplable components configured to be releasably couplable to a frame of a dilution subsystem of the system, wherein the third set of releasably couplable components comprises at least one cell aggregate filter; iv. a fourth kit comprising releasably couplable components configured to be releasably couplable to a frame of an incubation subsystem of the system, wherein the fourth set of releasably couplable components comprises at least one cell aggregate filter; v. a fifth kit comprising releasably couplable components configured to be releasably couplable to a frame of a cell-washing subsystem of the system, wherein the fifth set of releasably couplable components comprises a second tangential flow filtration membrane assembly; and vi. a sixth kit comprising releasably couplable components configured to be releasably couplable to a frame of a container-filling subsystem of the system, wherein the sixth set of releasably couplable components comprises at least one cell aggregate filter.

In some aspects, one or more of the releasably couplable components of the second kit of releasably couplable components is configured to be fluidly connected to one or more of the releasably couplable components of the first kit of releasably couplable components. In some aspects, one or more of the releasably couplable components of the third kit of releasably couplable components is configured to be fluidly connected to one or more of the releasably couplable components of the second set of releasably couplable components. In some aspects, one or more of the releasably couplable components of the fourth kit of releasably couplable components is configured to be fluidly connected to one or more of the releasably couplable components of the third kit of releasably couplable components. In some aspects, one or more of the releasably couplable components of the fifth kit of releasably couplable components is configured to be fluidly connected to one or more of the releasably couplable components of the fourth kit of releasably couplable components. In some aspects, one or more of the releasably couplable components of the sixth kit of releasably couplable components is configured to be fluidly connected to one or more of the releasably couplable components of the fifth kit of releasably couplable components.

In some aspects, one or more of the components of one or more of the kits are configured to be fluidly connected to one or more components of the corresponding subsystem of the system. In some aspects, the kit comprises the first, second, third, fourth, fifth, and sixth kits. In some aspects, each kit is packaged separately. In some aspects, at least two kits are packaged together.

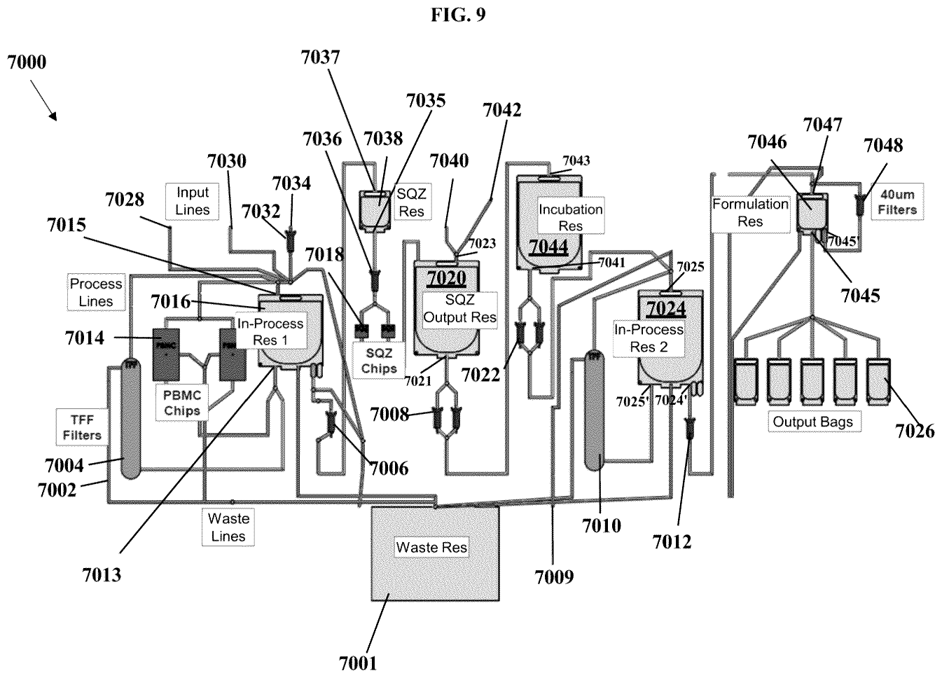

In some aspects, a disposable kit for use with the point of care system comprises disposable kit 7000 of FIG. 9. Referring now to FIG. 9, in some instances, kit 7000 comprises input lines 7028, 7030, and 7034, which can be used, for instance, to deliver delivery media, buffer, or sample. Inlet 7028 and 7030 can be fluidically connected to container 7016 via tubing and inlet 7015. In some aspects, inlet 7034 is fluidically connected to aggregate filter 7032, which is fluidically connected to container 7016. In some aspects, container 7016 is fluidically connected to TFF filter assembly 7004 via tubing. In some instances, kit 7000 comprises TFF filter assembly 7004, which is fluidically connected via tubing 7002 to cell isolation device 7014. In some aspects, cell isolation device 7014 are fluidically connected to container 7016 via outlet 7013 and tubing. Container 7016 can be fluidically connected to aggregate filter 7006, which is fluidically connected to container 7038 via tubing. In some aspects, container 7038 comprises inlet 7037 and outlet 7035, which are coupled to tubing. In some aspects, container 7038 is fluidically connected to aggregate filter 7036 via outlet 7035 and tubing, which is further connected to cell deformation devices 7018. In some aspects, cell deformation devices 7018 are fluidically connected to container 7020 via tubing. Inlet 7040 and 7042 are fluidically connected to container 7020 via tubing and inlet 7023. Container 7020 is fluidically connected to aggregate filters 7008 via outlet 7021 and tubing, which are fluidically connected to container 7044 via tubing and inlet 7043. In some aspects, container 7044 is fluidically connected to aggregate filters 7022 via outlet 7041 and tubing, which are fluidically connected to container 7024 via tubing and inlet 7025. In some aspects, inlet 7009 is fluidically connected to container 7024 via tubing and inlet 7025. In some aspects, container 7024 is fluidically connected via outlet 7025′ and tubing to TFF assembly 7010, which can be further fluidically connected to container 7024 via tubing and inlet 7025. In some aspects, container 7024 can be fluidically connected via outlet 7024 to aggregate filter 7012, which is fluidically connected to container 7046 via tubing and inlet 7047. In some aspects, container 7047 is fluidically connected via outlet 7045′ to aggregate filter 7048, which can be fluidically connected to container 7046 via tubing and inlet 7047. Container 7046 can further be fluidically connected to out containers 7026 via outlet 7045 and tubing. In some aspects, kit 7000 can further comprise waste container 7001, which is coupled to tubing.

Contrastingly, in some aspects, a separate kit can be used for each subsystem, as discussed below. Each kit comprises components releasably couplable to the frame of the system, and further couplable to the corresponding subsystem of the system.

In some aspects, a disposable kit for use with the point of care system is sterile. In some aspects, a disposable kit for use with the point of care system is assembled in a clean room environment. In some aspects, gamma radiation is used to sterilize a disposable kit for use with the point of care system. In some aspects, ethanol is used to sterilize a disposable kit for use with the point of care system. In some aspects, a disposable kit for use with the point of care system is packaged in a clean room. In some aspects, a disposable kit for use with the point of care system is packaged in a thermoformed tray.

a. Suspension Preparation Subsystem (Zone 1)

In some aspects, the Zone 1 kit comprises the components presented in FIG. 12 or FIG. 13, which are discussed supra.

In some aspects, the kit comprises a first kit comprising a suspension preparation subsystem kit. In some aspects, the first kit of releasably couplable components comprises a cell aggregate filter. In some aspects, the first kit of releasably couplable components comprises a leukoreduction filter. In some aspects, the first kit of releasably couplable components comprises a container. In some aspects, the first kit of releasably couplable components comprises a tangential flow filtration filter assembly. In some aspects, the first kit of releasably couplable components comprises one or more of: tubing, a tube fitting, a connector, a clamp, a sampling bulb, a carboy, an air filter, and a tangential flow filtration filter assembly.

b. Cell Deformation Subsystem (Zone 2)

In some aspects, the Zone 2 kit comprises the components presented in FIG. 14, which is discussed supra.

In some aspects, the kit comprises a second kit comprising a cell deformation subsystem. In some aspects, the second kit of releasably couplable components comprises a rigid sample vessel. In some aspects, the second kit of releasably couplable components comprises a cell-aggregate filter. In some aspects, the second kit of releasably couplable components comprises preparation vessel. In some aspects, the second kit of releasably couplable components comprises one or more microfluidic chips. In some aspects, the second kit of releasably couplable components comprises one or more microfluidic chip cartridges. In some aspects, the second kit of releasably couplable components comprises one or more of: tubing, a tube fitting, a connector, a clamp, a container, a bag, an air filter, and a barrel filter.

c. Dilution Subsystem (Zone 3)

In some aspects, the Zone 3 kit comprises the components presented in FIG. 15, which is discussed supra.

In some aspects, the kit comprises a third kit comprising a dilution subsystem. IN some aspects, the third kit of releasably couplable components comprises a container. In some aspects, the third kit of releasably couplable components comprises a cell aggregate filter. In some aspects, the third kit of releasably couplable components comprises one or more of: tubing, a tube fitting, a connector, a clamp, a sampling bulb, and a carboy.

d. Incubation Subsystem (Zone 4)

In some aspects, the Zone 4 kit comprises the components presented in FIG. 16, which is discussed supra.

In some aspects, the kit comprises a fourth kit comprising an incubation subsystem. In some aspects, the fourth kit of releasably couplable components comprises a cell aggregate filter. In some aspects, the fourth kit of releasably couplable components comprises one or more containers. In some aspects, the fourth kit of releasably couplable components comprises one or more of: tubing, a tube fitting, a connector, and a clamp.

e. Cell Washing Subsystem (Zone 5)

In some aspects, the Zone 5 kit comprises the components presented in FIG. 17, which is discussed supra.

In some aspects, the kit comprises a fifth kit comprising a cell washing subsystem. In some aspects, the fifth kit of releasably couplable components comprises a cell aggregate filter. In some aspects, the fifth kit of releasably couplable components comprises a container. In some aspects, the fifth kit of releasably couplable components comprises a tangential flow filtration filter assembly. In some aspects, the fifth kit of releasably couplable components comprises one or more of: tubing, a tube fitting, a connector, a clamp, a sampling bulb, an air filter, and a carboy.

f. Container Filling Subsystem (Zone 6)

In some aspects, the Zone 6 kit comprises the components presented in FIG. 18, which is discussed supra.

In some aspects, the kit comprises a sixth kit comprising a container filling subsystem. In some aspects, the sixth kit of releasably couplable components comprises at least one container. In some aspects, the container is a cryopreservation bag. In some aspects, the sixth kit of releasably couplable components comprises a cell aggregate filter. In some aspects, the sixth kit of releasably couplable components comprises one or more of: tubing, a tube fitting, a connector, a clamp, and a sampling bulb.

g. Temperature Control Subsystem

In some aspects, the temperature control subsystem kit comprises components to perform the functions otherwise performed by the dilution subsystem (Zone 3) and incubation subsystem (Zone 4), as described supra. For instance, the temperature control subsystem kit can comprise a container for receiving cell suspension comprising engineered cells from the cell deformation subsystem (Zone 2) and one or more inlets for introducing fluids and/or dry reagents to container comprising the cell suspension. The container, when attached to the frame of the system, can contact a temperature-controlled plate.

In some aspects, the kit comprises a temperature control subsystem kit. In some aspects, the temperature control kit comprises a container. In some aspects, the temperature control kit components comprises a cell aggregate filter. In some aspects, the temperature control kit comprises one or more of: tubing, a tube fitting, a connector, a clamp, a sampling bulb, and a carboy.

h. Waste Container Kit

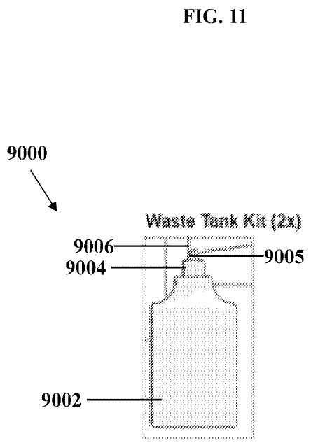

In some aspects, the kit comprises a waste container kit, such as waste container kit 9000. Referring now to FIG. 11, waste container kit 900 comprises waste container 9002, which is coupled to cap 9004. Cap 9004 can be coupled to tubing 9006, such as through outlet 9005.

i. Kit Installation

In some aspects, a kit comprising one or more of the kits for Zone 1 Zone 6 can be packaged in an accordion tray package. In some aspects, such a kit can be installed by coupling it to the frame of the system, as presented in FIG. 22. Referring to FIG. 22, kit 10002 can be releasably coupled to frame 10004, such as by attached a panel to the device, which comprises one or more subsystems. Remaining subsystems can be attached by unfolding accordion tray kit 10002. For instance, subsystems comprised on panel 10006 of kit 10002 can be releasably connected to frame 10004 by unfolding the accordion tray. In some aspects, panel 10010 can comprise one or more subsystems, which can be connected to frame 10004 by unfolding the panel.

In some aspects, a kit comprising one or more of the kits for Zone 1 Zone 6 can be packaged as a rollable sheet. In some aspects, such a kit can be installed by coupling it to the frame of the system, as presented in FIG. 23. Referring to FIG. 23, rollable sheet 11002 can be releasably coupled to frame 110004 by unrolling the sheet and connecting it to frame 11004. For instance, partially unrolled sheet 11006 can be further unrolled to be fully unrolled sheet 11008, which is then connected to the frame.

### 4. Further Systems for Processing Cells

A system for processing cells is provided. The system may be configured to receive input of cells, such as in the form of blood or in the form of cells suspended in any suitable media, and to automatically process the cells to perform one or more operations on the cells, such as isolation, suspension in one or more medias, buffer exchange, payload delivery, dilution, incubation, container filling, temperature control, filtering, agitation, pressurization, and/or cryopreservation.

In some embodiments, the system may comprise a plurality of subsystems, wherein each subsystem is configured to perform one or more cell processing operations. In some embodiments, one or more of the subsystems may be in fluid communication with one or more of the other subsystems, such that fluid (e.g., a cell suspension) may automatically flow (e.g., gravitationally and/or under pressure) from one subsystem to the next. In some embodiments, the system may be configured to cause the cells being processed to pass from one subsystem to the next (and/or from a system input to a system output) without manual (e.g., physical) human intervention. In some embodiments, one or more of the subsystems may be configured to be able to be controlled (e.g., temperature, pressure, etc.) independently of one or more of the other subsystems.

In some embodiments, the system may comprise a suspension preparation subsystem configured to receive cells, to perform a cell isolation operation on the cells, to receive a delivery media, and/or to create a cell suspension by causing the cells to be suspended in the delivery media. The cells suspended in the delivery media may then flow from the suspension preparation subsystem to a cell deformation subsystem.

In some embodiments, the cell deformation subsystem may be configured to cause the cell suspension to flow through one or more cell-deforming constrictions configured to cause perturbations in cell membranes of the cells to allow entry of a payload into the cell. After perturbation and/or delivery of the payload, the cell suspension may then flow from the cell deformation subsystem to a dilution subsystem.

In some embodiments, the dilution subsystem may be configured to create a diluted cell suspension by mixing the received cell suspension with a buffer. After dilution of the cell suspension, the diluted cell suspension may then flow from the dilution subsystem to an incubation subsystem.

In some embodiments, the incubation subsystem may be configured to control the temperature of the diluted cell suspension, such as by increasing, decreasing, or maintain the temperature. After incubation, the diluted cell suspension may then flow from the incubation subsystem to a cell-washing subsystem.

In some embodiments, the cell-washing subsystem may be configured to perform a buffer exchange operation on the received diluted cell suspension and to suspend the cells in a media, such as a cryo-protectant media. The cells suspended in the media may then flow from the cell-washing subsystem to a container-filling subsystem.

In some embodiments, the container-filling subsystem may be configured to cause the cells suspended in media to flow into one or more bags, for example for storage and/or preservation such as cryopreservation.

In some embodiments, one or more operations of the system may be configured to be automatically controlled, which may be controlled by one or more processors of the system. Users may execute inputs against one or more user interfaces to control functionalities of the system.

In some embodiments, the system may be configured to receive one or more disposable components that may be inserted into, attached to, or otherwise configured for use with the system. One or more of the disposable components may form all or part of a fluid flow path for cells flowing through the system. Disposable components may be configured for use with a specific subsystem of the system. The disposable components may be provided as one or more kits. In some embodiments, the disposable components may include one or more of: a spiral inertial separation microfluidic consumable, a tangential flow filtration membrane assembly, a cell aggregate filter, a leukoreduction filter, a sampling bulb, tubing, a tube fitting, a connector, a clamp, a bag, and a carboy, a rigid sample vessel, a component comprising one or more cell-deforming constrictions through which cells may be forced to cause perturbation of membranes of the cells, and a cartridge for housing and delivering fluid to and from said component comprising the one or more cell-deforming constructions.

The following exemplary embodiments are provided:

1. A system for processing cells, the system comprising:
a suspension preparation subsystem configured to receive cells, to perform a cell isolation operation on the cells, to receive a delivery media, and to create a cell suspension by causing the cells to be suspended in the delivery media;
a cell-deformation subsystem in fluid communication with the suspension preparation subsystem, wherein the cell-deformation subsystem is configured to receive the cell suspension from the suspension preparation subsystem and to cause the cell suspension to flow through one or more cell-deforming constrictions configured to cause perturbations in cell membranes of the cells to allow entry of a payload into the cell;
a dilution subsystem in fluid communication with the cell-deformation subsystem, wherein the dilution subsystem is configured to receive the cell suspension from the cell-deformation subsystem and to receive a fluid or dry reagent and to create a diluted cell suspension by causing the cell suspension to be mixed with the fluid or dry reagent;
an incubation subsystem in fluid communication with the dilution subsystem, wherein the incubation subsystem is configured to receive the diluted cell suspension from the dilution subsystem and to adjust a temperature of the diluted cell suspension;
a cell-washing subsystem in fluid communication with the incubation subsystem, wherein the cell-washing subsystem is configured to receive the diluted cell suspension from the incubation subsystem and to perform a first buffer exchange operation on the cells to suspend the cells in a preservation media; and
a container-filling subsystem in fluid communication with the incubation subsystem, wherein the container-filling subsystem is configured to receive the cells suspended in preservation media from the cell-washing subsystem and to cause the cells suspended in preservation media to flow into one or more containers.

2. The system of embodiment 1, wherein the suspension preparation subsystem is configured to receive a wash media.

3. The system of any one of embodiments 1-2, wherein the suspension preparation subsystem is configured to receive a dilution media.

4. The system of any one of embodiments 1-3, wherein the suspension preparation subsystem comprises a spiral inertial separation system configured to perform the cell isolation operation on the cells.

5. The system of any one of embodiments 1-4, wherein the suspension preparation subsystem comprises a leukoreduction filter system configured to perform the cell isolation operation on the cells.

6. The system of any one of embodiments 1-5, wherein the suspension preparation subsystem comprises a first tangential flow filtration system configured to perform a second buffer exchange operation.

7. The system of any one of embodiments 1-6, wherein the cell-deformation subsystem comprises a pressurization system configured to generate pressure to force the cell suspension through the one or more cell-deforming constrictions.

8. The system of any one of embodiments 1-7, wherein the cell-deformation subsystem comprises a first temperature control system configured to control a temperature of the cell suspension.

9. The system of any one of embodiments 1-8, wherein the cell-deformation subsystem comprises a first agitation system configured to agitate the cell suspension to promote homogeneity of the cell suspension.

10. The system of any one of embodiments 1-9, wherein receiving the cells at the suspension preparation subsystem comprises one or more of receiving blood and receiving cells suspended in a fluid other than blood.

11. The system of any one of embodiments 1-10, wherein the dilution subsystem comprises a first scale system configured to measure an amount of the fluid or dry reagent added to the cells of the cell suspension.

12. The system of any one of embodiments 1-11, wherein the dilution subsystem comprises a second agitation system configured to agitate the cell suspension to promote homogeneity of the cell suspension.

13. The system of any one of embodiments 1-12, wherein the dilution subsystem comprises a first illumination system configured to illuminate the one or more of the cell suspension and the diluted cell suspension.

14. The system of any one of embodiments 1-13, wherein the incubation subsystem comprises a temperature control device configured to adjust a temperature of the diluted cell suspension.

15. The system of any one of embodiments 1-14, wherein the dilution subsystem comprises a third agitation system configured to agitate the diluted cell suspension to promote homogeneity of the diluted cell suspension.

16. The system of any one of embodiments 1-15, wherein the cell-washing subsystem comprises a second tangential flow filtration system configured to perform the first buffer exchange operation.

17. The system of any one of embodiments 1-16, wherein the cell-washing subsystem comprises a second scale system configured to measure an amount of the second buffer added to the cells during the first buffer exchange operation.

18. The system of any one of embodiments 1-17, wherein the cell-washing subsystem comprises a fourth agitation system configured to agitate and promote homogeneity of one or more of the diluted cell suspension and the cells suspended in the preservation media.

19. The system of any one of embodiments 1-18, wherein the cell-washing subsystem comprises a second illumination system configured to illuminate the one or more of the diluted cell suspension and the cells suspended in the preservation media.

20. The system of any one of embodiments 1-19, wherein the preservation media is a cryo-protectant media.

21. The system of any one of embodiments 1-20, wherein the container-filling subsystem comprises a third scale system configured to measure an amount of the cells suspended in the preservation media added to the one or more containers.

22. The system of any one of embodiments 1-21, wherein the container-filling subsystem comprises a fifth agitation system configured to agitate and promote homogeneity of the cells suspended in the preservation media in the one or more containers.

23. The system of any one of embodiments 1-22, wherein the container-filling subsystem comprises a third illumination system configured to illuminate the cells suspended in the preservation media in the one or more containers.

24. The system of any one of embodiments 1-23, wherein the system is configured such that fluid flows gravitationally between two or more of the subsystems.

25. The system of any one of embodiments 1-34, wherein the system comprises one or more pumps configured to cause fluid to flow between two or more of the subsystems.

26. The system of any one of embodiments 1-25, wherein one or more of the subsystems may be able to be individually to test integrity of one or more components of the subsystem.

27. A method for processing cells, the method performed by a system comprising a suspension preparation subsystem, a cell-deformation subsystem, a dilution subsystem, an incubation subsystem, a cell-washing subsystem, and a container-filling subsystem, the method comprising:
at the suspension preparation subsystem:
receiving cells;
performing a cell isolation operation on the cells;
receiving a delivery media; and
creating a cell suspension by causing the cells to be suspended in the delivery media;
at the cell-deformation subsystem,
receiving flow of the cell suspension from the suspension preparation subsystem; and
causing the cell suspension to flow through one or more cell-deforming constrictions configured to cause perturbations in cell membranes of the cells to allow entry of a payload into the cell;
at the dilution subsystem
receiving flow of the cell suspension from the cell-deformation subsystem;
receiving a fluid or dry reagent; and
creating a diluted cell suspension by causing the cell suspension to be mixed with the fluid or dry reagent;
at the incubation subsystem:
receiving flow of the diluted cell suspension from the dilution subsystem; and
adjusting a temperature of the diluted cell suspension;
   at the cell-washing subsystem:
receiving flow of the diluted cell suspension from the incubation subsystem; and
performing a first buffer exchange operation on the cells to suspend the cells in a preservation media; and
   at the container-filling subsystem:
receiving flow of the cells suspended in preservation media from the cell-washing subsystem; and
causing the cells suspended in preservation media to flow into one or more containers.

28. A disposable kit for use in a system for processing blood, the kit comprising:
a first set of disposable components configured to be usable in a suspension preparation subsystem of the system, wherein the first set of disposable components comprises one or both of:
a spiral inertial separation microfluidic consumable; and
a first tangential flow filtration membrane assembly;
a second set of disposable components configured to be usable in a cell-deformation subsystem of the system, wherein the second set of disposable components comprises one or both of:
a component comprising one or more cell-deforming constrictions through which cells may be forced to cause perturbation of membranes of the cells; and
a cartridge for housing and delivering fluid to and from a component comprising the one or more cell-deforming constructions;
a third set of disposable components configured to be usable in a dilution subsystem of the system, wherein the third set of disposable components comprises a first cell aggregate filter;
a fourth set of disposable components configured to be usable in an incubation subsystem of the system, wherein the fourth set of disposable components comprises a second cell aggregate filter;
a fifth set of disposable components configured to be usable in a cell-washing subsystem of the system, wherein the fifth set of disposable components comprises a second tangential flow filtration membrane assembly; and
a sixth set of disposable components configured to be usable in a container-filling subsystem of the system, wherein the sixth set of disposable components comprises a third cell aggregate filter.

29. The kit of embodiment 28, wherein one or more of the components of one or more of the sets of disposable components of the kit are configured to be fluidly connected to one or more components of the corresponding subsystem of the system.

30. The kit of any one of embodiments 28-29, wherein:
one or more of the disposable components of the second set of disposable components is configured to be fluidly connected to and receive fluid flow from one or more of the disposable components of the first set of disposable components;
one or more of the disposable components of the third set of disposable components is configured to be fluidly connected to and receive fluid flow from one or more of the disposable components of the second set of disposable components;
one or more of the disposable components of the fourth set of disposable components is configured to be fluidly connected to and receive fluid flow from one or more of the disposable components of the third set of disposable components;
one or more of the disposable components of the fifth set of disposable components is configured to be fluidly connected to and receive fluid flow from one or more of the disposable components of the fourth set of disposable components;
one or more of the disposable components of the sixth set of disposable components is configured to be fluidly connected to and receive fluid flow from one or more of the disposable components of the fifth set of disposable components.

31. The kit of any one of embodiments 28-30, wherein the first set of disposable components comprises a cell aggregate filter.

32. The kit of any one of embodiments 28-31, wherein the first set of disposable components comprises a leukoreduction filter.

33. The kit of any one of embodiments 28-32, wherein the first set of disposable components comprises a sampling bulb.

34. The kit of any one of embodiments 28-33, wherein the first set of disposable components comprises one or more of: tubing, a tube fitting, a connector, a clamp, a bag, and a carboy.

35. The kit of any one of embodiments 28-34, wherein the second set of disposable components comprises a rigid sample vessel.

36. The kit of any one of embodiments 28-35, wherein the second set of disposable components comprises a cell-aggregate filter.

37. The kit of any one of embodiments 28-36, wherein the second set of disposable components comprises one or more of: tubing, a tube fitting, a connector, a clamp, and a bag.

38. The kit of any one of embodiments 28-37, wherein the third set of disposable components comprises a sampling bulb.

39. The kit of any one of embodiments 28-38, wherein the third set of disposable components comprises one or more of: tubing, a tube fitting, a connector, a clamp, a bag, and a carboy.

40. The kit of any one of embodiments 28-39, wherein the fourth set of disposable components comprises one or more of: tubing, a tube fitting, a connector, a clamp, and a bag.

41. The kit of any one of embodiments 28-40, wherein the fifth set of disposable components comprises a cell aggregate filter.

42. The kit of any one of embodiments 28-41, wherein the fifth set of disposable components comprises a sampling bulb.

43. The kit of any one of embodiments 28-42, wherein the fifth set of disposable components comprises one or more of: tubing, a tube fitting, a connector, a clamp, a bag, and a carboy.

44. The kit of any one of embodiments 28-43, wherein the sixth set of disposable components comprises a sampling bulb.

45. The kit of any one of embodiments 28-44, wherein the sixth set of disposable components comprises one or more of: tubing, a tube fitting, a connector, a clamp, and a bag.

46. A system for processing cells, the system comprising:
a suspension preparation subsystem configured to receive cells, to perform a cell isolation operation on the cells, to receive a delivery media, and to create a cell suspension by causing the cells to be suspended in the delivery media;
a cell-deformation subsystem in fluid communication with the suspension preparation subsystem, wherein the cell-deformation subsystem is configured to receive the cell suspension from the suspension preparation subsystem and to cause the cell suspension to flow through one or more cell-deforming constrictions configured to cause perturbations in cell membranes of the cells to allow entry of a payload into the cell;
a temperature control subsystem in fluid communication with the cell-deformation subsystem, wherein the temperature control subsystem is configured to receive the cell suspension from the cell-deformation subsystem and to receive a fluid or dry reagent and to create a diluted cell suspension by causing the cell suspension to be mixed with the fluid or dry reagent; and further wherein the temperature control subsystem is configured to adjust a temperature of the diluted cell suspension;
a cell-washing subsystem in fluid communication with the incubation subsystem, wherein the cell-washing subsystem is configured to receive the diluted cell suspension from the incubation subsystem and to perform a first buffer exchange operation on the cells to suspend the cells in a preservation media; and
a container-filling subsystem in fluid communication with the incubation subsystem, wherein the container-filling subsystem is configured to receive the cells suspended in preservation media from the cell-washing subsystem and to cause the cells suspended in preservation media to flow into one or more containers.

### 5. Terminology

As used herein, the terms “about” and “approximately,” when used to modify a numeric value or numeric range, indicate that deviations of up to 10% above and down to 10% below the value or range remain within the intended meaning of the recited value or range. It is understood that wherever aspects are described herein with the language “about” or “approximately” a numeric value or range, otherwise analogous aspects referring to the specific numeric value or range are also provided.

As used herein, the term “disposable” generally refers to a device or component that can be releasably coupled to, for example, a frame, another device, or another component. In some aspects, the point of care systems for processing cells described herein use such disposable components, as described further infra.

As used herein, the terms “communicate” (e.g., a first component “communicates with” or “is in communication with” a second component) and “coupled” (e.g., a first component is “coupled to” a second component) is used herein to indicate a structural, functional, mechanical, optical, electrical, or fluidic relationship, or any combination thereof, between two or more components or elements. As such, the fact that one component is said to communicate with or be coupled to a second component is not intended to exclude the possibility that additional components may be present between and/or operatively associated or engaged with, the first and second components. Moreover, it is noted that the terms “in fluidic communication” and “fluidically connected” are used interchangeably herein.

The invention is not to be limited in scope by the specific aspects described herein. Indeed, various modifications of the invention in addition to those described will become apparent to those skilled in the art from the foregoing description and accompanying figures. Such modifications are intended to fall within the scope of the appended claims.

1. A system for processing cells, the system comprising:
a frame comprising a plurality of spatially distinct zones and a plurality of subsystems;
a first zone of the frame for receiving a suspension preparation subsystem, the suspension preparation system comprising:
a) a first cell suspension inlet upstream of and fluidically coupled to a cell suspension device, the cell suspension device configured to suspend cells in a liquid medium thereby creating a cell suspension, and
b) a first cell suspension outlet downstream of and fluidically coupled to the cell suspension device;
a second zone of the frame for receiving a cell-deformation subsystem directly downstream of and couplable with the suspension preparation subsystem, the cell-deformation subsystem comprising:
a) a second cell suspension inlet fluidically couplable with the first cell suspension outlet,
b) one or more cell-deforming constrictions fluidically coupled with and downstream of the second cell suspension inlet configured to cause perturbations in cell membranes of the cells to allow entry of a payload into the cell, and
c) a second cell suspension outlet downstream of and fluidically coupled to the one or more cell-deforming constrictions;
a third zone of the frame for receiving a dilution subsystem directly downstream of and couplable with the cell-deformation subsystem, the dilution subsystem comprising:
a) a third cell suspension inlet fluidically couplable with the second cell suspension outlet,
b) a buffer inlet,
c) a first container fluidically coupled with the third cell suspension inlet and the buffer inlet and configured to receive a cell suspension from the cell-deformation subsystem and to receive a fluid or a dry reagent that mixes with the cell suspension to create a diluted cell suspension, and
d) a diluted cell suspension outlet downstream of and fluidically coupled to the first container;
a fourth zone of the frame for receiving an incubation subsystem directly downstream of and couplable with the dilution subsystem, the incubation subsystem comprising:
a) a diluted cell suspension inlet fluidically couplable with the diluted cell suspension outlet;
b) a second container fluidically coupled with the diluted cell suspension inlet and configured to receive the diluted cell suspension,
c) a plate configured to adjust the temperature of the diluted cell suspension in the container to create an incubated cell suspension, and
d) an incubated cell suspension outlet downstream of and fluidically coupled to the second container;
a fifth zone of the frame for receiving a cell-washing subsystem directly downstream of and couplable with the incubation subsystem, the cell-washing subsystem comprising:
a) an incubated cell suspension inlet fluidically couplable with the incubated cell suspension outlet,
b) a preservation media inlet,
c) a third container fluidically coupled with the incubated cell suspension inlet and the preservation media inlet and configured to receive the incubated cell suspension and to receive preservation media that mixes with the incubated cell suspension in the container thereby suspending the cells in preservation media, and
d) a washed cell suspension outlet downstream of and fluidically coupled to the third container;
a sixth zone of the frame for receiving a container-filling subsystem directly downstream of and couplable with the incubation subsystem, the container-filling subsystem comprising:
a) an inlet fluidically couplable with the washed cell suspension outlet, and
b) one or more containers fluidically couplable with the inlet and configured to receive cells suspended in preservation media; and
one or more pumps configured to move the cell suspension between the fluidically connected subsystems along a flow path and through the system such that a residency time of the cell suspension in each of the subsystems is less than a day,
wherein the plurality of subsystems comprise at least the cell suspension preparation system, the cell-deformation subsystem, the incubation subsystem, and the container filing subsystem,
wherein the dilution subsystem is omittable from the system, and if omitted the second cell suspension outlet of the cell deformation subsystem is directly couplable with the diluted cell suspension inlet of the incubation subsystem,
wherein the cell-washing subsystem is omittable from the system, and if omitted the incubated cell suspension outlet of the incubation subsystem is directly couplable with the suspension preparation subsystem to exchange the incubated cell suspension into the preservation media.

2. The system for processing cells of claim 1, wherein the one or more pumps are configured to move the cell suspension:
a) along the flow path and through the system in less than a day; or
b) along the flow path and through the system in about 5 hours to about 7 hours.

3. The system for processing cells of claim 1, wherein the flow path is sterile and the system is configured to be used in a non-sterile location.

4. The system for processing cells of claim 1, wherein the suspension preparation subsystem further comprises:
a) a delivery media inlet fluidically coupled to and upstream of the cell suspension device;
and/or b) a dilution media inlet; and/or
c) a wash media inlet.

5. The system for processing cells of claim 1, wherein the inlets and outlets of each of the plurality of subsystems are respectively associated with tubing, and the tubing of an outlet of an upstream subsystem is fluidically couplable with the tubing of an inlet of a downstream subsystem.

6. The system for processing cells of claim 1, further comprising a weld-offable sample bulb associated with at least one of the plurality of subsystems for analyzing the cells of the at least one subsystem.

7. The system for processing cells of claim 1, wherein one or more of the plurality of subsystems are controllable independently of one or more of the other subsystems.

8. The system for processing cells of claim 1, wherein the suspension preparation subsystem further comprises a tangential flow filter assembly and/or an agitation system.

9. The system for processing cells of claim 1, wherein one or more of the plurality of subsystems are releasably supported by the frame.

10. The system for processing cells of claim 1, wherein a first of the plurality of subsystems is releasably couplable with a second of the plurality of subsystems downstream thereof.

11. A point of care system for processing cells, the system comprising:
a frame;
a suspension preparation subsystem releasably coupled to the frame by a fastening element, the suspension preparation subsystem comprising at least one outlet and at least one container, wherein the at least one outlet is configured to be coupled to the at least one container for receiving cells suspended in delivery media;
a cell-deformation subsystem releasably coupled to the frame by a fastening element and in fluid communication with the suspension preparation subsystem;
a temperature control subsystem releasably coupled to the frame by a fastening element and in fluid communication with the cell-deformation subsystem;
a cell-washing subsystem releasably coupled to the frame by a fastening element and in direct fluid communication with the temperature control subsystem, the cell washing subsystem comprising a device configured to exchange a cell suspension buffer for a cell preservation medium;
a container-filling subsystem releasably coupled to the frame by a fastening element and in fluid communication with the cell-washing subsystem; and
one or more pumps configured to move a solution comprising processed cells between the fluidically connected subsystems along a flow path and through the system in less than a day,
wherein the subsystems are localized to a plurality of zones on the frame, and
wherein a first zone of the plurality of zones contains first tubing associated to the respective subsystem, a second zone of the plurality of zones contains second tubing associated to the respective subsystem, and the respective subsystems of the first zone and second zone are directly fluidically couplable by the first and second tubing.

12. The point of care system of claim 11, wherein the container is a bag comprising at least one inlet and at least one outlet.

13. The system of claim 11, wherein the cell deformation subsystem comprises a pressurization system configured to generate pressure to force a cell suspension through one or more cell-deforming constrictions.

14. The point of care system of claim 11, wherein each of the fastening elements is one of a hook, a hook-and-loop fastener, a temporary adhesive, a tie, or a pin, and at least one of the fastening elements is connecting to a scale system for measuring the weight of at least one container in the point of care system.

15. The point of care system of claim 11, wherein each of the plurality of zones comprises a scale system or a temperature-controlled plate.

16. The point of care system of claim 11, further comprising a waste container, wherein waste from two or more of the plurality of zones flows into the waste container.

17. The system for processing cells of claim 1, wherein the cells comprise:
(a) red blood cells (RBC);
(b) red blood cell-derived vesicles;
(c) peripheral blood mononuclear cells (PBMCs);
(d) T cells, B cells, dendritic cells, monocytes, macrophages, eosinophils, basophils, natural killer (NK) cells, natural killer T (NKT) cells, mast cells or neutrophils; or
(e) antigen presenting cells (APCs).

18. A kit for use in a system for processing cells, the kit comprising one or more of
i. a first kit comprising releasably couplable components configured to be releasably couplable to a frame of a suspension preparation subsystem of the system, wherein the first set of releasably couplable components comprises a cell isolation device and/or a cell suspension device, the cell isolation device and the cell suspension device comprising an inlet and an outlet respectively associated to tubing;
ii. a second kit comprising releasably couplable components configured to be releasably couplable to a frame of a cell-deformation subsystem of the system, wherein the second set of releasably couplable components comprises one or more microfluidic chips comprising one or more cell-deforming constrictions through which cells may be forced to cause perturbation of membranes of the cells, the one or more microfluidic chips comprising an inlet and an outlet respectively associated to tubing;
iii. a third kit comprising releasably couplable components configured to be releasably couplable to a frame of a dilution subsystem of the system, wherein the third set of releasably couplable components comprises a container and at least one cell aggregate filter, the container and the at least one cell aggregate filter comprising an inlet and an outlet respectively associated to tubing;
iv. a fourth kit comprising releasably couplable components configured to be releasably couplable to a frame of an incubation subsystem of the system, wherein the fourth set of releasably couplable components comprises a container and at least one cell aggregate filter, the container and the at least one cell aggregate filter comprising an inlet and an outlet respectively associated to tubing;
v. a fifth kit comprising releasably couplable components configured to be releasably couplable to a frame of a cell-washing subsystem of the system, wherein the fifth set of releasably couplable components comprises a container and a tangential flow filtration membrane assembly, the container and the tangential flow filtration membrane assembly comprising an inlet and an outlet respectively associated to tubing; and
vi. a sixth kit comprising releasably couplable components configured to be releasably couplable to a frame of a container-filling subsystem of the system, wherein the sixth set of releasably couplable components comprises at least one cell aggregate filter, the at least one cell aggregate filter comprising an inlet and an outlet respectively associated to tubing.

19. The kit of claim 18, wherein the first kit of releasably couplable components comprises one or more of: additional tubing, a tube fitting, a connector, a clamp, a sampling bulb, a carboy, an air filter, and a tangential flow filtration filter assembly.

20. The kit of claim 18, wherein the second kit of releasably couplable components comprises one or more microfluidic chip cartridges for holding the one or more microfluidic chips.
